(19) United States
(12) Patent Application Publication
(10) Pub. No.: US 2023/0075277 A1
(43) Pub. Date: Mar. 9, 2023

(54) AIR PREHEATER FOR HUMIDIFICATION TUB

(71) Applicant: ResMed Pty Ltd, Bella Vista (AU)

(72) Inventor: Simon Robert CORK, Sydney (AU)

(21) Appl. No.: 17/822,315

(22) Filed: Aug. 25, 2022

Related U.S. Application Data
(60) Provisional application No. 63/237,241, filed on Aug. 26, 2021.

(51) Int. Cl. A61M 16/10; A61M 16/16

(52) U.S. Cl. CPC A61M 16/109; A61M 16/16; A61M 2205/121; A61M 2205/123

(57) ABSTRACT

An apparatus for providing a humidified flow of pressurized breathable gas includes a humidification chamber having a heatable base surface and a humidification tub configured to contain a supply of water. The humidification tub includes a thermally conductive base and is removably positionable at least partially within the humidification chamber and onto the heatable base surface in order to allow for heating of the water in the humidification tub. The humidification chamber is configured to receive the flow of pressurized breathable gas and output the flow of pressurized breathable gas with increased humidity. The flow of pressurized breathable gas is pre-heated before entering the humidification tub by being configured to, after entering the humidification chamber and before entering the humidification tub, flow along at least a portion of the perimeter of the humidification tub.

### 1 CROSS-REFERENCE TO RELATED APPLICATIONS

This application claims the benefit of U.S. Provisional Application No. 63/237,241, filed Aug. 26, 2021, the entire contents of which is incorporated by reference herein in its entirety.

### 2 BACKGROUND OF THE TECHNOLOGY

### 2.1 Field of the Technology

The present technology relates to one or more of the screening, diagnosis, monitoring, treatment, prevention and amelioration of respiratory-related disorders. The present technology also relates to medical devices or apparatus, and their use.

### 2.2 Description of the Related Art

### 2.2.1 Human Respiratory System and its Disorders

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the inhaled air into the venous blood and carbon dioxide to move in the opposite direction. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See “Respiratory Physiology”, by John B. West, Lippincott Williams & Wilkins, 9th edition published 2012.

A range of respiratory disorders exist. Certain disorders may be characterised by particular events, e.g. apneas, hypopneas, and hyperpneas.

Examples of respiratory disorders include Obstructive Sleep Apnea (OSA), Cheyne-Stokes Respiration (CSR), respiratory insufficiency, Obesity Hyperventilation Syndrome (OHS), Chronic Obstructive Pulmonary Disease (COPD), Neuromuscular Disease (NMD) and Chest wall disorders.

Obstructive Sleep Apnea (OSA), a form of Sleep Disordered Breathing (SDB), is characterised by events including occlusion or obstruction of the upper air passage during sleep. It results from a combination of an abnormally small upper airway and the normal loss of muscle tone in the region of the tongue, soft palate and posterior oropharyngeal wall during sleep. The condition causes the affected patient to stop breathing for periods typically of 30 to 120 seconds in duration, sometimes 200 to 300 times per night. It often causes excessive daytime somnolence, and it may cause cardiovascular disease and brain damage. The syndrome is a common disorder, particularly in middle aged overweight males, although a person affected may have no awareness of the problem. See U.S. Pat. No. 4,944,310 (Sullivan).

Respiratory failure is an umbrella term for respiratory disorders in which the lungs are unable to inspire sufficient oxygen or exhale sufficient CO2 to meet the patient's needs. Respiratory failure may encompass some or all of the following disorders.

A patient with respiratory insufficiency (a form of respiratory failure) may experience abnormal shortness of breath on exercise.

A range of therapies have been used to treat or ameliorate such conditions. Furthermore, otherwise healthy individuals may take advantage of such therapies to prevent respiratory disorders from arising. However, these have a number of shortcomings.

### 2.2.2 Therapies

Various respiratory therapies, such as Continuous Positive Airway Pressure (CPAP) therapy, Non-invasive ventilation (NIV), Invasive ventilation (IV), and High Flow Therapy (HFT) have been used to treat one or more of the above respiratory disorders.

### 2.2.2.1 Respiratory Pressure Therapies

Respiratory pressure therapy is the application of a supply of air to an entrance to the airways at a controlled target pressure that is nominally positive with respect to atmosphere throughout the patient's breathing cycle (in contrast to negative pressure therapies such as the tank ventilator or cuirass).

Continuous Positive Airway Pressure (CPAP) therapy has been used to treat Obstructive Sleep Apnea (OSA). The mechanism of action is that continuous positive airway pressure acts as a pneumatic splint and may prevent upper airway occlusion, such as by pushing the soft palate and tongue forward and away from the posterior oropharyngeal wall. Treatment of OSA by CPAP therapy may be voluntary, and hence patients may elect not to comply with therapy if they find devices used to provide such therapy one or more of: uncomfortable, difficult to use, expensive and aesthetically unappealing.

### 2.2.2.2 Flow Therapies

Not all respiratory therapies aim to deliver a prescribed therapeutic pressure. Some respiratory therapies aim to deliver a prescribed respiratory volume, by delivering an inspiratory flow rate profile over a targeted duration, possibly superimposed on a positive baseline pressure. In other cases, the interface to the patient's airways is ‘open’ (unsealed) and the respiratory therapy may only supplement the patient's own spontaneous breathing with a flow of conditioned or enriched gas. In one example, High Flow therapy (HFT) is the provision of a continuous, heated, humidified flow of air to an entrance to the airway through an unsealed or open patient interface at a “treatment flow rate” that may be held approximately constant throughout the respiratory cycle. The treatment flow rate is nominally set to exceed the patient's peak inspiratory flow rate. HFT has been used to treat OSA, CSR, respiratory failure, COPD, and other respiratory disorders. One mechanism of action is that the high flow rate of air at the airway entrance improves ventilation efficiency by flushing, or washing out, expired CO2 from the patient's anatomical deadspace. Hence, HFT is thus sometimes referred to as a deadspace therapy (DST). Other benefits may include the elevated warmth and humidification (possibly of benefit in secretion management) and the potential for modest elevation of airway pressures. As an alternative to constant flow rate, the treatment flow rate may follow a profile that varies over the respiratory cycle.

Another form of flow therapy is long-term oxygen therapy (LTOT) or supplemental oxygen therapy. Doctors may prescribe a continuous flow of oxygen enriched air at a specified oxygen concentration (from 21%, the oxygen fraction in ambient air, to 100%) at a specified flow rate (e.g., 1 litre per minute (LPM), 2 LPM, 3 LPM, etc.) to be delivered to the patient's airway.

### 2.2.2.3 Supplementary Oxygen

For certain patients, oxygen therapy may be combined with a respiratory pressure therapy or HFT by adding supplementary oxygen to the pressurised flow of air. When oxygen is added to respiratory pressure therapy, this is referred to as RPT with supplementary oxygen. When oxygen is added to HFT, the resulting therapy is referred to as HFT with supplementary oxygen.

### 2.2.3 Respiratory Therapy Systems

These respiratory therapies may be provided by a respiratory therapy system or device. Such systems and devices may also be used to screen, diagnose, or monitor a condition without treating it.

A respiratory therapy system may comprise a Respiratory Pressure Therapy Device (RPT device), an air circuit, a humidifier, a patient interface, an oxygen source, and data management.

### 2.2.3.1 Patient Interface

A patient interface may be used to interface respiratory equipment to its wearer, for example by providing a flow of air to an entrance to the airways. The flow of air may be provided via a mask to the nose and/or mouth, a tube to the mouth or a tracheostomy tube to the trachea of a patient. Depending upon the therapy to be applied, the patient interface may form a seal, e.g., with a region of the patient's face, to facilitate the delivery of gas at a pressure at sufficient variance with ambient pressure to effect therapy, e.g., at a positive pressure of about 10 cmH₂O relative to ambient pressure. For other forms of therapy, such as the delivery of oxygen, the patient interface may not include a seal sufficient to facilitate delivery to the airways of a supply of gas at a positive pressure of about 10 cmH₂O. For flow therapies such as nasal HFT, the patient interface is configured to insufflate the nares but specifically to avoid a complete seal. One example of such a patient interface is a nasal cannula.

### 2.2.3.2 Respiratory Pressure Therapy (RPT) Device

A respiratory pressure therapy (RPT) device may be used individually or as part of a system to deliver one or more of a number of therapies described above, such as by operating the device to generate a flow of air for delivery to an interface to the airways. The flow of air may be pressure-controlled (for respiratory pressure therapies) or flow-controlled (for flow therapies such as HFT). Thus RPT devices may also act as flow therapy devices. Examples of RPT devices include a CPAP device and a ventilator.

The designer of a device may be presented with an infinite number of choices to make. Design criteria often conflict, meaning that certain design choices are far from routine or inevitable. Furthermore, the comfort and efficacy of certain aspects may be highly sensitive to small, subtle changes in one or more parameters.

### 2.2.3.3 Air Circuit

An air circuit is a conduit or a tube constructed and arranged to allow, in use, a flow of air to travel between two components of a respiratory therapy system such as the RPT device and the patient interface. In some cases, there may be separate limbs of the air circuit for inhalation and exhalation. In other cases, a single limb air circuit is used for both inhalation and exhalation.

### 2.2.3.4 Humidifier

Delivery of a flow of air without humidification may cause drying of airways. The use of a humidifier with an RPT device and the patient interface produces humidified gas that minimizes drying of the nasal mucosa and increases patient airway comfort. In addition, in cooler climates, warm air applied generally to the face area in and about the patient interface is more comfortable than cold air.

A range of artificial humidification devices and systems are known, however they may not fulfil the specialised requirements of a medical humidifier.

Medical humidifiers are used to increase humidity and/or temperature of the flow of air in relation to ambient air when required, typically where the patient may be asleep or resting (e.g. at a hospital). A medical humidifier for bedside placement may be small. A medical humidifier may be configured to only humidify and/or heat the flow of air delivered to the patient without humidifying and/or heating the patient's surroundings. Room-based systems (e.g. a sauna, an air conditioner, or an evaporative cooler), for example, may also humidify air that is breathed in by the patient, however those systems would also humidify and/or heat the entire room, which may cause discomfort to the occupants. Furthermore, medical humidifiers may have more stringent safety constraints than industrial humidifiers

While a number of medical humidifiers are known, they can suffer from one or more shortcomings. Some medical humidifiers may provide inadequate humidification, some are difficult or inconvenient to use by patients.

### 2.2.3.5 Data Management

There may be clinical reasons to obtain data to determine whether the patient prescribed with respiratory therapy has been “compliant”, e.g. that the patient has used their RPT device according to one or more “compliance rules”. One example of a compliance rule for CPAP therapy is that a patient, in order to be deemed compliant, is required to use the RPT device for at least four hours a night for at least 21 of 30 consecutive days. In order to determine a patient's compliance, a provider of the RPT device, such as a health care provider, may manually obtain data describing the patient's therapy using the RPT device, calculate the usage over a predetermined time period, and compare with the compliance rule. Once the health care provider has determined that the patient has used their RPT device according to the compliance rule, the health care provider may notify a third party that the patient is compliant.

There may be other aspects of a patient's therapy that would benefit from communication of therapy data to a third party or external system.

Existing processes to communicate and manage such data can be one or more of costly, time-consuming, and error-prone.

### 2.2.3.6 Vent Technologies

Some forms of treatment systems may include a vent to allow the washout of exhaled carbon dioxide. The vent may allow a flow of gas from an interior space of a patient interface, e.g., the plenum chamber, to an exterior of the patient interface, e.g., to ambient.

### 2.2.4 Screening, Diagnosis, and Monitoring Systems

Polysomnography (PSG) is a conventional system for diagnosis and monitoring of cardio-pulmonary disorders, and typically involves expert clinical staff to apply the system. PSG typically involves the placement of 15 to 20 contact sensors on a patient in order to record various bodily signals such as electroencephalography (EEG), electrocardiography (ECG), electrooculograpy (EOG), electromyography (EMG), etc. PSG for sleep disordered breathing has involved two nights of observation of a patient in a clinic, one night of pure diagnosis and a second night of titration of treatment parameters by a clinician. PSG is therefore expensive and inconvenient. In particular, it is unsuitable for home screening/diagnosis/monitoring of sleep disordered breathing.

Screening and diagnosis generally describe the identification of a condition from its signs and symptoms. Screening typically gives a true/false result indicating whether or not a patient's SDB is severe enough to warrant further investigation, while diagnosis may result in clinically actionable information. Screening and diagnosis tend to be one-off processes, whereas monitoring the progress of a condition can continue indefinitely. Some screening/diagnosis systems are suitable only for screening/diagnosis, whereas some may also be used for monitoring.

Clinical experts may be able to screen, diagnose, or monitor patients adequately based on visual observation of PSG signals. However, there are circumstances where a clinical expert may not be available, or a clinical expert may not be affordable. Different clinical experts may disagree on a patient's condition. In addition, a given clinical expert may apply a different standard at different times.

### 3 BRIEF SUMMARY OF THE TECHNOLOGY

The present technology is directed towards providing medical devices used in the screening, diagnosis, monitoring, amelioration, treatment, or prevention of respiratory disorders having one or more of improved comfort, cost, efficacy, ease of use and manufacturability.

A first aspect of the present technology relates to apparatus used in the screening, diagnosis, monitoring, amelioration, treatment or prevention of a respiratory disorder.

Another aspect of the present technology relates to methods used in the screening, diagnosis, monitoring, amelioration, treatment or prevention of a respiratory disorder.

An aspect of certain forms of the present technology is to provide methods and/or apparatus that improve the compliance of patients with respiratory therapy.

One form of the present technology comprises an airflow pathway inside of a humidifier through which pressurized breathable gas (e.g., exiting from a flow generator) is pre-heated and configured to flow prior to entering a tub.

In examples, the airflow pathway is between a wall of the chamber and a wall of the tub, which is received within the chamber.

In examples, the entrance to the airflow pathway is disposed at least approximately 180°, and up to approximately 360° away from the exit to the airflow pathway around the perimeter of the tub.

In examples, a heater plate is disposed proximate to the airflow pathway and is configured to pre-heat, e.g., convectively heat, pressurized breathable gas flowing through the airflow pathway.

Another aspect of one form of the present technology is an airflow pathway formed between a chamber side wall and a tub side wall, which is configured to receive a flow of pressurized gas prior to being introduced into a tub inlet.

In examples, a heating element forms an inferior portion of the airflow pathway and is configured to heat, e.g., convectively heat, pressurized breathable gas flowing through the airflow pathway.

Another aspect of one form of the present technology is a sealed airflow pathway within a humidifier chamber that extends between a chamber inlet and a tub inlet.

In examples, pressurized breathable gas is configured to increase in temperature while flowing through the airflow pathway.

In examples, seals form upper and/or lower boundaries in of the airflow pathway, and are configured to limit the pressurized breathable gas from flowing outside of the airflow pathway.

In examples, the seals are configured to sealingly engage a tub and/or a chamber when the tub is removably positioned within the chamber.

Another aspect of one form of the present technology is configured to pre-heat, e.g., convectively heat, pressurized breathable gas prior to being introduced into a tub inlet.

Another aspect of one form of the present technology is an airflow pathway for pre-heating a flow of pressurized breathable gas, which is positioned upstream from a humidifier tub and configured to be positioned downstream from a flow generator.

Another aspect of one form of the present technology is configured to passively heat a flow of pressurized breathable gas prior to the flow entering a humidifier tub.

In examples, the flow of pressurized breathable gas is heated by excess heat generated from a heating element used to directly heat the humidifier tub.

Another form of the present technology is a humidifier for humidifying a flow of pressurized breathable gas to be delivered to a patient, the humidifier comprising:

a chamber comprising:
a chamber base having a chamber bottom surface and at least one vertical chamber side wall extending upwardly from the chamber bottom surface, the at least one vertical side wall having a chamber opening, and
a chamber lid being hingedly attached to the chamber base and pivotably movable between an open position and a closed position,

a tub configured to contain a supply of water, the tub being removably positionable within the chamber base, the tub configured to receive the flow of pressurized breathable gas and output the flow of pressurized breathable gas with increased humidity, the tub comprising:
a tub base configured to contain the supply of water, the tub base having a tub bottom surface and at least one vertical tub side wall extending upwardly from the tub bottom surface, the tub bottom surface configured to contact the chamber bottom surface, and
a tub opening configured to communicate with the chamber opening;

a heater plate positioned within the chamber and configured to contact the tub base;

a first pathway seal contacting the at least one vertical chamber side wall and the at least one tub vertical side wall in a sealing arrangement in use; and

a pathway formed between the at least one vertical chamber side wall and the at least one vertical tub side wall, the pathway extending at least partly around a perimeter of the tub, the pathway comprising:
a pathway entrance in direct communication with the chamber opening, and
a pathway exit in direct communication with the tub opening;

wherein:

the chamber opening is configured to direct the flow of pressurized breathable gas through the pathway entrance, and the first pathway seal is configured to direct the flow of pressurized breathable gas around the pathway toward the pathway exit; and

the heater plate is configured to generate heat in order to heat, e.g., convectively heat, the pressurized breathable gas so that a first air temperature within the pathway proximate to the pathway entrance is less than a second air temperature within the pathway proximate to the pathway exit.

Another form of the present technology is a humidifier for humidifying a flow of pressurized breathable gas to be delivered to a patient, the humidifier comprising:

a chamber comprising:
a chamber base having a chamber bottom surface and at least one vertical chamber side wall extending upwardly from the chamber bottom surface, the at least one vertical side wall having a chamber opening configured to receive the flow of pressurized breathable gas from a flow generator, and
a chamber lid being hingedly attached to the chamber base and pivotably movable between an open position and a closed position,

a tub configured to contain a supply of water, the tub being removably positionable within the chamber base, the tub configured to receive the flow of pressurized breathable gas and output the flow of pressurized breathable gas with increased humidity, the tub comprising:
a tub base configured to contain the supply of water, the tub base having a tub bottom surface and at least one vertical tub side wall extending upwardly from the tub bottom surface, the tub bottom surface configured to contact the chamber bottom surface, and
a tub opening configured to provide communication to an interior of the tub base;

a heater plate positioned within the chamber and configured to contact the tub base; and

a pathway formed between the at least one vertical chamber side wall and the at least one vertical tub side wall, the pathway extending at least partly around a perimeter of the tub, the pathway comprising:

wherein:

the flow of pressurized breathable gas is configured to enter the chamber through the chamber opening, flow through the pathway, and enter the tub opening after exiting the pathway; and

the temperature of the flow of pressurized breathable gas entering the tub opening is configured to be warmer than the temperature of the flow of pressurized breathable gas entering the chamber opening.

An apparatus for providing humidified flow of pressurized breathable gas to be delivered to a patient, the apparatus comprising:

a humidification chamber having a heatable base surface,

a humidification tub configured to contain a supply of water and a thermally conductive base, the tub being removably positionable at least partially within the humidification chamber and onto the heatable base surface, so as to allow heating of the water in the tub, the tub being configured to receive the flow of pressurized breathable gas and output the flow of pressurized breathable gas with increased humidity;

wherein the flow of pressurized breathable gas is pre-heated before entering the humidification tub by being configured to, after entering the humidification chamber and before entering the humidification tub, flow along at least a portion of the perimeter of the humidification tub.

An apparatus for providing humidified flow of pressurized breathable gas to be delivered to a patient, the apparatus comprising:

a humidification chamber having a heatable base surface forming an outermost surface and at least one chamber side wall extending from the heatable base surface;

a humidification tub configured to contain a supply of water, the humidification tub including a thermally conductive base and at least one tub side wall extending from the thermally conductive base, the humidification tub being removably positionable at least partially within the humidification chamber where the thermally conductive base contacts the heatable base surface, so as to allow heating of the water in the tub, the tub being configured to receive the flow of pressurized breathable gas and output the flow of pressurized breathable gas with increased humidity;

wherein the flow of pressurized breathable gas is pre-heated in the humidification chamber before entering the humidification tub by being configured to, after entering the humidification chamber and before entering the humidification tub, flow along at least a portion of the perimeter of the humidification tub.

An apparatus for providing a humidified flow of pressurized breathable gas to be delivered to a patient, the apparatus comprising:

a humidification chamber having a heatable base surface and at least one chamber side wall extending from the heatable base surface;

a humidification tub configured to contain a supply of water, the humidification tub including a thermally conductive base and at least one tub side wall extending from the thermally conductive base, the humidification tub being removably positionable at least partially within the humidification chamber where the thermally conductive base contacts the heatable base surface, so as to allow heating of the water in the humidification tub, the humidification tub being configured to receive the humidified flow of pressurized breathable gas and output the flow of pressurized breathable gas with increased humidity;

wherein the arrangement is such that the flow of pressurized breathable gas is configured to, after entering the humidification chamber and before entering the humidification tub, follow a pathway along at least a portion of a perimeter of the humidification tub, the pathway being located inside the humidification chamber but outside the humidification tub, the propagation of the pressurized breathable gas along the pathway causing the pressurized breathable gas to be pre-heated in the humidification chamber before entering the humidification tub.

In one example, a pathway is formed between the at least one chamber base or side wall and the at least one tub base or side wall, and the flow of pressurized breathable gas is configured to flow along at least a portion of a perimeter of the humidification tub through the pathway.

In examples:

a) The pathway extends at least 180° around the perimeter of the tub;

b) The pathway extends approximately 360° around the perimeter of the tub;

c) The first pathway seal is a face seal;

d) The pathway is at least partially defined by a first pathway seal, the first pathway seal contacts at least portions of the at least one tub side wall when the humidification tub is positioned within the humidification chamber;

e) The first pathway seal forms a superior boundary of the pathway in use;

f) A second pathway seal spaced apart from the first pathway seal and contacting the at least one chamber side wall and the at least one tub side wall in a sealing arrangement, in use;

g) The second pathway seal forms an inferior boundary of the pathway in use;

h) The second pathway seal is a face seal;

i) The second pathway seal is integrally formed on the at least one chamber side wall, and is configured to contact a chamfered edge of the at least one tub side wall when the humidification tub is positioned within the humidification chamber;

j) The second pathway seal contacts the heatable base surface (e.g., a heater plate of the heatable base surface), in use;

k) A bypass seal disposed proximate to the pathway entrance, and configured to form a flow path in a single direction along the pathway;

l) A chamber wall seal surrounding the chamber opening;

m) The chamber lid further includes a chamber lid seal;

n) The chamber lid seal is configured to pressurized a volume of the tub base with the flow of the pressurized breathable gas;

o) The chamber lid seal is configured to seal radially outside of the pathway;

p) A first width between the at least one chamber side wall and the at least one tub side wall at the pathway entrance is less than a second width between the at least one chamber side wall and the at least one tub side wall at the pathway exit;

q) The humidification chamber further includes a chamber lid hingedly attached to the at least one chamber side wall and pivotably movable between an open position and a closed position;

r) The chamber lid includes an entry opening configured to cooperate with the pathway exit when the chamber lid is in the closed position;

s) The entry opening includes a substantially vertical portion and a substantially horizontal portion, the substantially vertical portion configured to extend into the pathway exit, and the substantially horizontal portion extending at least partially over a tub opening configured to provide communication to an interior of the humidification tub;

t) The entry opening is more superior than the pathway, in use;

u) The first pathway seal is more inferior than a chamber opening, in use, the chamber opening configured to receive the flow of pressurized breathable gas from a flow generator;

v) A lid closure assembly to selectively lock the chamber lid to the chamber base;

w) The lid closure assembly includes a lid opening member, a spring, and a latch;

x) The lid opening member is slidably coupled to the at least one chamber side wall (or generally the chamber base) to move the latch between a locked position and an unlocked position, the latch being configured to mechanically engage a catch of the chamber lid in the locked position in order to retain the chamber lid in the closed position, the spring biasing the latch into the locked position;

y) The thermally conductive base includes a metal surface;

z) A first pathway seal contacts the at least one chamber side wall and the at least one tub side wall in a sealing arrangement in use;

aa) The first pathway seal is configured to direct the flow of pressurized breathable gas around the pathway toward an inlet of the humidification tub;

ab) The heater plate is configured to generate heat in order to directly heat the tub base, and wherein the heater plate is configured to passively heat the flow of pressurized breathable gas with the heat generated to directly heat the tub base;

ac) The flow of pressurized breathable gas is directed to flow through the pathway along at least a portion of the perimeter of at least a lower portion of the humidification tub;

ad) Each of the at least one chamber side wall and the at least one tub side wall is a side wall or a face wall;

ae) The at least one chamber side wall is perpendicular to the heatable base surface and the at least one chamber side wall is configured to be vertically oriented in use; and/or

af) The at least one tub side wall is perpendicular to the thermally conductive base and the at least one tub side wall is configured to be vertically oriented in use.

Another form of the present technology is an apparatus of any of the previous forms further comprising:

a flow generator having a flow generator outlet, and being configured to supply the flow of pressurized breathable gas;

wherein:

the flow generator outlet is configured to couple to the humidification chamber (e.g., a chamber opening) in order form a fluid pathway between the flow generator and the humidification chamber.

In examples:

a) The flow generator is integrally coupled to the humidifier;

b) The flow generator is detachably coupled to the humidifier;

c) The flow generator outlet contacts the chamber wall seal in a sealing arrangement; and/or

d) The flow generator outlet is more superior than the first pathway seal, in use.

Another form of the present technology is a humidifier for humidifying a flow of pressurized breathable gas to be delivered to a patient, the humidifier comprising:

a chamber having a chamber opening configured to receive the flow of pressurized breathable gas from a flow generator;

a tub configured to contain a supply of water, the tub configured to receive the flow of pressurized breathable gas and output the flow of pressurized breathable gas with increased humidity, the tub including a tub opening;

a heater plate positioned within the chamber and configured to contact the tub;

wherein:

the flow of pressurized breathable gas is configured to enter the chamber through the chamber opening, and enter the tub through the tub opening, the chamber opening being positioned downstream from the tub opening; and

the temperature of the flow of pressurized breathable gas entering the tub opening is warmer than the temperature of the flow of pressurized breathable gas entering the chamber opening.

Another form of the present technology is a humidifier for humidifying a flow of pressurized breathable gas to be delivered to a patient, the humidifier comprising:

a chamber;

a tub configured to contain a supply of water, the tub configured to receive the flow of pressurized breathable gas and output the flow of pressurized breathable gas with increased humidity; and

a heater plate positioned within the chamber and configured generate heat in order to directly heat the tub while in contact with the tub;

wherein the heater plate is configured to indirectly heat the flow of pressurized breathable gas with the heat generated to directly heat the tub.

Another form of the present technology is a humidifier for humidifying a flow of pressurized breathable gas to be delivered to a patient, the humidifier comprising:

a chamber comprising:
a chamber base, and
a chamber lid being coupled to the chamber base;

a tub configured to contain a supply of water, the tub being removably positionable within the chamber base, the tub configured to receive the flow of pressurized breathable gas and output the flow of pressurized breathable gas with increased humidity; and

a heater plate positioned within the chamber and configured to contact the tub base and heat the supply of water.

Another form of the present technology is a humidifier for humidifying a flow of pressurized breathable gas to be delivered to a patient, the humidifier comprising:

a chamber

a tub configured to contain a supply of water, the tub being removably positionable within the chamber, the tub configured to receive the flow of pressurized breathable gas and output the flow of pressurized breathable gas with increased humidity;

a heater plate positioned within the chamber and configured to contact the tub;

an airflow pathway formed between the chamber and the tub, the airflow pathway extending at least partly around a perimeter of the tub.

Another form of the present technology is an apparatus for delivering the flow of pressurized breathable gas to the patient, the apparatus comprising:

a flow generator having a flow generator outlet, and being configured to supply the flow of pressurized breathable gas; and

the humidifier for humidifying a flow of pressurized breathable gas to be delivered to the patient, the humidifier including a tub;

wherein:

the flow generator outlet is disposed upstream from the humidifier, and the flow of pressurized breathable gas is configured to flow from the flow generator outlet to the humidifier; and

the temperature of the flow of pressurized breathable gas entering the tub is warmer than the temperature of the flow of pressurized breathable gas exiting the flow generator outlet.

Another aspect of one form of the present technology is a patient interface that is moulded or otherwise constructed with a perimeter shape which is complementary to that of an intended wearer.

An aspect of one form of the present technology is a method of manufacturing apparatus.

An aspect of certain forms of the present technology is a medical device that is easy to use, e.g. by a person who does not have medical training, by a person who has limited dexterity, vision or by a person with limited experience in using this type of medical device.

An aspect of one form of the present technology is a portable RPT device that may be carried by a person, e.g., around the home of the person.

An aspect of one form of the present technology is a patient interface that may be washed in a home of a patient, e.g., in soapy water, without requiring specialised cleaning equipment. An aspect of one form of the present technology is a humidifier tank that may be washed in a home of a patient, e.g., in soapy water, without requiring specialised cleaning equipment.

The methods, systems, devices and apparatus described may be implemented so as to improve the functionality of a processor, such as a processor of a specific purpose computer, respiratory monitor and/or a respiratory therapy apparatus. Moreover, the described methods, systems, devices and apparatus can provide improvements in the technological field of automated management, monitoring and/or treatment of respiratory conditions, including, for example, sleep disordered breathing.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### 4 BRIEF DESCRIPTION OF THE DRAWINGS

The present technology is illustrated by way of example, and not by way of limitation, in the figures of the accompanying drawings, in which like reference numerals refer to similar elements including:

### 4.1 Respiratory Therapy Systems

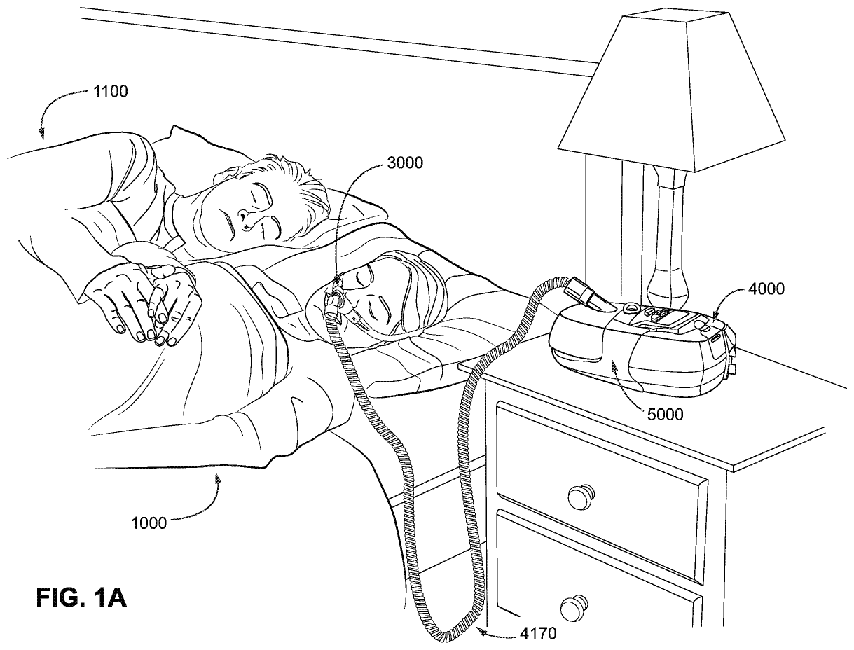

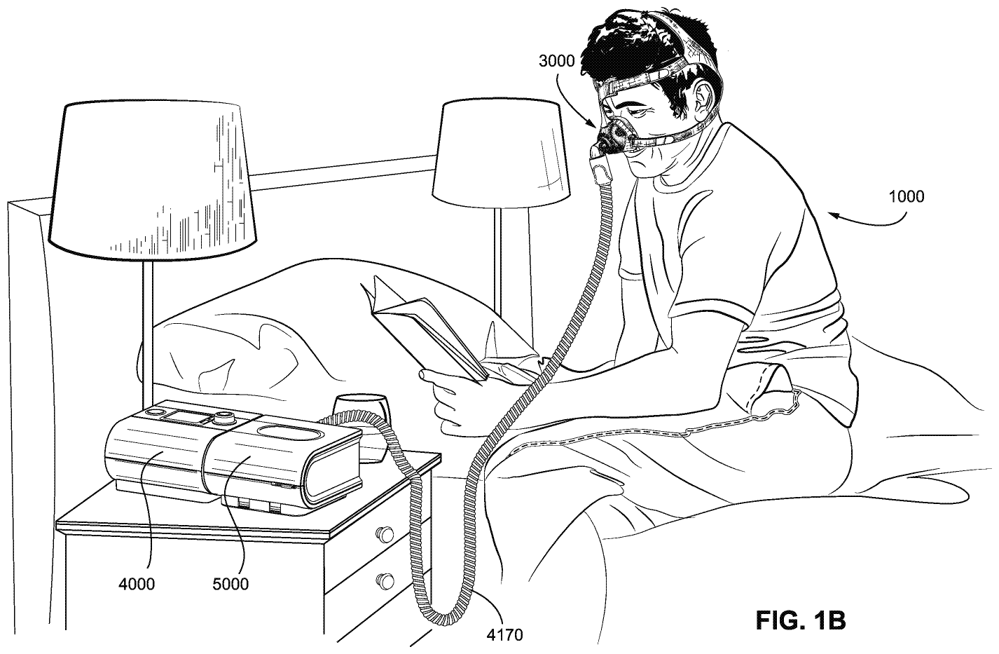

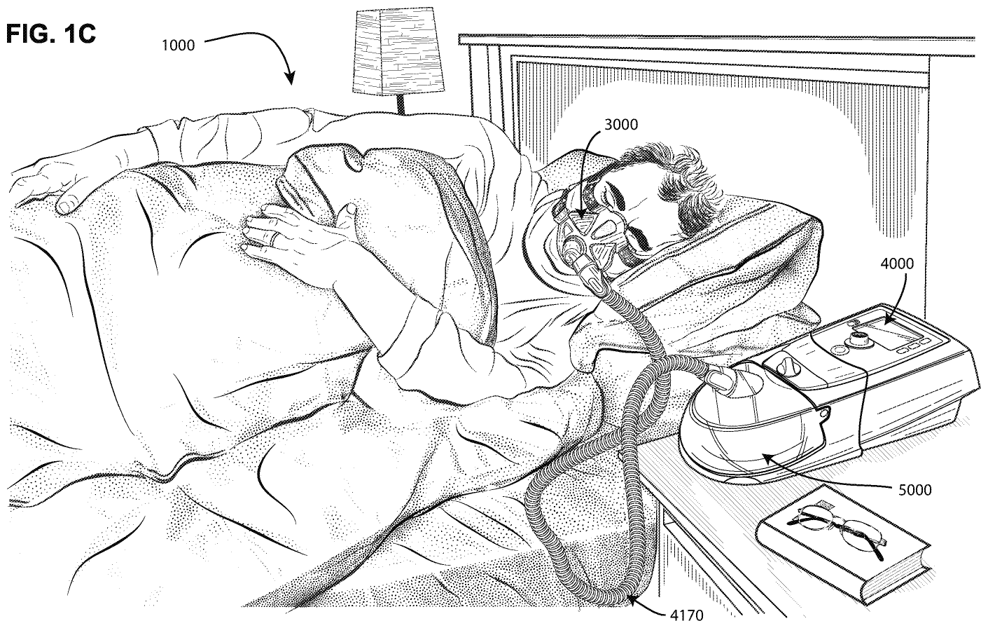

FIG. 1A shows a system including a patient 1000 wearing a patient interface 3000, in the form of nasal pillows, receiving a supply of air at positive pressure from an RPT device 4000. Air from the RPT device 4000 is humidified in a humidifier 5000, and passes along an air circuit 4170 to the patient 1000. A bed partner 1100 is also shown. The patient is sleeping in a supine sleeping position.

FIG. 1B shows a system including a patient 1000 wearing a patient interface 3000, in the form of a nasal mask, receiving a supply of air at positive pressure from an RPT device 4000. Air from the RPT device is humidified in a humidifier 5000, and passes along an air circuit 4170 to the patient 1000.

FIG. 1C shows a system including a patient 1000 wearing a patient interface 3000, in the form of a full-face mask, receiving a supply of air at positive pressure from an RPT device 4000. Air from the RPT device is humidified in a humidifier 5000, and passes along an air circuit 4170 to the patient 1000. The patient is sleeping in a side sleeping position.

### 4.2 Respiratory System and Facial Anatomy

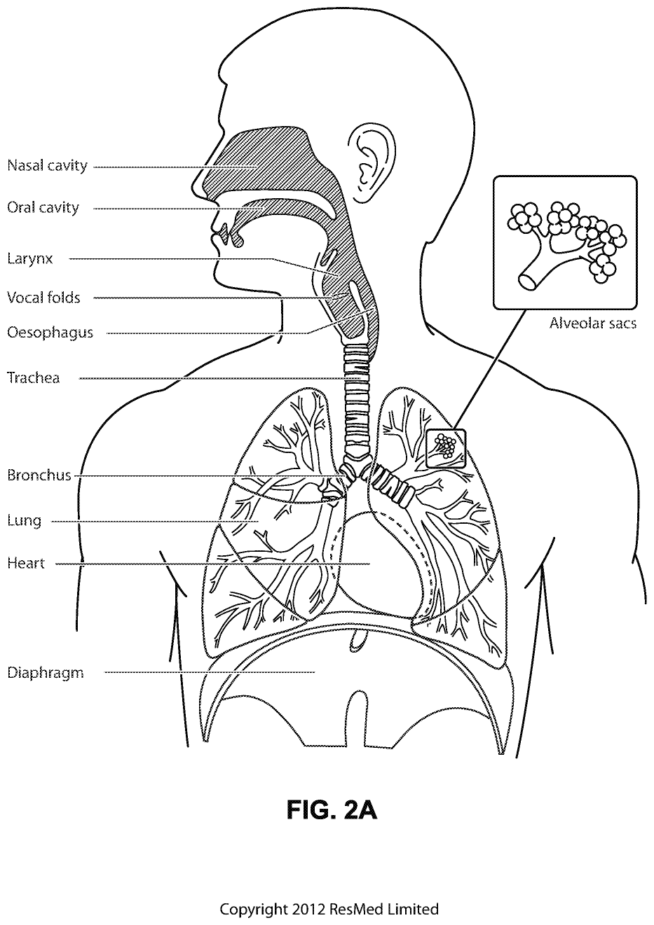

FIG. 2A shows an overview of a human respiratory system including the nasal and oral cavities, the larynx, vocal folds, oesophagus, trachea, bronchus, lung, alveolar sacs, heart and diaphragm.

### 4.3 Patient Interface

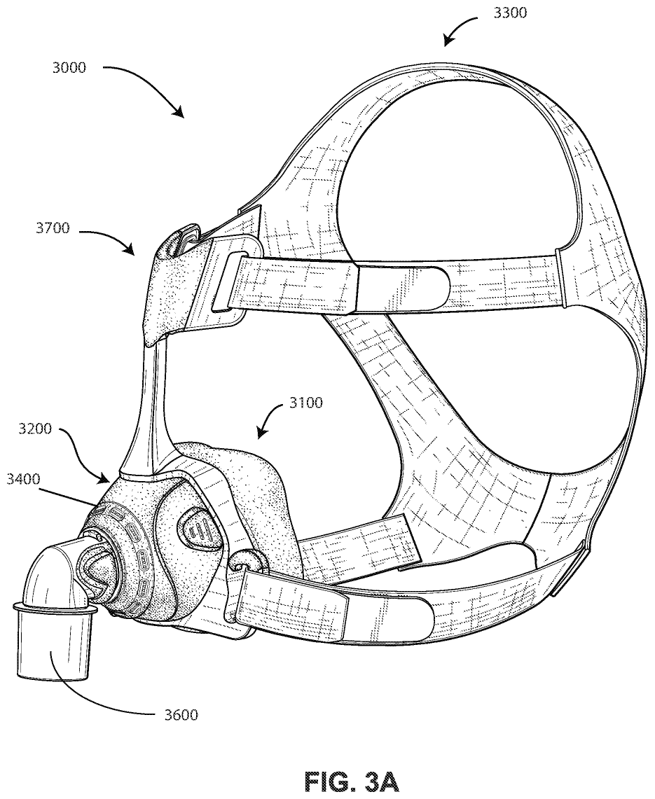

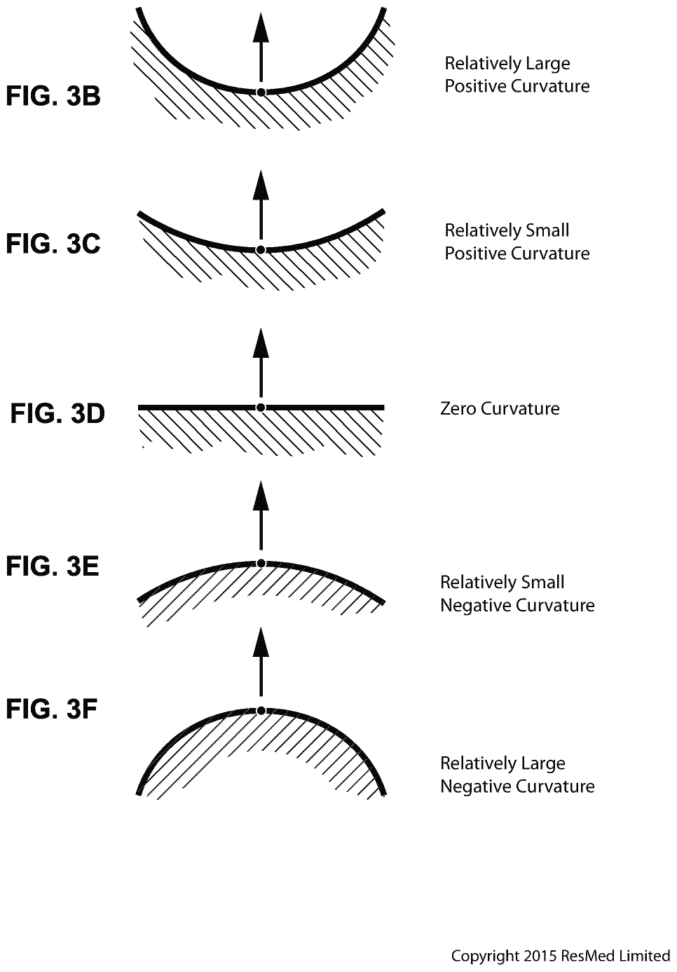

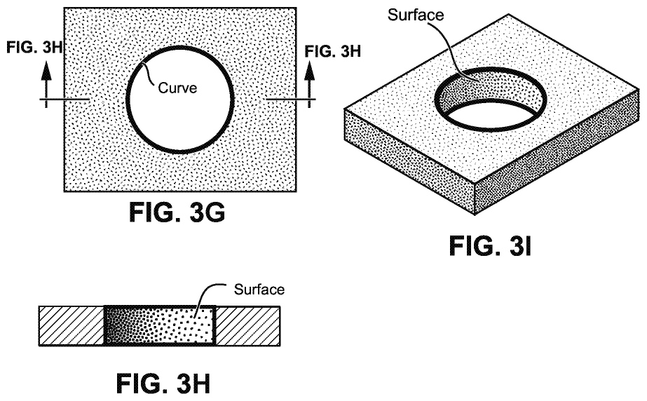

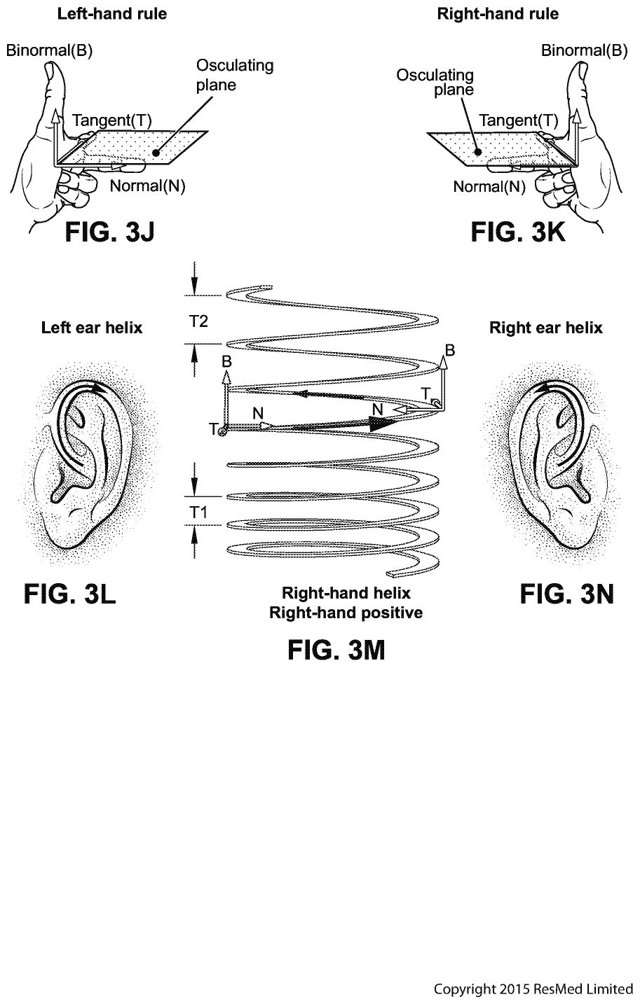

FIG. 3A shows a patient interface in the form of a nasal mask in accordance with one form of the present technology.

FIG. 3B shows a schematic of a cross-section through a structure at a point. An outward normal at the point is indicated. The curvature at the point has a positive sign, and a relatively large magnitude when compared to the magnitude of the curvature shown in FIG. 3C.

FIG. 3C shows a schematic of a cross-section through a structure at a point. An outward normal at the point is indicated. The curvature at the point has a positive sign, and a relatively small magnitude when compared to the magnitude of the curvature shown in FIG. 3B.

FIG. 3D shows a schematic of a cross-section through a structure at a point. An outward normal at the point is indicated. The curvature at the point has a value of zero.

FIG. 3E shows a schematic of a cross-section through a structure at a point. An outward normal at the point is indicated. The curvature at the point has a negative sign, and a relatively small magnitude when compared to the magnitude of the curvature shown in FIG. 3F.

FIG. 3F shows a schematic of a cross-section through a structure at a point. An outward normal at the point is indicated. The curvature at the point has a negative sign, and a relatively large magnitude when compared to the magnitude of the curvature shown in FIG. 3E.

FIG. 3G shows the surface of a structure, with a one dimensional hole in the surface. The illustrated plane curve forms the boundary of a one dimensional hole.

FIG. 3H shows a cross-section through the structure of FIG. 3G. The illustrated surface bounds a two dimensional hole in the structure of FIG. 3G.

FIG. 3I shows a perspective view of the structure of FIG. 3G, including the two dimensional hole and the one dimensional hole. Also shown is the surface that bounds a two dimensional hole in the structure of FIG. 3G.

FIG. 3J illustrates a left-hand rule.

FIG. 3K illustrates a right-hand rule.

FIG. 3L shows a left ear, including the left ear helix.

FIG. 3M shows a right ear, including the right ear helix.

FIG. 3N shows a right-hand helix.

### 4.4 RPT Device

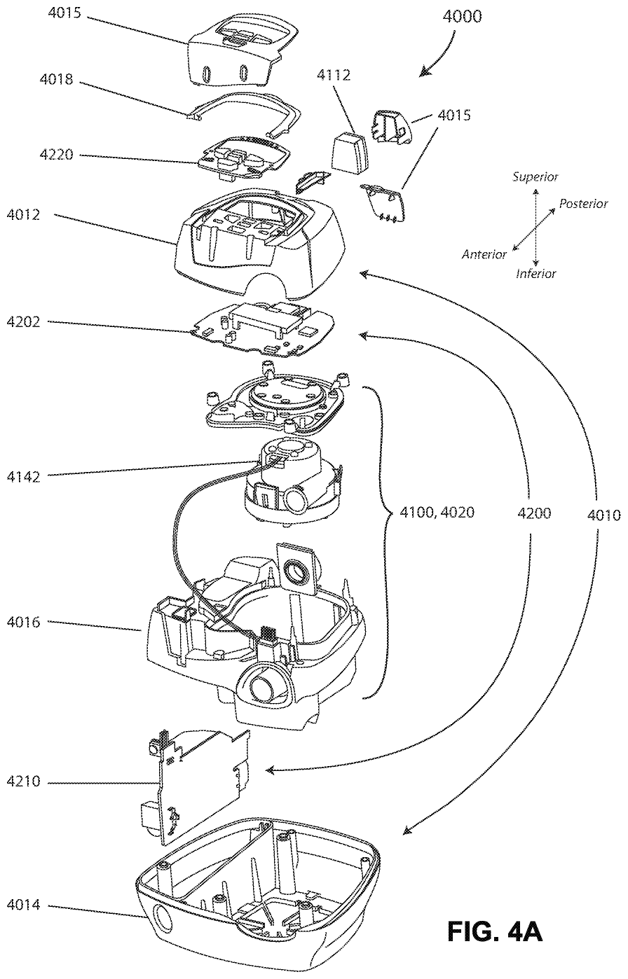

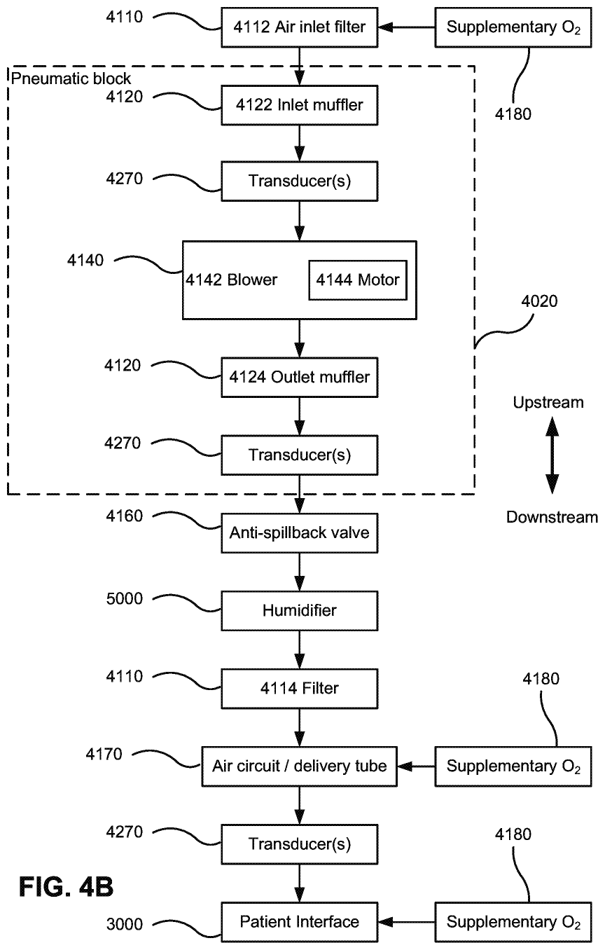

FIG. 4A shows an RPT device in accordance with one form of the present technology.

FIG. 4B is a schematic diagram of the pneumatic path of an RPT device in accordance with one form of the present technology. The directions of upstream and downstream are indicated with reference to the blower and the patient interface. The blower is defined to be upstream of the patient interface and the patient interface is defined to be downstream of the blower, regardless of the actual flow direction at any particular moment. Items which are located within the pneumatic path between the blower and the patient interface are downstream of the blower and upstream of the patient interface.

### 4.5 Humidifier

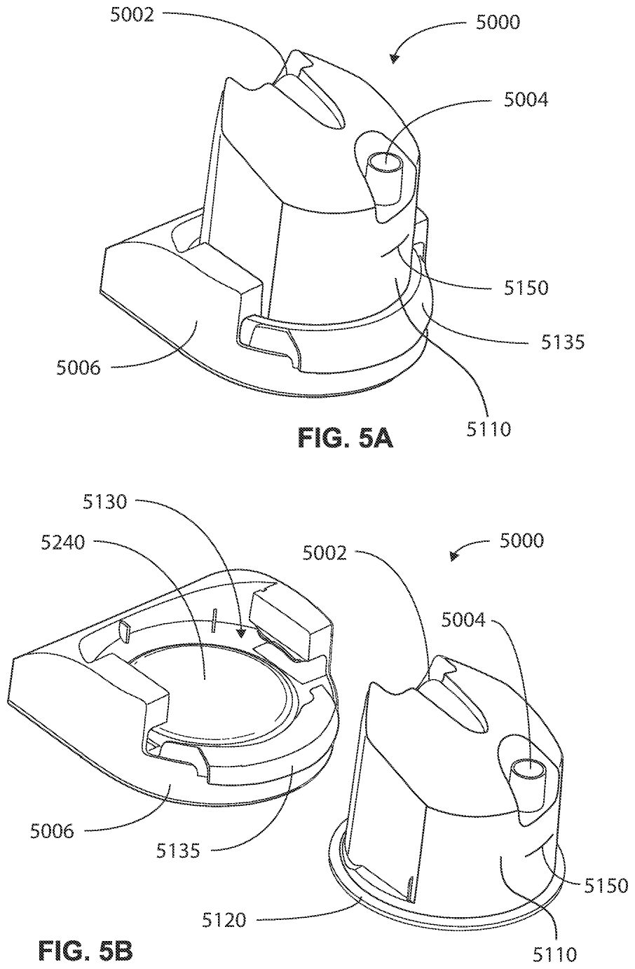

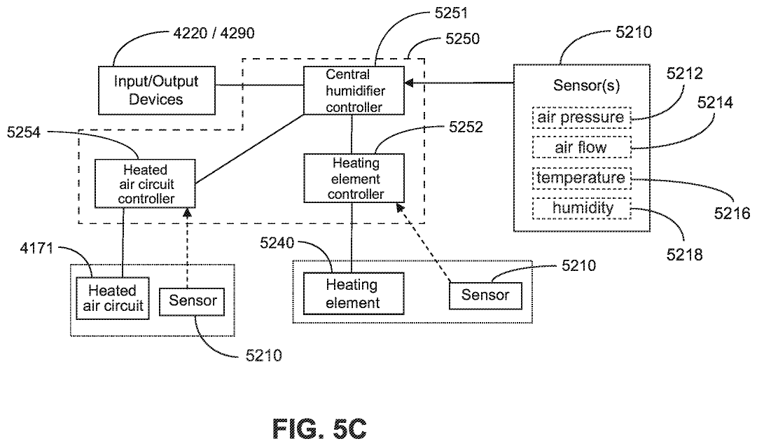

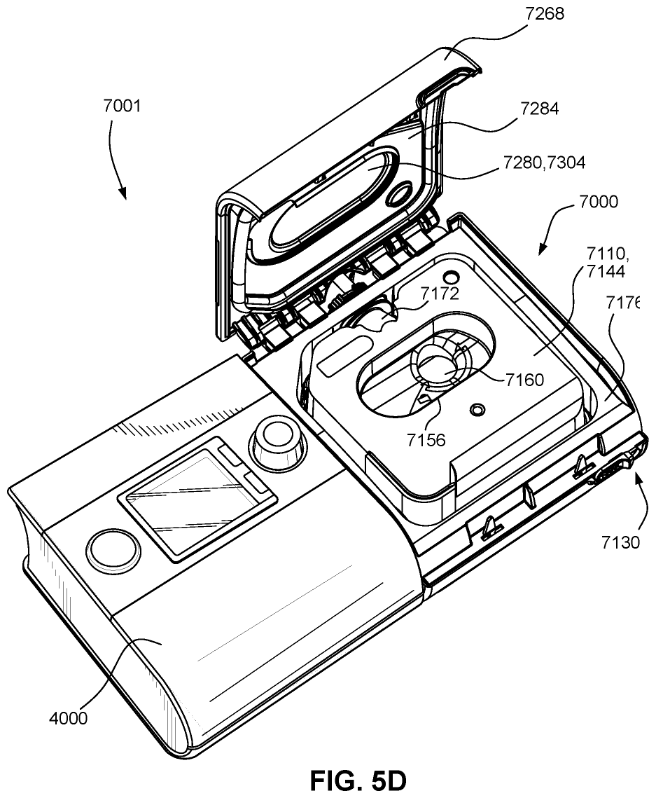

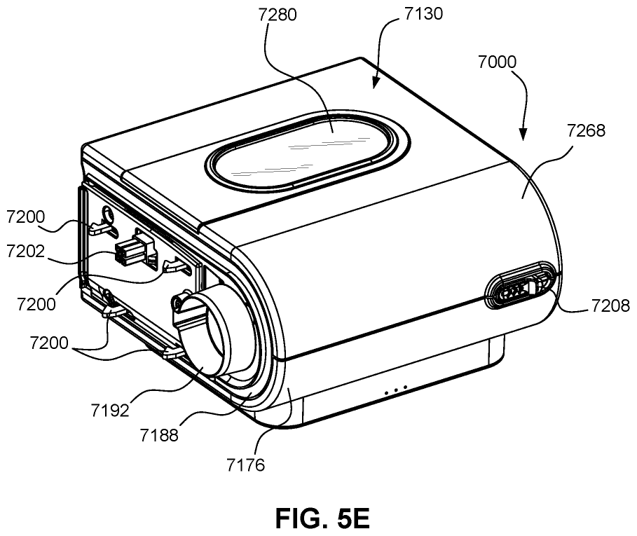

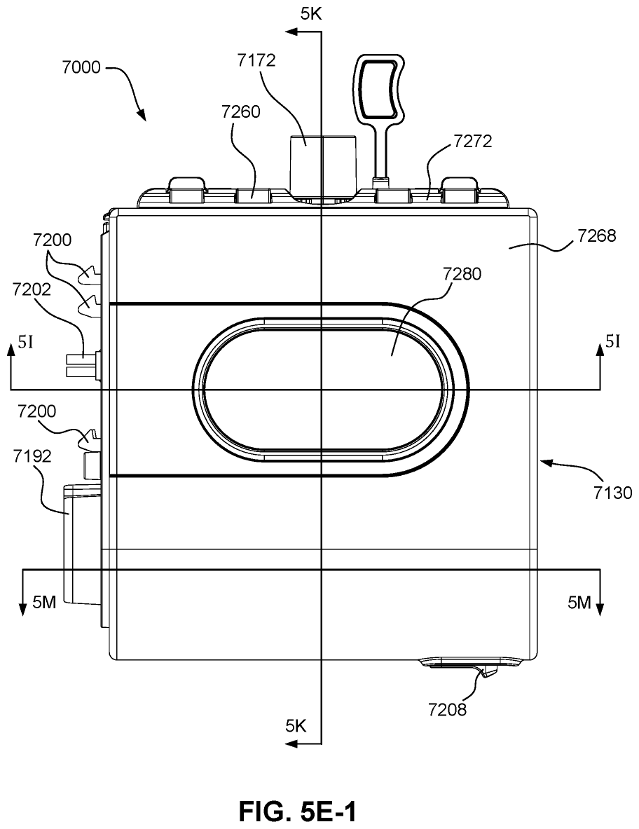

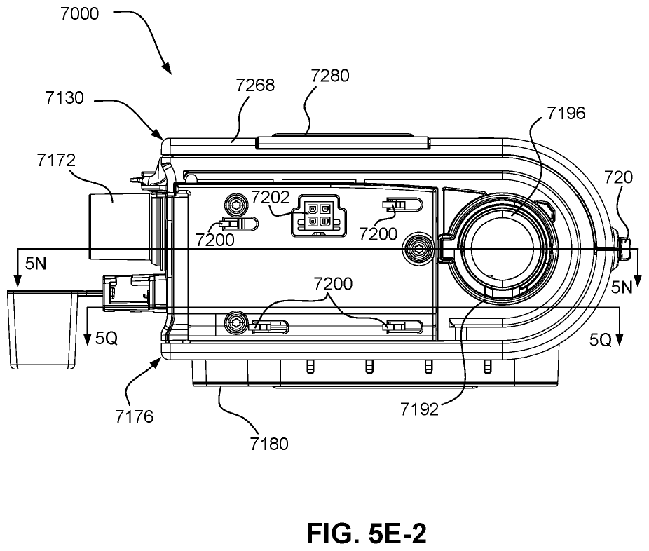

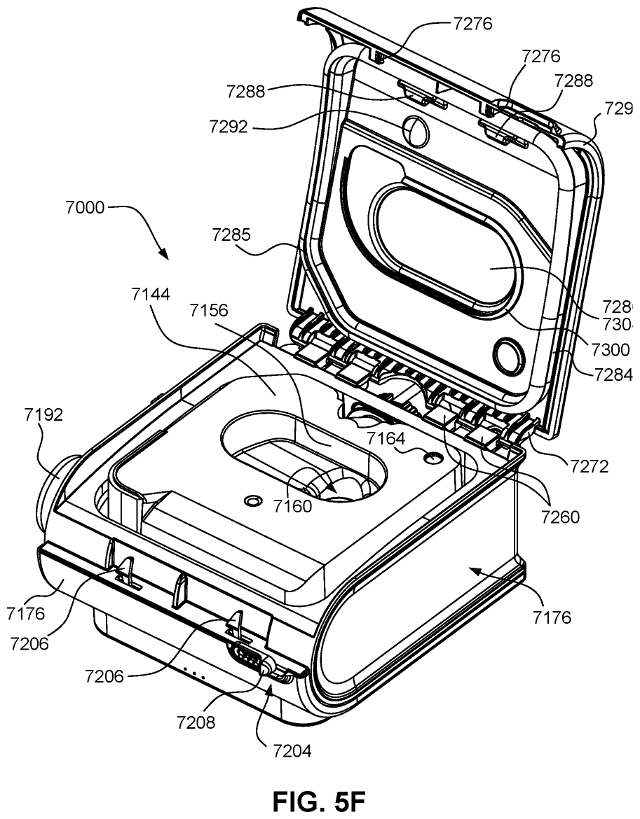

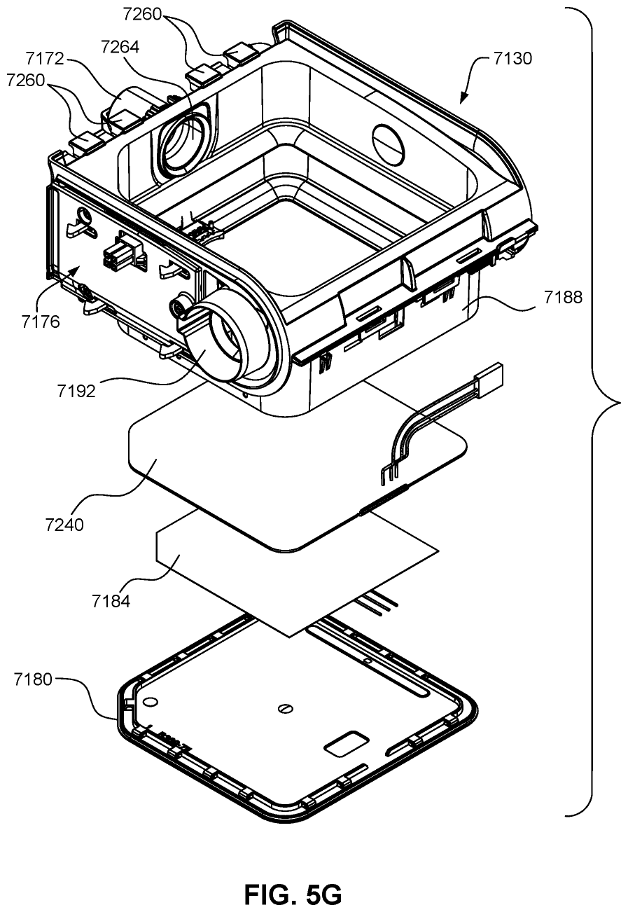

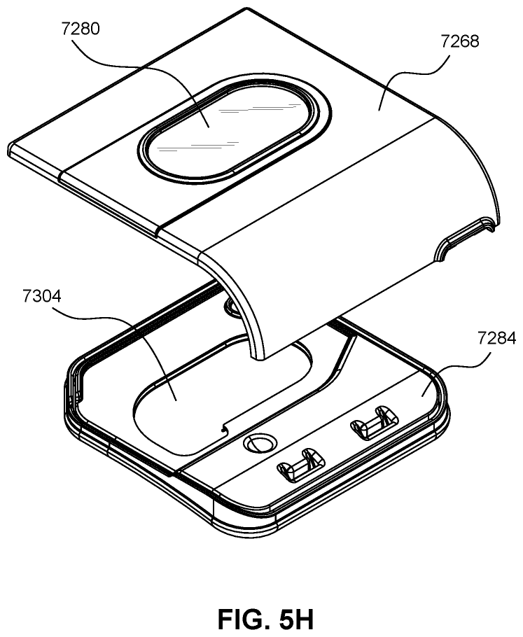

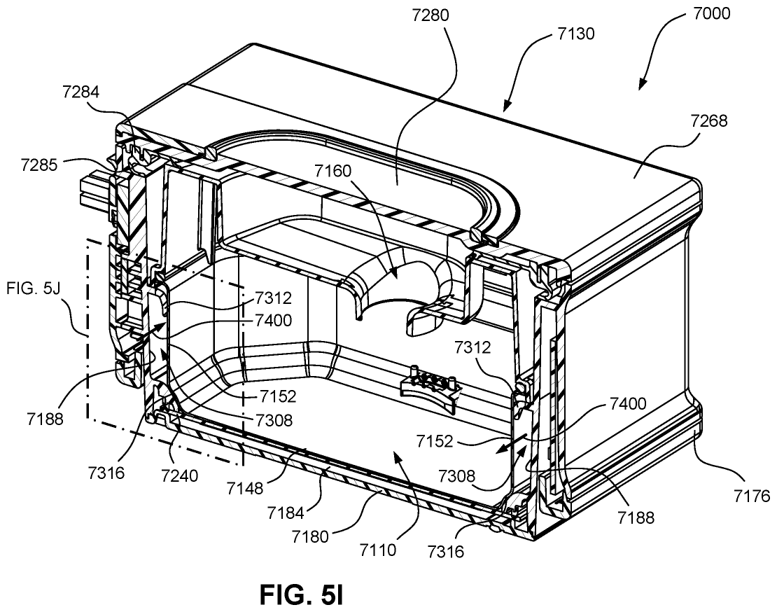

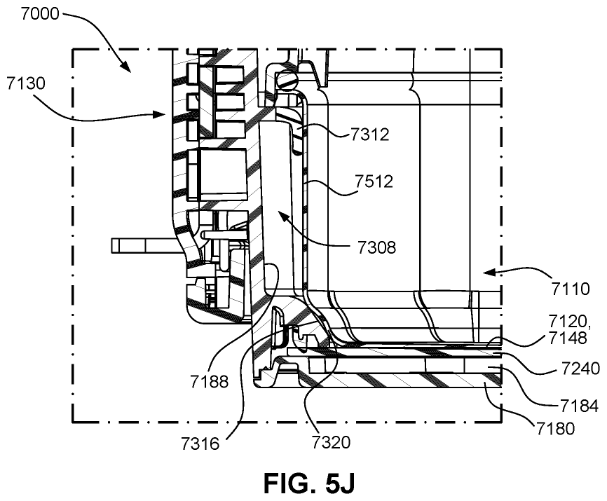

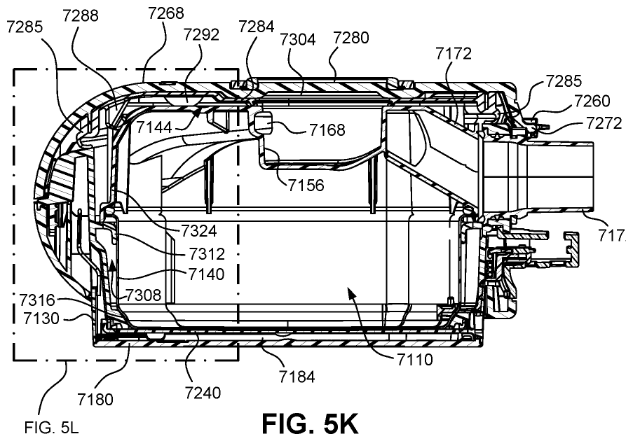

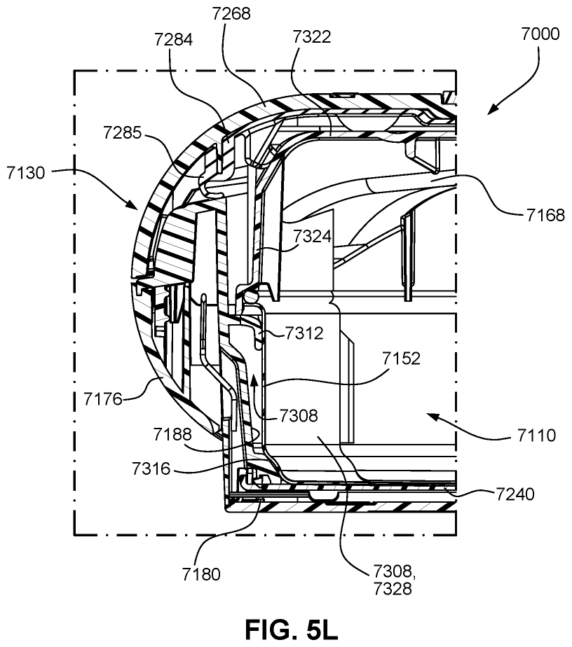

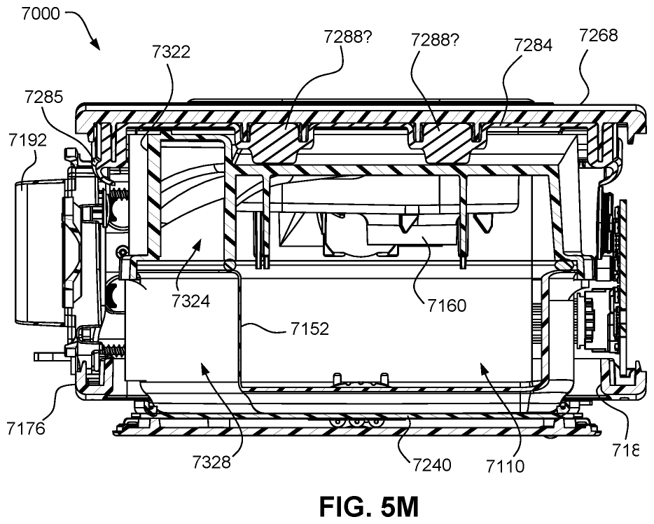

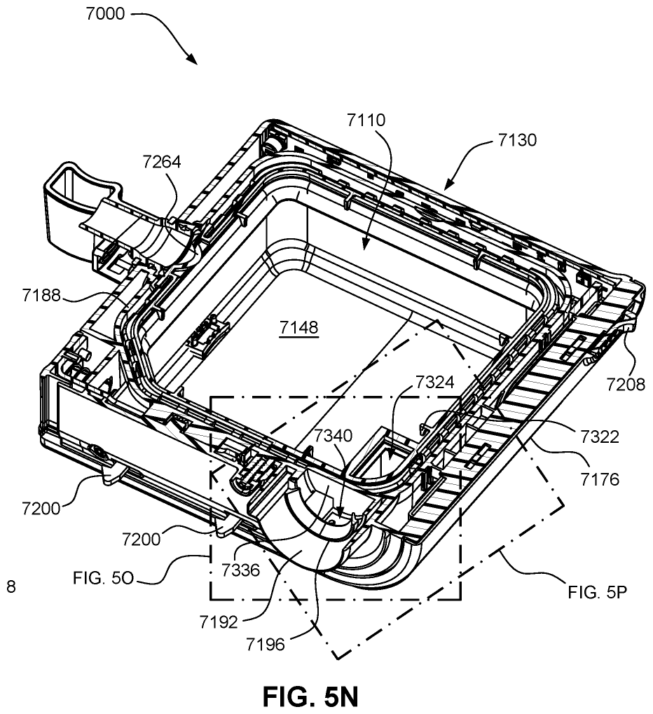

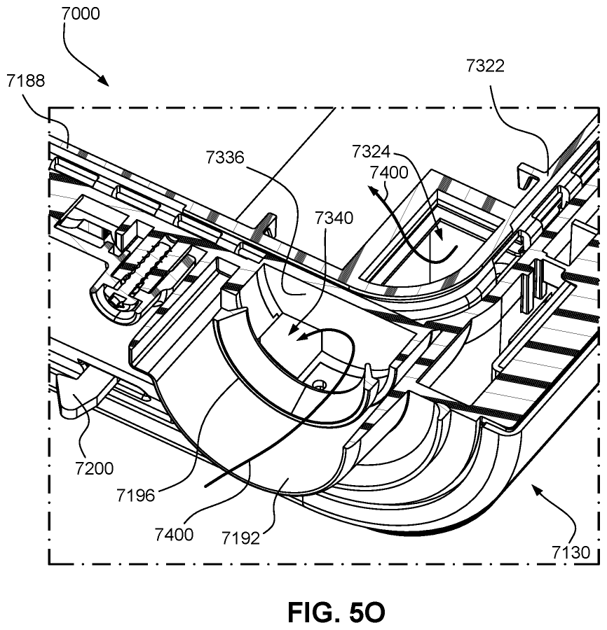

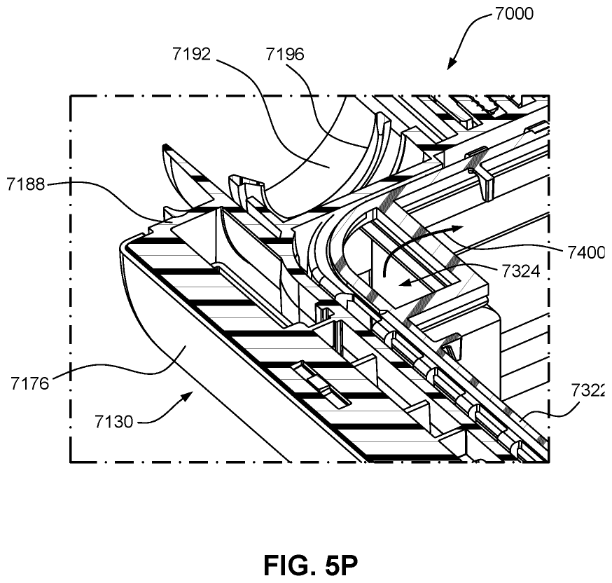

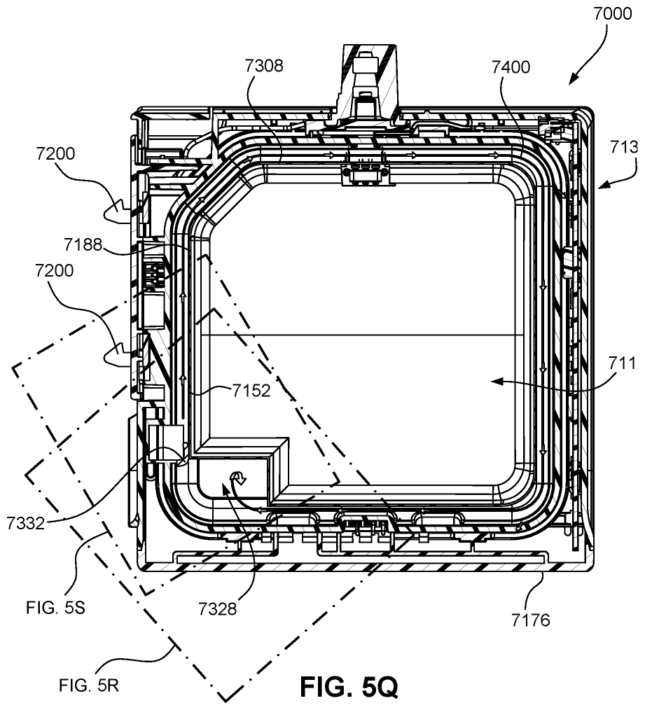

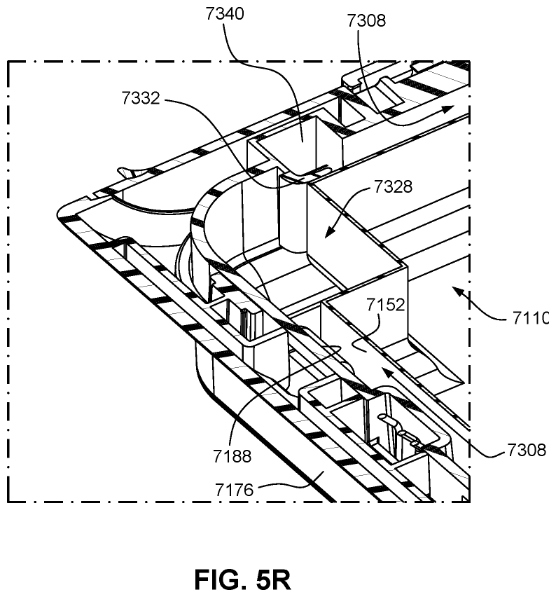

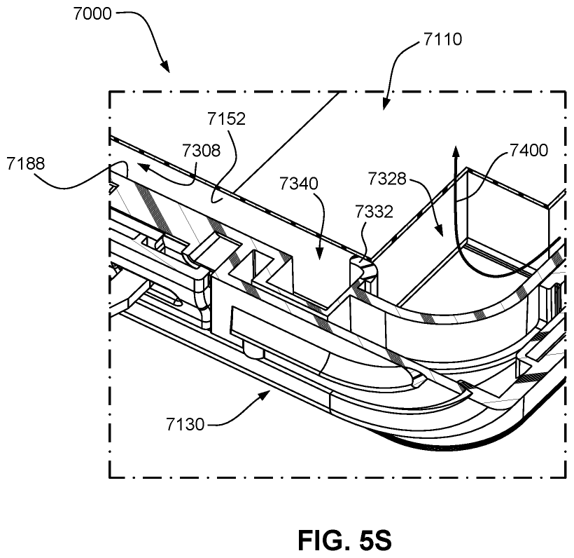

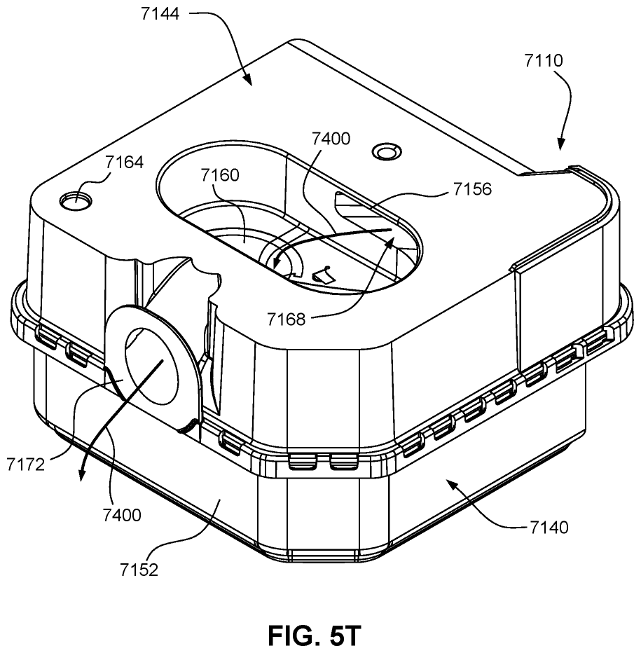

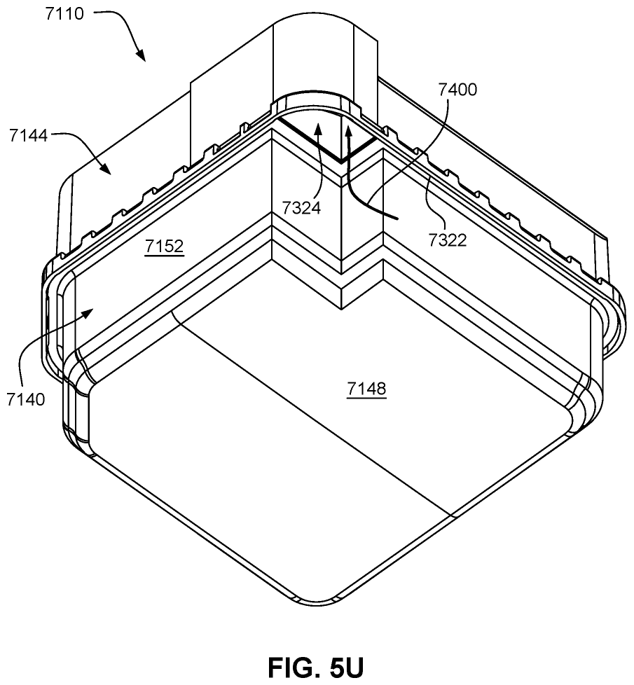

FIG. 5A shows an isometric view of a humidifier in accordance with one form of the present technology.

FIG. 5B shows an isometric view of a humidifier in accordance with one form of the present technology, showing a humidifier reservoir 5110 removed from the humidifier reservoir dock 5130.

FIG. 5C shows a schematic of a humidifier in accordance with one form of the present technology.

FIG. 5D shows a perspective view of a respiratory apparatus including a flow generator and humidifier in accordance with one form of the present technology.

FIG. 5E shows a perspective view of the humidifier of FIG. 5D with a lid in the closed position.

FIG. 5E-1 shows a top view of the humidifier of FIG. 5E.

FIG. 5E-2 shows a side view of the humidifier of FIG. 5E.

FIG. 5F shows a perspective view of the humidifier of FIG. 5D with the lid in the opened position.

FIG. 5G shows a partially exploded view of the humidifier of FIG. 5E.

FIG. 5H shows a perspective view of the lid of the humidifier of FIG. 5E, illustrating a seal exploded from the lid.

FIG. 5I shows a cross-sectional view of the humidifier of FIG. 5E-1 viewed along section I-I, illustrating a sealed air pathway between a water reservoir and a dock of the humidifier.

FIG. 5J shows a detail view of a respective cut-out from FIG. 5I.

FIG. 5K shows a cross sectional view of the humidifier of FIG. 5E-1 viewed along section K-K.

FIG. 5L shows a detail view of a respective cut-out FIG. 5K.

FIG. 5M shows a cross sectional view of FIG. 5E-1 viewed along section M-M, illustrating an entry chamber.

FIG. 5N shows a cross-sectional view of the humidifier of FIG. 5E-2 viewed along section N-N.

FIG. 5O shows a respective cut-out from FIG. 5N, illustrating a seal disposed within an inlet of the humidifier dock.

FIG. 5P shows a respective cut-out from FIG. 5N, illustrating a seal with an opening to the water reservoir.

FIG. 5Q shows a cross-sectional view of the humidifier of FIG. 5E-2 viewed along section Q-Q, illustrating a gas pathway extending substantially around a perimeter of the water reservoir.

FIG. 5R shows a detail perspective view of a respective cut-out from FIG. 5Q, illustrating an opening to the water reservoir form the gas pathway.

FIG. 5S shows a detail perspective view of a respective cut-out from FIG. 5Q, illustrating a bypass seal.

FIG. 5T shows a front perspective view of the water reservoir usable with the humidifier of FIG. 5E.

FIG. 5U shows a side perspective view of the water reservoir of FIG. 5T.

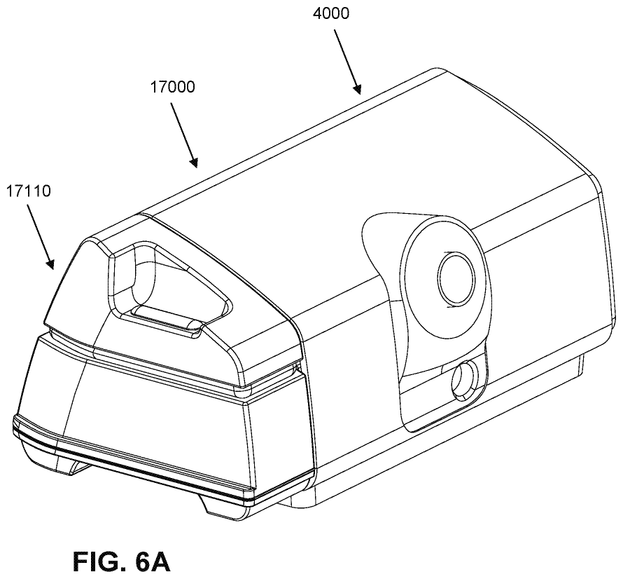

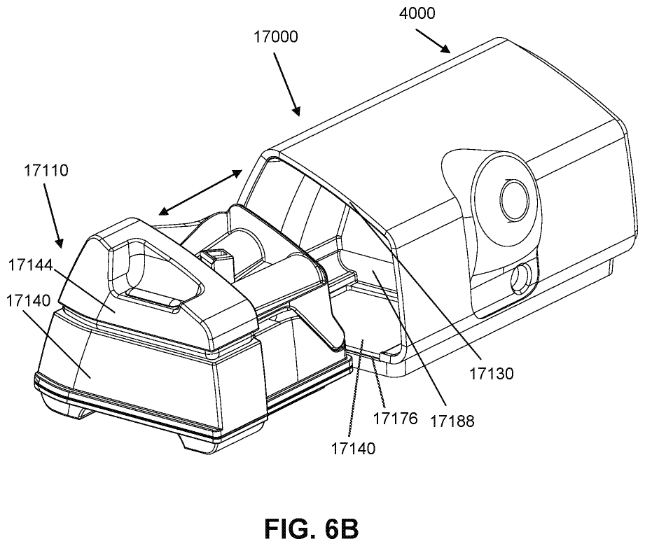

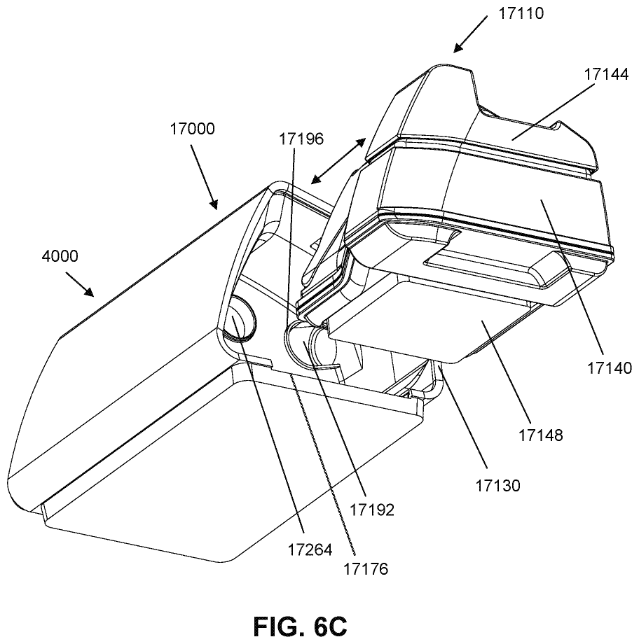

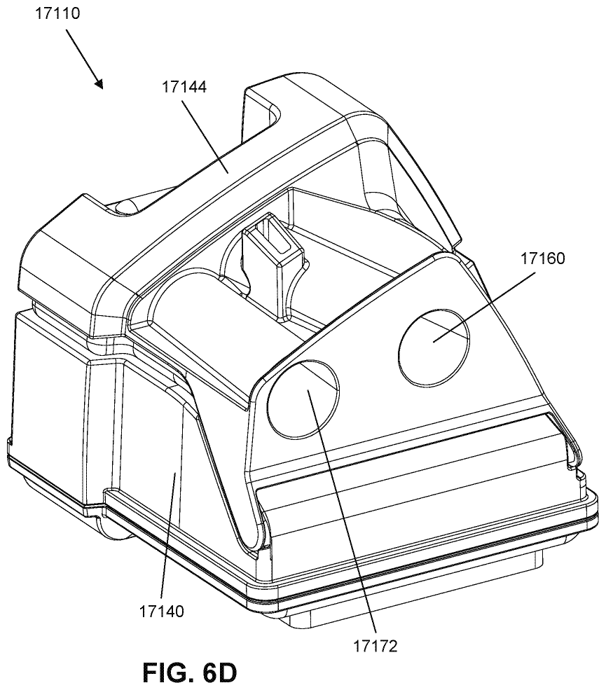

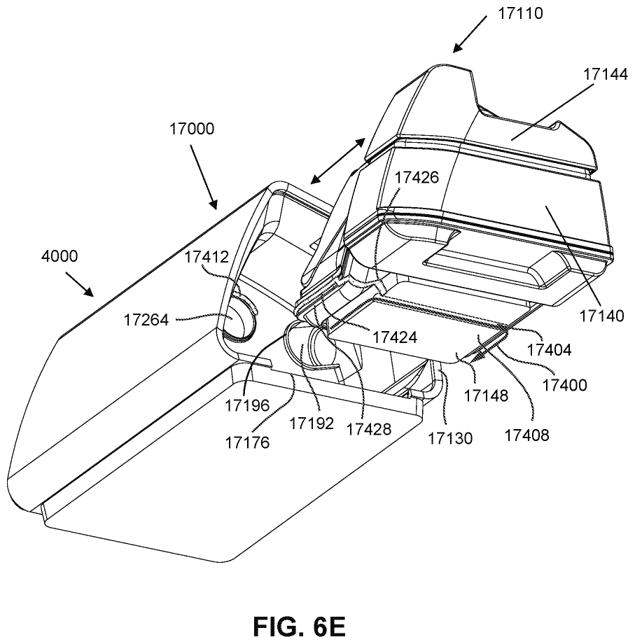

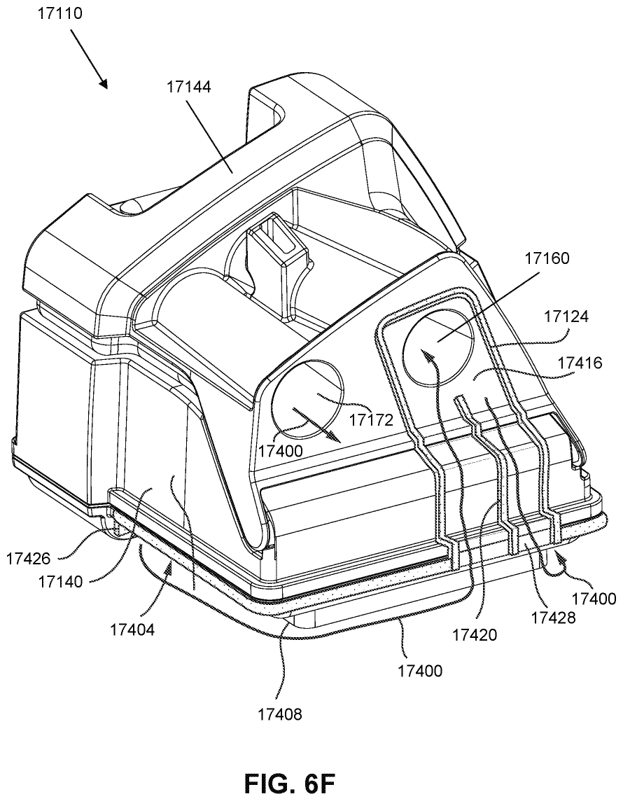

FIG. 6A shows a perspective view of a respiratory apparatus including a flow generator and humidifier in accordance with one form of the present technology.

FIG. 6B shows a partial top exploded view of the respiratory apparatus of FIG. 6A, illustrating a water reservoir removed from the humidifier.

FIG. 6C shows a partial bottom exploded view of the respiratory apparatus of FIG. 6A, illustrating a water reservoir removed from the humidifier.

FIG. 6D shows a perspective view of the water reservoir of FIG. 6B.

FIG. 6E shows a partial bottom perspective view of a respiratory apparatus of FIG. 6A, illustrating an alternate example of a water reservoir removed from the humidifier.

FIG. 6F shows a perspective view of the water reservoir of FIG. 6E.

### 4.6 Breathing Waveforms

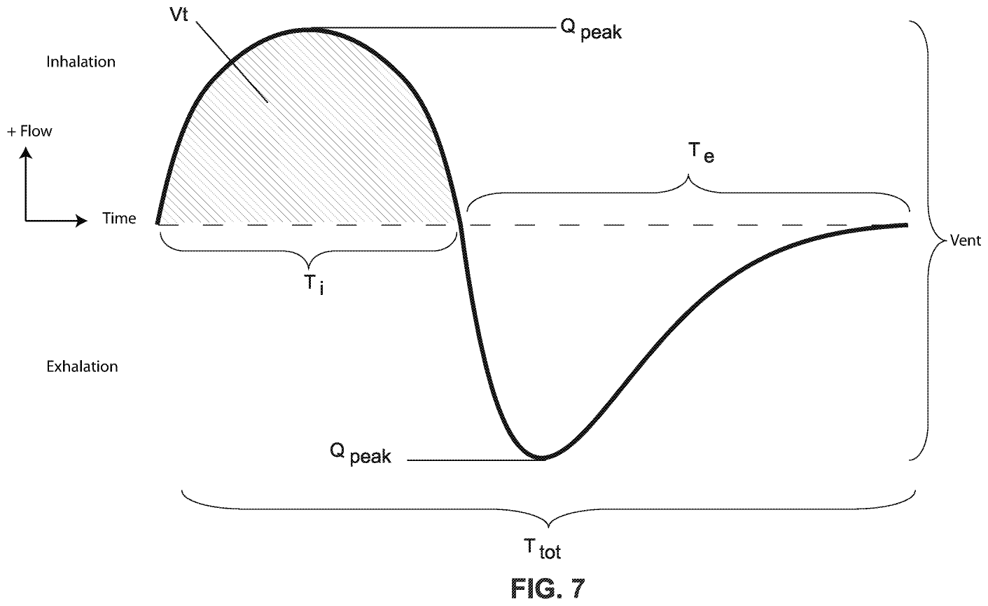

FIG. 7 shows a model typical breath waveform of a person while sleeping.

### 5 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

### 5.1 Therapy

In one form, the present technology comprises a method for treating a respiratory disorder comprising applying positive pressure to the entrance of the airways of a patient 1000.

In certain examples of the present technology, a supply of air at positive pressure is provided to the nasal passages of the patient via one or both nares.

In certain examples of the present technology, mouth breathing is limited, restricted or prevented.

### 5.2 Respiratory Therapy Systems

In one form, the present technology comprises a respiratory therapy system for treating a respiratory disorder. The respiratory therapy system may comprise an RPT device 4000 for supplying a flow of air to the patient 1000 via an air circuit 4170 and a patient interface 3000.

### 5.3 Patient Interface

A non-invasive patient interface 3000 in accordance with one aspect of the present technology comprises the following functional aspects: a seal-forming structure 3100, a plenum chamber 3200, a positioning and stabilising structure 3300, a vent 3400, one form of connection port 3600 for connection to air circuit 4170, and a forehead support 3700. In some forms a functional aspect may be provided by one or more physical components. In some forms, one physical component may provide one or more functional aspects. In use the seal-forming structure 3100 is arranged to surround an entrance to the airways of the patient so as to maintain positive pressure at the entrance(s) to the airways of the patient 1000. The sealed patient interface 3000 is therefore suitable for delivery of positive pressure therapy.

### 5.4 RPT Device

An RPT device 4000 in accordance with one aspect of the present technology comprises mechanical, pneumatic, and/or electrical components and is configured to execute one or more algorithms 4300, such as any of the methods, in whole or in part, described herein. The RPT device 4000 may be configured to generate a flow of air for delivery to a patient's airways, such as to treat one or more of the respiratory conditions described elsewhere in the present document.

In one form, the RPT device 4000 is constructed and arranged to be capable of delivering a flow of air in a range of −20 L/min to +150 L/min while maintaining a positive pressure of at least 6 cmH2O, or at least 10 cmH2O, or at least 20 cmH2O.

The RPT device may have an external housing 4010, formed in two parts, an upper portion 4012 and a lower portion 4014. Furthermore, the external housing 4010 may include one or more panel(s) 4015. The RPT device 4000 comprises a chassis 4016 that supports one or more internal components of the RPT device 4000. The RPT device 4000 may include a handle 4018.

The pneumatic path of the RPT device 4000 may comprise one or more air path items, e.g., an inlet air filter 4112, an inlet muffler 4122, a pressure generator 4140 capable of supplying air at positive pressure (e.g., a blower 4142), an outlet muffler 4124 and one or more transducers 4270, such as pressure sensors and flow rate sensors.

One or more of the air path items may be located within a removable unitary structure which will be referred to as a pneumatic block 4020. The pneumatic block 4020 may be located within the external housing 4010. In one form a pneumatic block 4020 is supported by, or formed as part of the chassis 4016.

The RPT device 4000 may have an electrical power supply 4210, one or more input devices 4220, a pressure generator 4140, and transducers 4270. Electrical components 4200 may be mounted on a single Printed Circuit Board Assembly (PCBA) 4202. In an alternative form, the RPT device 4000 may include more than one PCBA 4202.

### 5.4.1 RPT Device Mechanical & Pneumatic Components

An RPT device may comprise one or more of the following components in an integral unit. In an alternative form, one or more of the following components may be located as respective separate units.

### 5.4.1.1 Air Filter(s)

An RPT device in accordance with one form of the present technology may include an air filter 4110, or a plurality of air filters 4110.

In one form, an inlet air filter 4112 is located at the beginning of the pneumatic path upstream of a pressure generator 4140.

In one form, an outlet air filter 4114, for example an antibacterial filter, is located between an outlet of the pneumatic block 4020 and a patient interface 3000.

### 5.4.1.2 Muffler(s)

An RPT device in accordance with one form of the present technology may include a muffler 4120, or a plurality of mufflers 4120.

In one form of the present technology, an inlet muffler 4122 is located in the pneumatic path upstream of a pressure generator 4140.

In one form of the present technology, an outlet muffler 4124 is located in the pneumatic path between the pressure generator 4140 and a patient interface 3000.

### 5.4.1.3 Pressure Generator

In one form of the present technology, a pressure generator 4140 for producing a flow, or a supply, of air at positive pressure is a controllable blower 4142. For example, the blower 4142 may include a brushless DC motor 4144 with one or more impellers. The impellers may be located in a volute. The blower may be capable of delivering a supply of air, for example at a rate of up to about 120 litres/minute, at a positive pressure in a range from about 4 cmH2O to about 20 cmH2O, or in other forms up to about 30 cmH2O when delivering respiratory pressure therapy. The blower may be as described in any one of the following patents or patent applications the contents of which are incorporated herein by reference in their entirety: U.S. Pat. Nos. 7,866,944; 8,638,014; 8,636,479; and PCT Patent Application Publication No. WO 2013/020167.

The pressure generator 4140 may be under the control of the therapy device controller.

In other forms, a pressure generator 4140 may be a piston-driven pump, a pressure regulator connected to a high pressure source (e.g. compressed air reservoir), or a bellows.

### 5.4.1.4 Transducer(s)

Transducers may be internal of the RPT device, or external of the RPT device. External transducers may be located for example on or form part of the air circuit, e.g., the patient interface. External transducers may be in the form of non-contact sensors such as a Doppler radar movement sensor that transmit or transfer data to the RPT device.

In one form of the present technology, one or more transducers 4270 are located upstream and/or downstream of the pressure generator 4140. The one or more transducers 4270 may be constructed and arranged to generate signals representing properties of the flow of air such as a flow rate, a pressure or a temperature at that point in the pneumatic path.

In one form of the present technology, one or more transducers 4270 may be located proximate to the patient interface 3000.

In one form, a signal from a transducer 4270 may be filtered, such as by low-pass, high-pass or band-pass filtering.

### 5.4.1.5 Anti-Spill Back Valve

In one form of the present technology, an anti-spill back valve 4160 is located between the humidifier 5000 and the pneumatic block 4020. The anti-spill back valve is constructed and arranged to reduce the risk that water will flow upstream from the humidifier 5000, for example to the motor 4144.

### 5.4.2 RPT Device Electrical Components

### 5.4.2.1 Power Supply

A power supply 4210 may be located internal or external of the external housing 4010 of the RPT device 4000.

In one form of the present technology, power supply 4210 provides electrical power to the RPT device 4000 only. In another form of the present technology, power supply 4210 provides electrical power to both RPT device 4000 and humidifier 5000.

### 5.4.2.2 Input Devices

In one form of the present technology, an RPT device 4000 includes one or more input devices 4220 in the form of buttons, switches or dials to allow a person to interact with the device. The buttons, switches or dials may be physical devices, or software devices accessible via a touch screen. The buttons, switches or dials may, in one form, be physically connected to the external housing 4010, or may, in another form, be in wireless communication with a receiver that is in electrical connection to a central controller.

In one form, the input device 4220 may be constructed and arranged to allow a person to select a value and/or a menu option.

### 5.4.3 RPT Device Algorithms

As mentioned above, in some forms of the present technology, the central controller may be configured to implement one or more algorithms expressed as computer programs stored in a non-transitory computer readable storage medium, such as memory. The algorithms are generally grouped into groups referred to as modules.

In other forms of the present technology, some portion or all of the algorithms may be implemented by a controller of an external device such as the local external device or the remote external device. In such forms, data representing the input signals and/or intermediate algorithm outputs necessary for the portion of the algorithms to be executed at the external device may be communicated to the external device via the local external communication network or the remote external communication network. In such forms, the portion of the algorithms to be executed at the external device may be expressed as computer programs, such as with processor control instructions to be executed by one or more processor(s), stored in a non-transitory computer readable storage medium accessible to the controller of the external device. Such programs configure the controller of the external device to execute the portion of the algorithms.

In such forms, the therapy parameters generated by the external device via the therapy engine module (if such forms part of the portion of the algorithms executed by the external device) may be communicated to the central controller to be passed to the therapy control module.

### 5.5 Air Circuit

An air circuit 4170 in accordance with an aspect of the present technology is a conduit or a tube constructed and arranged to allow, in use, a flow of air to travel between two components such as RPT device 4000 and the patient interface 3000.

In particular, the air circuit 4170 may be in fluid connection with the outlet of the pneumatic block 4020 and the patient interface. The air circuit may be referred to as an air delivery tube. In some cases there may be separate limbs of the circuit for inhalation and exhalation. In other cases a single limb is used.

In some forms, the air circuit 4170 may comprise one or more heating elements configured to heat air in the air circuit, for example to maintain or raise the temperature of the air. The heating element may be in a form of a heated wire circuit, and may comprise one or more transducers, such as temperature sensors. In one form, the heated wire circuit may be helically wound around the axis of the air circuit 4170. The heating element may be in communication with a controller such as a central controller. One example of an air circuit 4170 comprising a heated wire circuit is described in U.S. Pat. No. 8,733,349, which is incorporated herewithin in its entirety by reference.

### 5.6 Humidifier

### 5.6.1 Humidifier Overview

In one form of the present technology there is provided a humidifier 5000 (e.g. as shown in FIG. 5A) to change the absolute humidity of air or gas for delivery to a patient relative to ambient air. Typically, the humidifier 5000 is used to increase the absolute humidity and increase the temperature of the flow of air (relative to ambient air) before delivery to the patient's airways.

The humidifier 5000 may comprise a humidifier reservoir 5110, a humidifier inlet 5002 to receive a flow of air, and a humidifier outlet 5004 to deliver a humidified flow of air. In some forms, as shown in FIG. 5A and FIG. 5B, an inlet and an outlet of the humidifier reservoir 5110 may be the humidifier inlet 5002 and the humidifier outlet 5004 respectively. The humidifier 5000 may further comprise a humidifier base 5006, which may be adapted to receive the humidifier reservoir 5110 and comprise a heating element 5240.

### 5.6.2 Humidifier Components

### 5.6.2.1 Water Reservoir

According to one arrangement, the humidifier 5000 may comprise a water reservoir or tub 5110 configured to hold, or retain, a volume of liquid (e.g. water) to be evaporated for humidification of the flow of air. The water reservoir 5110 may be configured to hold a predetermined maximum volume of water in order to provide adequate humidification for at least the duration of a respiratory therapy session, such as one evening of sleep. Typically, the reservoir 5110 is configured to hold several hundred millilitres of water, e.g. 300 millilitres (ml), 325 ml, 350 ml or 400 ml. In other forms, the humidifier 5000 may be configured to receive a supply of water from an external water source such as a building's water supply system.

According to one aspect, the water reservoir 5110 is configured to add humidity to a flow of air from the RPT device 4000 as the flow of air travels therethrough. In one form, the water reservoir 5110 may be configured to encourage the flow of air to travel in a tortuous path through the reservoir 5110 while in contact with the volume of water therein.

According to one form, the reservoir 5110 may be removable from the humidifier 5000, for example in a lateral direction as shown in FIG. 5A and FIG. 5B.

The reservoir 5110 may also be configured to discourage egress of liquid therefrom, such as when the reservoir 5110 is displaced and/or rotated from its normal, working orientation, such as through any apertures and/or in between its sub-components. As the flow of air to be humidified by the humidifier 5000 is typically pressurised, the reservoir 5110 may also be configured to prevent losses in pneumatic pressure through leak and/or flow impedance.

As shown in FIGS. 5T and 5S, a water reservoir 7110, such as one described in the U.S. Pat. No. 10,195,389, which is incorporated herewith by reference in its entirety, may include a tub base 7140, a tub lid 7144. The tub base 7140 and the tub lid 7144 may be coupled together, and may at least partially form a reservoir volume. In some forms, the tub base 7140 and the tub lid 7144 may be removably coupled together. The tub base 7140 and the tub lid 7144 may include complimentary features 7145, 7146 (e.g., mechanical latches) in order to detachably or removably couple the removable tub lid 7144 to the tub base 7140. In other forms, the tub base 7140 and the tub lid 7144 may be integrally formed, or otherwise fixed together.

In some forms, the tub base 7140 may include a substantially rectangular shape. For example, the illustrated tub base 7140 may include a substantially rectangular shape with at least one chamfered corner. In other examples, the tub base 7140 may have a partially rounded rectangular shape, with rounded instead of chamfered corners.

In other forms, the tub base 7140 may be another shape. For example, the tub base 7140 may have a rounded shape (e.g., circular or elliptical), where at least one side of the tub base 7140 may be arcuate (e.g., curved) instead of straight (e.g., like the illustrated example in FIGS. 5T and 5S).

In some forms, the tub base 7140 includes a tub bottom surface 7148 and at least one side wall 7152 extending from the tub bottom surface 7148. The side wall(s) 7152 at least partially form a volume of the tub base 7140. In some forms, the at least one side wall 7152 may be permanently connected to the tub bottom surface 7148. In some forms, the at least one side wall 7152 may be removably connected to the tub bottom surface 7148.

In certain forms, the at least one side wall 7152 may be oriented substantially perpendicularly with respect to the tub bottom surface 7148. The side wall(s) 7152 may be considered vertical side walls, and may extend substantially upwardly (e.g., in a superior direction), when the tub base 7140 is resting on the tub bottom surface 7148.

In certain forms, the at least one side wall 7152 may be inclined relative to the tub bottom surface 7148. In other words, an angle between the side wall(s) 7152 and the tub bottom surface 7148 may not be 90°. In some forms, the side wall(s) 7152 may project over the tub bottom surface 7148. In other words, the angle between the tub bottom surface 7148 and the side surface(s) 7152 as measured from the bottom surface 7148 may be less than 90°.

In one form, multiple side walls 7152 may be connected to the tub bottom surface 7148. Each side wall 7152 may have the same angle relative to the tub bottom surface 7148. An area of an opening formed between the free ends of the side walls 7152 may be less than an area of the tub bottom surface 7148 as a result of the inclined side walls 7152.

In other forms, the side wall(s) may project away from the tub bottom surface 7148. In other words, the angle between the tub bottom surface 7148 and the side wall(s) 7152 as measured from the tub bottom surface 7148 may be greater than 90°.

In one form, multiple side walls 7152 may be connected to the tub bottom surface 7148. Each side wall 7152 may have the same angle relative to the tub bottom surface 7148. An area of an opening formed between the free ends of the side walls 7152 may be greater than an area of the tub bottom surface 7148 as a result of the inclined side walls 7152.

In some forms, the tub lid 7144 is coupled to the at least one side wall 7152 of the tub base 7140, and may be substantially opposite to the tub bottom surface 7148.

In certain forms, the tub lid 7144 may be oriented substantially perpendicularly with respect to the side wall(s) 7152 (e.g., when coupled to the side walls 7152). In other words, the tub lid 7144 and the tub bottom surface 7148 may be substantially parallel to one another when the tub lid 7144 is in a closed position.

In some forms, the tub lid 7144 includes an opening 7156, which may provide communication into the reservoir volume when the tub lid 7144 is coupled to the tub base 7140. For example, in forms where the tub lid 7144 is removable from the tub base 7140, the user may add additional liquid (e.g., pour additional water) into the reservoir volume through the opening 7156. The opening 7156 may provide a large opening that may assist in minimizing spilling when liquid is added to the water reservoir 7110.

In some forms, the opening 7156 may have an oval or elliptical shape. For example, the opening 7156 may include a rounded shape (e.g., the opening 7156 may not have corners) and may be longer in one direction than in other.

In some forms, the opening 7156 may have a generally rounded shape. In some forms, the opening 7156 may or may not include an elongated shape (e.g., the opening 7156 may include a substantially circular shape).

In other forms, the opening 7156 may include a different shape that is not entirely rounded. For example, the opening 7156 may include a triangular, rectangular, or any other similar shape. In some forms, these other shapes may have angled corners and may not have a rounded section. In other forms, these other shapes may be partially rounded (e.g., they may include rounded corners).

In certain forms, the opening 7156 includes an outlet 7160, which may be a smaller opening disposed within the larger perimeter of the opening 7156. The outlet 7160 may provide direct communication with the reservoir volume of the tub base 7140.

Liquid added to the opening 7156 may be directed into the tub base 7140 through the outlet 7160. As described above, a patient may pour liquid into the larger opening 7156 in order to limit spillage. The liquid introduced by the patient into the opening 7156 may then be directed toward the outlet 7160 (e.g., by a sloped surface). The liquid may then enter the water reservoir 7110 through the outlet 7160.

The outlet 7160 may also allow pressurized breathable gas to enter the reservoir volume of the tub base 7140. In other words, pressurized breathable gas supplied by the RPT device 4000 may flow though the outlet 7160 in order to reach the heated liquid, before proceeding to the patient's airways with increased humidity (e.g., acquired while within the water reservoir 7110).

In some forms, the tub lid 7144 includes a tub emptying aperture 7164, which may be spaced apart from the outlet 7160. For example, the tub emptying aperture 7164 may be spaced apart from the opening 7156.

In certain forms, the tub emptying aperture 7164 may have a smaller diameter than the outlet 7160. In some examples, the patient may pour liquid from the tub base 7140 out through the tub emptying aperture 7164. In some examples, the patient may pour liquid out through the outlet 7160, and air may pass through the tub emptying aperture 7164 in order to create a more uniform flow through the outlet 7160.

In certain forms, the tub emptying aperture 7164 may be sealed while the humidifier 7000 is in use, so that air is limited from exiting the water reservoir 7110 through the tub emptying aperture 7164.

In some forms, the tub lid 7144 may include a channel 7168 that receives the flow of pressurized breathable gas generated by the RPT device 4000 that enters the humidifier 7000. The channel 7168 may direct the pressurized breathable gas toward the liquid in the interior of the tub base 7140. For example, the channel 7168 may be connected to the opening 7156. The channel 7168 may direct and/or convey the pressurized breathable gas toward the opening 7156, and into the tub base 7140 through the outlet 7160.

In some forms, water reservoir 7110 includes an outlet 7172 for the humidified flow of pressurized breathable gas. The now humidified air may exit the water reservoir 7110 through the outlet 7172, before continuing toward the patient (e.g., through a tube configured to deliver the humidified flow to a patient interface, like a mask).

### 5.6.2.2 Heating Element

A heating element 5240 may be provided to the humidifier 5000 in some cases to provide a heat input to one or more of the volume of water in the humidifier reservoir 5110 and/or to the flow of air. The heating element 5240 may comprise a heat generating component such as an electrically resistive heating track. One suitable example of a heating element 5240 is a layered heating element such as one described in the PCT Patent Application Publication No. WO 2012/171072, which is incorporated herewith by reference in its entirety.

In some forms, the heating element 5240 may be provided in the humidifier base 5006 where heat may be provided to the humidifier reservoir 5110 primarily by conduction as shown in FIG. 5B.

The heating element 7240 of humidifier 7000 may be substantially similar to the heating element 5240.

### 5.6.2.3 Conductive Portion

According to one arrangement, the reservoir 5110 comprises a conductive portion 5120 configured to allow efficient transfer of heat from the heating element 5240 to the volume of liquid in the reservoir 5110. In one form, the conductive portion 5120 may be arranged as a plate, although other shapes may also be suitable. All or a part of the conductive portion 5120 may be made of a thermally conductive material such as aluminium (e.g. approximately 2 mm thick, such as 1 mm, 1.5 mm, 2.5 mm or 3 mm), another heat conducting metal or some plastics. In some cases, suitable heat conductivity may be achieved with less conductive materials of suitable geometry.

In some forms, the tub bottom surface 7148 of the tub base 7140 may include a conductive portion 7120 in order to assist in conductive heat transfer from a heating element 7240 to liquid stored in the tub base 7140.

In certain forms, the entire tub bottom surface 7148 may be constructed from a conductive material (e.g., metal). This may provide the largest contact area with a heating element 7240, and may allow for the greatest amount of heat transfer to the liquid within the water reservoir 7110.

In certain forms, a portion of the tub bottom surface 7148 may be constructed partially from a conductive material (e.g., metal), and partially from an insulative material (e.g., plastic). The insulative material may allow for some heat transfer, but may be more insulative as compared to the conductive material. The conductive portion 7120 may contact a similarly sized heating element 7240. The smaller heating element 7240 may require less electrical energy than a larger heating element 7240.

In either of the above described forms, the heating element 7240 may have a smaller surface area than the tub bottom surface 7148. This may be equal to the surface area of the conductive portion of the tub bottom surface 7148, or it may be less than the surface area of the conductive portion.

Alternatively, the heating element 7240 may be the same size as the tub bottom surface 7240.

### 5.6.2.4 Humidifier Reservoir Dock

In some forms, the humidifier 5000 may comprise a humidifier reservoir dock or humidifier chamber 5130 (see e.g., FIG. 5B) configured to receive the humidifier reservoir 5110.

In some arrangements, the chamber 5130 may comprise a locking feature such as a locking lever 5135 configured to retain the reservoir 5110 in the chamber 5130. The locking lever 5135 may be exposed while chamber 5130 receives the humidifier reservoir 5110. The locking lever 5135 may limit the humidifier reservoir 5110 from moving out of the chamber 5130 in the superior and/or lateral (e.g., left/right) directions. A user may actuate the locking lever 5135 (e.g., by pressing in the inferior direction) in order to enable movement of the humidifier reservoir 5110 out of the humidifier chamber 5130. The locking lever 5135 may be biased into a locked position (e.g., position that limits movement of the humidifier reservoir 5110), so the patient may require two hands in order to remove the humidifier reservoir 5110 (e.g., one to keep the locking lever 5135 in an unlocked position and one to remove the humidifier reservoir 5110).

### 5.6.2.4.1 Chamber Base

As shown in FIG. 5D-5G, some forms of the chamber 7130 may include a chamber base 7176. The chamber base 7176 may removably receive the water reservoir 7110 (although in other examples, the water reservoir 7110 may be integrally formed with the chamber base 7176). The chamber base 7176 may have a similar size and shape (e.g., substantially rectangular) as the water reservoir 7110. The chamber base 7176 may have a larger volume (e.g., a longer length, a longer width, and/or a larger depth) in order to receive the water reservoir 7110.

The chamber base 7176 may include a chamber bottom surface 7180. As shown in FIG. 5G, the chamber bottom surface 7180 may be a substantially planar (e.g., flat) surface. When received within the chamber 7130, the water reservoir 7110 may rest on the chamber bottom surface 7180.

In certain forms, the heating element 7240 may be coupled to the chamber bottom surface 7180 so that the heating element 7240 may contact the tub bottom surface 7148 (e.g., the conductive portion 7120).

In certain forms, an insulation layer 7184 may be provided between the heating element 7240 and the chamber bottom surface 7180. In other words, the heating element 7240 may not be in direct contact with the chamber bottom surface 7180. The insulation layer 7184 may limit heat transferring toward the chamber bottom surface 7180, and instead may direct heat produced by the heating element 7240 toward the water reservoir 7110.

This may assist with convective heat transfer, as the insulation layer 7184 may trap or contain heat produced by the heating element 7240 within the chamber 7130. Heat may circulate throughout the chamber 7130 and provide additional heating (e.g., in addition to the conductive heating between the heating element 7240 and the conductive portion 7120). This may increase the efficiency of the humidifier 7000, as less heat is can escape the chamber 7130 without providing a benefit (e.g., heating the liquid in the water reservoir 7110).

The insulation layer 7184 may enable a patient to pick up the chamber 7130 (e.g., by the chamber bottom surface 7180). The insulation layer 7184 may limit conductive heat transfer from the heating element 7240 to the chamber bottom surface 7180. This may prevent the chamber bottom surface 7180 from becoming too hot for a patient to touch (or too hot for the surface on which the chamber bottom surface 7180 rests). The insulation layer 7184 may allow a patient to pick up and/or reposition the chamber 7130 while the humidifier 7000 is in use.

In some forms, at least one chamber side wall 7188 is coupled to the chamber bottom surface 7180. The chamber side wall(s) 7188 at least partially form a volume of the chamber base 7176. The RPT device 4000 (e.g., fan or other flow generator) may be disposed outside of the volume of the chamber base 7176. In other words, the water reservoir 7110 may be received within the volume (e.g., within the chamber side wall(s) 7188, but the RTP device 4000 is positioned outside of the chamber side wall(s) 7188).

In certain forms, the chamber side wall(s) 7188 may be substantially perpendicular with respect to the chamber bottom surface 7180. In other words, the chamber side wall(s) may be substantially vertical, and extend upwardly (e.g., in a superior direction) from the chamber bottom surface 7180 while the humidifier 7000 is in use (e.g., when the chamber bottom surface 7180 faces a support surface, like a table).

In other forms, the chamber side wall(s) may be inclined relative to the chamber bottom surface 7180. In other words, an angle between the side wall(s) 7152 and the tub bottom surface 7148 may not be 90°. In some forms, the side wall(s) 7152 may project over the tub bottom surface 7148. In other words, the angle between the tub bottom surface 7148 and the side surface(s) 7152 as measured from the bottom surface 7148 may be less than 90°.

In one form, multiple side walls 7152 may be connected to the tub bottom surface 7148. Each side wall 7152 may have the same angle relative to the tub bottom surface 7148. An area of an opening formed between the free ends of the side walls 7152 may be less than an area of the tub bottom surface 7148 as a result of the inclined side walls 7152.

In other forms, the side wall(s) may project away from the tub bottom surface 7148. In other words, the angle between the tub bottom surface 7148 and the side wall(s) 7152 as measured from the tub bottom surface 7148 may be greater than 90°.

In one form, multiple side walls 7152 may be connected to the tub bottom surface 7148. Each side wall 7152 may have the same angle relative to the tub bottom surface 7148. An area of an opening formed between the free ends of the side walls 7152 may be greater than an area of the tub bottom surface 7148 as a result of the inclined side walls 7152

In some forms, a chamber opening 7192 is disposed in the at least one chamber side wall 7188. The chamber opening 7192 may receive the flow of pressurized breathable gas generated by the RPT device 4000. In other words, the flow of pressurized breathable gas may enter the volume of the chamber base 7176 after passing through the chamber opening 7192, and continuing to flow downstream toward the water reservoir 7110. The pressurized breathable gas entering the chamber opening 7192 may be substantially un-humidified (e.g., as compared to the humidity of breathable gas inhaled by the patient using a patient interface 3000 connected to the humidifier 7000).

In certain forms, the chamber opening 7192 may assist in directing the flow of pressurized breathable gas from the RPT device 4000 toward the heated liquid in the water reservoir 7110. For example, the chamber opening 7192 may direct the flow of pressurized breathable gas in a direction that leads toward the outlet 7160.

In certain forms (see e.g., FIG. 5E), the chamber opening 7192 is substantially circular, although other shapes (e.g., elliptical, triangular, rectangular, etc.) may also be used. The chamber opening 7192 may also project from the at least one chamber side wall 7188. The chamber opening 7192 may be received by the RPT device 4000 when the humidifier 7000 is connected to the RPT device 4000.

In certain forms, a sealing member or chamber wall seal 7196 is disposed proximate to the chamber opening 7192. The sealing member 7196 may assist in limiting the flow of pressurized breathable gas from escaping into the ambient while traveling between the RPT device 4000 and the humidifier 7000. In other words, the sealing member 7196 may maintain a pressurized environment between the RPT device 4000 and the humidifier 7000.

As shown in FIG. 5O, a seal 7196 (e.g., a ring seal) is disposed within the chamber opening 7192. An outlet of the RPT device 4000 may engage the seal 7196 when the RPT device 4000 and the humidifier 7000 are coupled together. The seal 7196 may be compressed when the RPT device 4000 and the humidifier 7000 are coupled together, and may seal against an inner surface of the chamber opening 7192.

As shown in FIGS. 5E to 5E-2, some forms of the chamber 7130 include connectors 7200 that may engage corresponding recesses (not shown) in the RPT device 4000. The connectors 7200 may selectively retain the chamber 7130 to the RPT device 4000 while in use by the patient.

In certain forms, the connectors 7200 are latches (e.g., spring-biased latches). The latches 7200 may be ordinarily biased toward a locked position, and may be selectively movable toward an unlocked position by the user in order to disconnect the chamber 7130 from the RPT device 4000.

In certain forms, the latches 7200 are disposed on the same face of the chamber 7130 as the chamber opening 7192. In other words, the latches 7200 and the chamber opening 7192 may be disposed on the same chamber side wall 7188.

In some forms, the chamber 7130 includes an electrical connector 7202. The electrical connector 7202 may receive electrical power from the RPT device 4000 in order to power electrical components of the humidifier 7000 (e.g., the heating element 7240).

In certain forms, the electrical connector 7202 is disposed proximate to the latches 7200. For example, the electrical connector 7202 may be disposed on the same chamber side wall 7188 as the latches 7200. Thus, the chamber 7130 may be electrically and mechanically connected to the RPT device 4000. The connection between the RPT device 4000 and the humidifier 7000 may form a respiratory apparatus 7001.

In other examples, the RPT device 4000 and the chamber 7130 may be integrally formed with one another so that the humidifier 7000 cannot be removed from the RPT device 4000. For example, the chamber 7130 may be integrated into the RPT device 4000 so that the chamber 7130 cannot be separated from the RPT device 4000 (e.g., the respiratory apparatus 7001 in FIG. 5D may not be separable). In certain forms, the water reservoir 7110 may still be removable from the chamber 7130 (e.g., in a vertical direction), even though the chamber 7130 is integrally formed with the RPT device 4000. When the chamber 7130 and the RPT device 4000 are integrated, there may still be a partition between the chamber 7130 and the RPT device 4000. In other words, airflow may exit the RPT device 4000 and enter the chamber 7130 through the chamber opening 7192. The volume of the chamber base 7176 may still be at least partially formed by chamber walls 7188, outside of which is positioned the RPT device 4000, so that the flow of pressurized breathable gas flows downstream through the chamber opening 7192 in order to enter the volume of the chamber base 7176.

In still other examples illustrated in FIGS. 6A-6D, the RPT device 4000 may be integrated with a humidifier 17000, which is similar to the humidifier 7000. The chamber 17130 may include a chamber base 17176 and at least one chamber side wall 17188, which together may at least partially form the chamber volume. The chamber side wall 17188 may separate the RPT device 4000 from the humidifier 17000, so that a flow generator of the RPT device 4000 is not disposed within the chamber volume. The heater plate 17140 may be disposed on the chamber base 17176 within the chamber volume.

Unlike the humidifier 7000, the water reservoir 17110 may be inserted into the chamber 17130 of the humidifier 17000 in a lateral (e.g., horizontal direction), instead of a vertical direction. As shown in FIG. 6C, an operational position of the humidifier 17000 illustrates the chamber 17130 opening in the lateral direction. The water reservoir 17110 may include a similar orientation as the water reservoir 7110, in that the surface 17148 (e.g., constructed from a thermally conductive material) contacting the heating element 17240 may be an inferior surface in use. Additionally, the tub lid 17144 may also pivot relative to the tub base 17140 about a horizontal axis. Alternatively, the tub lid 17144 may be slidable relative to the tub base 17140 in order to move between an open and a closed position. For example, the tub lid 17144 may be completely removed in the tub base 17144 in the open position.

As the water reservoir 17110 is inserted into the chamber 17130 so that a tub inlet 17160 (see e.g., FIG. 6D) is aligned with the chamber outlet 17264, and a tub outlet 17172 (see e.g., FIG. 6D) is aligned with a chamber inlet 17192. Seals (e.g., seal 17196) may be disposed proximate to at least one of the openings, in order to form a substantially sealed pathway between the RPT device 4000 and the water reservoir 17110.

However, in some forms, the seals may not completely seal around the openings (e.g., the seal 17196 does not completely seal against the chamber inlet) or the seals may be omitted (e.g., the chamber outlet 17264 is spaced apart from the tub inlet 17160 when the water reservoir 17110 is fully inserted into the chamber 17130). The water reservoir 17110 may seal against an opening to the chamber 17130 (e.g., using a seal or gasket formed between the edge of the chamber that abuts a shoulder of the water reservoir) but the pressurized air may be able to flow through the chamber 17130, and at least partially around the heated based of the water reservoir 17110. The flow of pressurized breathable gas may be able to increase in temperature (i.e., pre-heated) as a result of heat given off by the heating element 17240 (e.g., convective heat transfer). The flow of pressurized breathable gas may travel at least partially around the chamber, or at least partially around the base of the chamber, passively picking up or scavenging heat from the heating element 17240 that would otherwise be wasted. The air may then be guided (by way of respective edges and sealing gaskets) to enter the water reservoir 17110 with an increased temperature than when it left the RPT device 4000 and passed through the chamber inlet 17192. A seal may remain between the chamber inlet 17192 and the tub outlet 17172 so that the air cannot exit the chamber 17130 without first entering the water reservoir 17110.

As shown in FIGS. 6E and 6F, seals may be positioned in the chamber 17130 and on the water reservoir 17110 in order to allow the flow of pressurized breathable gas to flow at least partially around a perimeter of the water reservoir 17110 in order to be pre-heated before entering the water reservoir 17110.

As shown in FIG. 6E, the water reservoir 17110 may include a groove 17404 through the surface 17148. In the illustrated example, the groove 17404 extends along a straight, linear path entirely from one side of the surface 17148 to another. For example, the groove 17404 may intersect lateral sides 17408 of the surface 17148. The lateral sides 17408 may be parallel to the insertion direction of the water reservoir 17110 into the chamber 17130.

In other examples, the shape of the groove 17404 may be different than the illustrated form. For example, the groove 17404 may follow a curved path between the lateral sides 17408. This may allow the groove 17404 to have a longer length.

In still other examples, the groove 17404 may extend at least partially obliquely with respect to the lateral sides 17408. In other words, the groove 17404 may not intersect the lateral sides 17408 perpendicularly. This variation may be applied to either of the forms described above (i.e., the linear groove 17404 or the curved groove 17404).

In some forms, the entire surface 17148 may be formed with a heat conductive material (e.g., metal). Thus, the groove 17404 may also be formed with the heat conductive material. Although the surface of the groove 17404 may not contact the heating element 17240, the groove 17404 may still allow for heat transfer (e.g., convective heat transfer) as a result of the thermally conductive material.

As shown in FIG. 6F, seals may be connected to the water reservoir 17110 in order to direct airflow 17400 through the chamber 17130 before entering the tub inlet 17160. This may allow for the flow of pressurized breathable gas to pre-heat before entering the water reservoir 17110.

In other forms, the seals may be at least partially connected within the chamber 17130 and positioned to contact the water reservoir 17110 upon insertion. For example, a deflector seal 17412 may be included within the chamber 17130 proximate to the chamber outlet 17264 (see e.g., FIG. 6E). For example, the deflector seal 17412 may positioned around at least a superior portion of the chamber outlet 17264. The deflector seal 17412 may also at least partially intersect the chamber outlet 17264 (e.g., angled in an inferior and/or anterior direction relative to the chamber outlet 17264). The deflector seal 17412 may serve to deflect airflow 17400 exiting the chamber outlet 17264 toward an inferior direction. This may cause the flow of pressurized air to flow into the chamber 17130 instead of directly into the tub inlet 17160.

Returning to FIG. 6F, the water reservoir 17110 may include a sealing surface 17416 that may contact the deflector seal 17412. The sealing surface 17416 may be inferior to the tub inlet 17160. When fully inserted, the deflector seal 17412 may contact and seal against the sealing surface 17416. Airflow 17400 exiting the chamber outlet 17264 may be initially blocked from entering the tub inlet 17160, and may instead be directed in an inferior direction toward a bottom of the chamber 17130 (e.g., toward the heating element 17240).

In some forms, the deflector seal 17412 may taper as it projects from the from the water reservoir 17110 toward the sealing surface 17416. For example, the deflector seal 17412 may have a maximum width greater than or equal to the diameter of the chamber outlet 17264, and may have a minimum width less than the diameter of the tub inlet 17160. The deflector seal 17412 may also contact that sealing surface 17416 off-center from the tub inlet 17160. In other examples, the deflector seal 17412 may be centered with the tub inlet 17160, and/or may include a continuous width.

In some forms, a dividing gasket 17420 may be connected to the sealing surface 17416. The dividing gasket 17420 may be positioned within the extended width of the tub inlet 17160. In other words, the dividing gasket 17420 is spaced apart from the tub inlet 17160, but radially within the width of the tub inlet 17160.

In some forms, the dividing gasket 17420 may have a greater length than a width. For example, the dividing gasket 17420 may extend further in a superior-inferior direction (e.g., along the sealing surface 17416 between the tub inlet 17160 and the surface 17148) than in a lateral direction (e.g., along the sealing surface 17416 between the lateral sides 17408). In some forms, the width (e.g., along the lateral direction) may be less than the diameter of the tub inlet 17160.

The water reservoir 17100 may also include at least one guiding gasket or pathway seal 17424. The pathway seal 17424 may form a sealed portion around at least a portion of the perimeter of the water reservoir 17110. In the illustrated example, the pathway seal 17424 is radially outside of the tub inlet 17160 so that the tub inlet 17424 is included within the sealed perimeter.

The pathway seal 17424 may also extend around at least a portion of an inferior region of the water reservoir 17110. Like in the humidifier 7000, the pathway seal 17424 may at least partially form a sealed pathway for the flow of pressurized gas around at least part of the perimeter of the water reservoir 17110.

In the illustrated example, the pathway seal 17424 may extend along the sealing surface 17416 for a location superior to the tub inlet 17160 to a location inferior to the tub inlet 17160 (e.g., a skirt 17428 of the water reservoir 17110 adjacent to the surface 17148). The pathway seal 17424 may then form an upper portion of the pathway around a base of the water reservoir 17110.

The pathway seal 17424 may direct the flow of pressurized breathable gas along at least a portion of the perimeter of the water reservoir 17110. In the illustrated example, the pathway seal 17424 may direct the airflow 17400 through the groove 17404. For example, the pathway seal 17424 may connect to the water reservoir (or alternatively within the chamber 17130) along the lateral sides 17408 at the openings of the groove 17404. An end portion 17426 of the pathway seal 17424 may also extend away from each lateral side 17408 (e.g., perpendicular to the lateral sides 17408) in order to block flow out of the chamber 17130. In other words, the pathway seal 17424 may limit fluid flow along the lateral sides 17408 in a direction away from the RPT device 4000 and past the entrance or exit to the groove 17404.

The dividing gasket 17420 may interact with the deflector seal 17412 when the water reservoir 17110 is positioned within the chamber 17130. For example, one end of the dividing gasket 17420 may contact an end of the deflector seal 17412. Another end of the deflector seal 17412 may contact a portion of the pathway seal 17424. This may form a sealed area that excludes the tub inlet 17160.

As pressurized air exits the chamber outlet 17264, the deflector seal 17412 may direct the airflow 17400 toward the sealing surface 17416 and inferior to the tub inlet 17160. The dividing gasket 17420 and the pathway seal 17424 extend along either side in order to limit the flow of pressurized gas toward either lateral side 17408, and instead directs the flow toward the surface 17148 and the heating element 17240. A seal may not extend between the pathway seal 17424 and the dividing gasket 17420 in order to allow the flow of pressurized air to travel inferior to the skirt 17428. However, the dividing gasket 17420 may extend below the skirt 17428 in order to limit backflow.

After traveling below the skirt 17424, the airflow 17400 is directed around at least a portion of the water reservoir 17110 by the pathway seal 17424. For example, the pathway seal 17424 forms a superior end of the pathway, and the walls of the water reservoir 17110, the walls of the chamber 17130, and the chamber base 17176 form sides and an inferior end of the pathway, thus constraining the fluid flow within the boundary. The dividing gasket 17420 may also limit the flow of air to one direction (e.g., counter clockwise as illustrated in FIG. 6F) around the perimeter of the water reservoir 17110.

The pathway seal 17424 may direct the flow of airflow 17400 away from the sealing surface 17416 and along the lateral side 17408. Proximate to an opening of the groove 17404, a portion of the pathway seal 17424 may limit further travel along the lateral side 17408, and instead may direct the flow into the groove 17404.

In some forms, the interior of the groove 17404 may include a sealing member (e.g., sealing between the surface 17148 adjacent to the groove 17404 and the heating element 17140). In other forms, a sealing member may not be formed on the interior of the groove 17404 (e.g., contact between the surface 17148 and the heating element 17240 may be sufficient to form a seal along the length of the groove 17404.

The fluid may flow through the groove 17404 and exit along the opposite lateral side 17408. A similar seal arrangement may be present, which directs the flow of pressurized air along the lateral side 17408 back toward the sealing surface 17416.

The flow of pressurized air may return toward the sealing surface 17416, where the dividing gasket 17420 and the pathway seal 17424 may direct the airflow 17400 toward the tub inlet 17460. The tub inlet 17160 may be within the sealing perimeter of the pathway seal 17424, but outside the perimeter of the deflector seal 17412. This allows the airflow 17400 that has travelled around the pathway to enter the tub inlet 17160 without flowing back toward the chamber outlet 17264 or back around the pathway (e.g., completing another lap around the perimeter of the water reservoir 17110).

In other examples, there may be an opening (e.g., a one way valve) that may allow a certain amount of air to be diverted from the tub opening 17160 and reintroduced into the pathway with the air exiting the chamber outlet 17264.

The airflow 17400 may travel along the sealing surface 17416 and enter the water reservoir 17110 through the tub inlet 17160 in order to be humidified as described above. The humidified airflow may then exit the tub through the tub outlet 17172 and directly into the chamber inlet 17192. For example, the two openings (i.e., the tub outlet 17172 and the chamber inlet 17192) may seal against one other so that the flow path is substantially continuous exiting the tub outlet 17172 and entering the chamber inlet 17192.

As described above, the pressurized gas may become pre-heated as it flows within the chamber 17130. Heat released from the heating element 17240 may warm the air as it travels around the pathway. Longer pathways may allow for a greater change in temperature. The length of the pathway may be affected by how far around the perimeter of the water reservoir 17110 that the pathway extends. Additionally, the length of the groove 17404 may affect the amount of heat transfer. For example, a longer groove 17404 (e.g., extending along a curved path) may allow for more heat transfer because the airflow 17400 is within the chamber 17130 and in close proximity to the heating element 17240 for a longer period of time (e.g., as compared to a linear groove 17404). However, as described below, the total change in temperature as a result of pre-heating may be optimized so that the final temperature of the air delivered to the patient is a pre-determined value (e.g., about 43-44° C.). Therefore, it may not be beneficial to create the longest possible pathway for pre-heating air if the final temperature delivered to the patient will exceed the desired value.

As shown in FIG. 5F, the chamber base 7176 supports a lid closure member 7204 that is movable between an open position and a closed position. The lid closure member 7204 comprises latches 7206 and a lid opening member 7208, which is provided at an end of the lid closure member 7204.

In certain forms, the lid closure member 7204 may be biased (e.g., spring-biased) toward the closed position. The patient may provide a force to the lid opening member 7208 in order to overcome the spring-bias, and move the lid closure member 7204 toward the open position.

In some forms, the chamber base 7176 may include hinge portions 7260. The hinge portions 7260 may be disposed opposite to the lid closure member 7204. For example, the hinge portions 7260 may be disposed on one chamber side wall 7188, and the lid closure member 7204 may be disposed on an opposite chamber side wall 7188, which faces the chamber side wall 7188 of the hinge portions 7260.

As shown in FIG. 5G, some forms of the chamber base 7176 humidifier chamber outlet 7264 to allow the humidified flow to be delivered to a delivery hose, tube, or conduit that is configured to be connected to the humidifier to deliver the humidified flow to a patient. The humidifier chamber outlet 7264 may be aligned with the outlet 7172, so humidified gas exiting the water reservoir 7110 may be directed toward the humidifier chamber outlet 7264. A sealing member (not shown) may be disposed proximate to the humidifier chamber outlet 7264 in order to create a sealing engagement between the humidifier chamber outlet 7264 and the outlet 7172 of the water reservoir 7110.

### 5.6.2.4.2 Chamber Lid

As shown in FIGS. 5D-5H, some forms of the chamber 7130 include a chamber lid 7268, which may be coupled to the chamber base 7176, and may at least partially cover an internal volume of the chamber base 7176.

In some forms, the chamber lid 7268 is movably coupled to the chamber base 7176, and is movable between an open position (e.g., where the internal volume of the chamber base 7176 is at least partially exposed) and a closed position (e.g., where the internal volume of the chamber base 7176 is covered).

In certain forms, the chamber lid 7268 may be pivotably coupled to the chamber base 7176, and may pivot between the open position and the closed position. The chamber lid 7268 may include a hinge portion 7272 that is hinged to the hinge portions 7260 of the chamber base 7176 (see e.g., FIG. 5F).

As shown in FIG. 5F, some forms of the chamber lid 7268 include catches 7276 that are configured to be engaged by the latches 44 to maintain the lid in the closed position. While in the closed position, providing a force to the lid opening member 7208 may move the latches 7206 out of engagement with the catches 7276 so that the chamber lid 7268 may pivot toward the open position.

In some forms, the chamber lid 7268 includes a window 7280 constructed from a transparent and/or translucent material. The window 7280 may allow the patient to visually inspect the interior of the chamber 7130 while the chamber lid 7268 is in the closed position. For example, the window 7280 may allow the patient to visually inspect the water reservoir 7110 received within the chamber base 7176.

As shown in FIGS. 5F and 5H, a lid seal 7284 may be coupled to some forms of the chamber lid 7268. The lid seal 7284 may engage the chamber base 7176 when the chamber lid 7268 is in the closed position. The lid seal 7284 may assist in maintaining a pressurized environment within the chamber 7130.

In some forms, the lid seal 7284 may include protrusions 7288 that may engage the water reservoir 7110 when the chamber lid 7268 is in the closed position. For example, the protrusions 7288 may engage the tub lid 7144.

In certain forms, the protrusions 7288 may be wedge-shaped, and may engage an inclined surface of the tub base 7140. The wedge-shaped protrusions 7288 may apply a force to the water reservoir 7110, which may push the water reservoir 7110 in a direction (e.g. laterally) toward the humidifier chamber outlet 7264 of the chamber 7130 to assist in forming a seal between the outlet 7172 and the humidifier chamber outlet 7264.

In some forms, the lid seal 7284 may include a domed portion 7292 that may engage the water reservoir 7110 when the chamber lid 7268 is in the closed position. For example, the protrusions 7288 may engage the tub lid 7144.

In certain forms, the domed portion 7292 may push the water reservoir 7110 in a direction (e.g., vertically downward) toward the heating element 7240 in order to firmly engage the tub bottom surface 7148 with the heating element 7240, and increase the efficiency of the conductive heat transfer.

In some forms, the lid seal 7284 circular seal section or sealing ring 7296 that may engage the water reservoir 7110 when the chamber lid 7268 is in the closed position. For example, the protrusions 7288 may engage the tub lid 7144.

The sealing ring 7296 may contact the tub lid 7144 proximate to the tub emptying aperture 7164. The sealing ring 7296 may limit pressurized air from entering or exiting the water reservoir 7110 through the tub emptying aperture 7164 while the chamber lid 7268 is in the closed position.

In some forms, the lid seal 7284 may include an inner sealing rim 7300 that is provided around an aperture 7304 of the lid seal 7284. The inner sealing rim 7300 may seal around the window 7280 of the chamber lid 7268. The aperture 7304 may be larger than the window 7280 in order to not obstruct the patient's view through the window.

As shown in FIG. 5H, some forms of the lid seal 7284 may be removable from the chamber lid 7268. A patient may remove the lid seal 7284 for cleaning. The lid seal 7284 may be re-coupled to the chamber lid 7268, or a new lid seal 7284 may be used.

As shown in FIGS. 5K and 5L, the lid seal 7284 may include a sealing rim 7285, which may be disposed around the outer perimeter of the lid seal 7284. The sealing rim 7285 may contact the chamber base 7176 when the chamber lid 7268 is in the closed position. Although in other examples, the sealing rim 7285 may be disposed on the chamber base 7176.

In some forms, the sealing rim 7285 may seal around a perimeter of the chamber base 7176 (e.g., along an outer perimeter of an opening to the chamber base 7176). The entire chamber 7130 may be pressurized when the chamber lid 7268 is closed (and when the flow of pressurized breathable gas is supplied).

In certain forms, the sealing rim 7285 may include a substantially cantilevered structure. The sealing rim 7285 may be constructed from a compliant material, and may at least partially compress against the chamber base 7176 when the chamber lid 7268 is moved into the closed position.

In certain forms, the sealing rim 7285 may direct the pressurized breathable gas within the chamber 7130 away from the interface between the chamber base 7176 and the chamber lid 7268, so that limited or no pressurized gas is able to escape the chamber 7130 (e.g., therefore helping to increase the efficiency of the humidifier 7000).

In some forms, the chamber lid 7268 may pressurize the volume of the chamber base 7176 (e.g., via the sealing rim 7285). The RPT device 4000 may be outside of this pressurized volume. In other words, the RPT device 4000 may not be disposed radially within a volume that the lid seal 7284 seals against.

### 5.6.2.4.3 Pre-Heating Airflow Path

As shown in FIGS. 5I-5T, the chamber 7130 may include an airflow pathway 7308. The pressurized breathable gas may flow through the airflow pathway 7308 after entering the chamber 7130 (e.g., through the chamber opening 7192—see e.g., FIGS. 5O and 5P), and may exit the airflow pathway 7308 proximate to the water reservoir 7110 (e.g., proximate to the outlet 7160—see e.g., FIG. 5I).

FIG. 5I illustrates a cross-sectional view of one example of the humidifier in an assembled, in use position. For example, the chamber lid 7268 is in a closed position where the lid seal 7284 contacts the chamber base 7176 in order to seal the interior of the chamber 7130.

In this in use position, the chamber bottom surface 7180 may contact a support surface (e.g., a table—not shown) that assists in maintaining the humidifier 7000 in the illustrated orientation. The window 7280 is therefore oriented opposite to the support surface so that a patient may view into the chamber 7130.

As illustrated in FIG. 5I, some forms of the airflow pathway 7308 are formed within the volume of the chamber base 7176 between the chamber 7130 and the water reservoir 7110. For example, the airflow pathway 7308 may be formed between a side wall 7152 of the water reservoir 7110 and a chamber side wall 7188 of the chamber 7130. As described previously, the water reservoir 7110 may be smaller than the chamber 7130 so that the side walls 7152 are spaced apart from the chamber side walls 7188, thus forming the airflow pathway 7308.

In the cross-section illustration of FIG. 5I, the airflow pathway 7308 may have a substantially rectangular shape, although other shapes (e.g., circular, elliptical, triangular, etc.) may be used. The illustrated airflow pathway 7308 includes a larger height (e.g., measured in a direction between the chamber bottom surface 7180 and the chamber lid 7268) than a width (e.g., measured between the chamber side wall 7188 and the side wall 7152), although other configurations (e.g., a width larger than the height) may be used.

FIG. 5I may also illustrate a substantially constant cross section of the airflow pathway 7308. The constant cross section may be formed as a result of the side wall 7152 and the chamber side wall 7188 oriented substantially parallel with respect to one another when the water reservoir 7110 is positioned within the chamber 7130. The parallel orientation of the side walls 7152, 7188 may limit the tapering of the airflow pathway 7308 in order to maintain a substantially constant airflow velocity through the airflow pathway 7308. In other words, the velocity of the pressurized breathable gas entering the chamber 7130 may be adjusted by the RPT device 4000, but the velocity may be substantially constant traveling through the airflow pathway 7308.

This in use position may allow pressurized airflow 7400 to pass through the airflow pathway 7308 as a result of the lid seal 7284 being in contact with the chamber base 7176.

In other forms (not shown), the airflow pathway 7308 may be formed between the tub base 7140 and the heating element 7240. For example, the tub base 7140 may be at least partially raised from the heating element 7240 in order to create an airflow pathway 7308 beneath the tub base 7140. The pressurized air may flow through this airflow pathway 7308 under the tub base 7140 and over top of the heating element 7240 (e.g., flowing directly against the heating element 7240) in order to be heated. This airflow pathway 7308 may be shorter than the airflow pathway 7308 described above that flows around at least part of the perimeter of the tub base 7140.

In other forms (not shown), the side wall 7152 and the chamber side wall 7188 may not be parallel but may still form an airflow pathway 7308 with a constant cross section. For example, the side wall 7152 and the chamber side wall 7188 may both be curved or rounded (e.g., formed with a circular or elliptical shape). However, the water reservoir 7110 may be concentric with the chamber 7130 and formed with the same shape (e.g., both circles) so that the airflow pathway 7308 has a constant cross-section despite non-parallel walls.

In other words, even without parallel walls 7152, 7188, the airflow pathway 7308 may include a constant cross-section if the distance between the walls 7152, 7188 remains constant around the perimeter of the airflow pathway 7308.

In still other forms (not shown), the distance between the side wall 7152 and the chamber side wall 7188 may change along the length of the walls 7152, 7188. In other words, the cross section may not be constant, and may instead be variable. The variable cross section may create different flow velocities (e.g., as opposed to a substantially constant flow velocity when using a constant cross section) around different sections of the walls 7152, 7188. For example, larger widths between the walls 7152, 7188 may result in lower flow velocities.

FIG. 5J illustrates a detail view of the humidifier of FIG. 5I in order to better illustrate a portion of the airflow pathway 7308 described above. As previously noted, the lateral sides of the airflow pathway 7308 may be formed between a side wall 7152 of the water reservoir 7110 and a chamber side wall 7188 of the chamber 7130.

As illustrated in FIG. 5J, some forms of the humidifier include an upper seal 7312 that may extend between the side wall 7152 and the chamber side wall 7188. The upper seal 7312 may contact both the side wall 7152 and the chamber side wall 7188 in a sealing engagement in order to limit or prevent airflow 7400 in the superior direction (e.g., toward the chamber lid 7268 of FIG. 5I when the humidifier is in the in use position). Thus, the upper seal 7312 may form the superior end of the airflow pathway 7308. The upper seal 7312 may be formed from a flexible and/or resilient material that permits bending and/or flexing.

In other forms (not shown), the upper seal 7312 may be disposed proximate to a superior portion of the water reservoir 7110 (e.g., the upper seal 7312 may be formed on the tub lid 7144 or the chamber lid 7268). Positioning the upper seal 7312 higher may create a larger pathway 7308 (e.g., taller in the superior direction). The different position of the upper seal 7312 may also affect the assembly (e.g., the direction of assembly) of the chamber base 7176 and the water reservoir 7110. The more superior position of the upper seal 7312 may also allow for a different seal styles (e.g., other than a face seal).

In certain forms, the upper seal 7312 may be fixed to the chamber side wall 7188, and may extend toward a center of the chamber base 7176. For example, FIG. 5J illustrates that the upper seal 7312 may be coupled to the chamber side wall 7188 in a cantilevered arrangement. However, other examples (not shown) may include the upper seal 7312 fixed to the side wall 7152 and may extend toward an edge of the chamber base 7176.

With continued reference to FIG. 5J, certain forms of the humidifier 7000 may include the upper seal 7312 that is longer (e.g., the distance from a fixed end to a free end) than the distance between the side wall 7152 and the chamber side wall 7188 (e.g., the width of the airflow pathway 7308). When the water reservoir 7110 is inserted into the chamber base 7176, the tub base 7140 may first contact the upper seal 7312, causing it to bend. As shown, the upper seal 7312 may bend downwardly (e.g., in an inferior direction, in use) and toward the chamber side wall 7188. The upper seal 7312 may form a face seal with the side wall 7152 (e.g., the side wall 7152 is substantially perpendicular to the direction that the upper seal 7312 extends from the chamber side wall 7188).

In certain forms, the engaged orientation of the upper seal 7312 may form a face seal. In other words, the sealing surface may be substantially perpendicular to the direction of engagement. For example, the illustrated upper seal 7312 may extend from the chamber side wall 7188 to the side wall 7152 (illustrated along a horizontal plane), and may contact the side wall 7152 in a perpendicular direction (illustrated along a vertical plane).

As shown in FIG. 5J, the upper seal 7312 may be held in its position by the water reservoir 7110 (e.g., the side wall 7152), so that that the upper seal 7312 may remain in sealing engagement with the side wall 7152 while the water reservoir 7110 is disposed within the chamber 7130.

In some examples, the upper seal 7312 may act as a pressure assisted seal during operation of the humidifier 7000. The pressurized breathable gas traveling through the airflow pathway 7308 may provide a force against the upper seal 7312 in the superior direction. The upper seal 7312 may be pushed into firm contact with the side wall 7152 in order to prevent or limit air leak in the superior direction.

Once the pressurized breathable gas has been entered the airflow pathway 7308, the upper seal 7312 acts to direct the airflow 7400 around the airflow pathway 7308 (e.g., by preventing or limiting the airflow 7400 in the superior direction). In some forms, the upper seal 7312 extends around the entire length of the airflow pathway 7308. This allows for sealing between the chamber base 7176 and the tub base 7140 along the entire length of the airflow pathway 7308 (e.g., so that pressurized air is limited from leaking along the length).

With continued reference to FIG. 5J, some forms of the humidifier 7000 may include a lower seal 7316 that may extend between the side wall 7152 and the chamber side wall 7188. The lower seal 7316 may contact both the side wall 7152 and the chamber side wall 7188 in a sealing engagement in order to limit or prevent airflow 7400 in the inferior direction. Thus, the lower seal 7316 may form the inferior end of the airflow pathway 7308.

In some forms, the upper seal 7312 may be spaced apart from the lower seal 7316 so that the seals 7312, 7316 are not in contact with one another. The distance between the upper and lower seals 7312, 7316 may be the height of the airflow pathway 7308. In some examples, the distance between the upper and lower seals 7312, 7316 may remain substantially constant along the length of the airflow pathway 7308. This may assist in providing the constant cross section described above.

As shown in FIG. 5J, certain forms of the lower seal 7316 may be fixed to the chamber side wall 7188, and may extend toward a center of the chamber base 7176. For example, the lower seal 7316 may be coupled to the chamber side wall 7188 in a cantilevered arrangement. However, other forms of the lower seal 7316 may be fixed to the side wall 7152 and extend toward an outer periphery of the chamber base 7176.

In certain forms, the lower seal 7316 may be longer (e.g., the distance from a fixed end to a free end) than the distance between the side wall 7152 and the chamber side wall 7188 (e.g., the width of the airflow pathway 7308). When the water reservoir 7110 is inserted into the chamber base 7176, the tub base 7140 may contact the lower seal 7316, causing it to bend. As shown, the lower seal 7316 may bend downwardly (e.g., in an inferior direction, in use) and back toward the chamber side wall 7188. For example, the free end of the cantilevered lower seal 7316 may bend back toward the chamber side wall 7188 as a result of contact with the tub base 7140 and the side wall 7152.

The lower seal 7316 may be held in its position by the water reservoir 7110 (e.g., the side wall 7152), so that that the lower seal 7316 may remain in sealing engagement with the side wall 7152 while the water reservoir 7110 is disposed within the chamber 7130. In this position, the lower seal 7316 may contact the heating element 7240.

With continued reference to FIG. 5J, one form of the humidifier 7000 may include the tub base 7140 with an inclined wall 7320 that contacts the lower seal 7316 when the water reservoir 7110 is inserted into the chamber 7130. The inclined wall 7320 may press the lower seal 7316 toward the heating element 7240 as the chamber lid 7268 pushes the water reservoir 7110 toward the heating element 7240. The lower seal 7316 may be resilient, and may biased toward the superior direction, thereby forming a firm engagement with the tub base 7140. The lower seal 7316 may form a face seal with the side wall 7152 (e.g., the lower seal 7316 may similarly engage the inclined wall 7320 as the upper seal 7312 engages the side wall 7152).

Once the pressurized breathable gas has been entered the airflow pathway 7308, the lower seal 7316 may act to direct the airflow 7400 around the airflow pathway 7308 (e.g., by preventing or limiting the airflow 7400 in the inferior direction). In some forms, the lower seal 7316 extends around the entire length of the airflow pathway 7308. This allows for sealing between the chamber base 7176 and the tub base 7140 along the entire length of the airflow pathway 7308 (e.g., so that pressurized air is limited from leaking along the length).

In other forms, the humidifier 7000 may not include the lower seal 7316, and may only seal the airflow pathway 7308 using the upper seal 7312. The inferior portion of the airflow pathway 7308 may be formed by the heating element 7240.

As shown in FIGS. 5I and 5J, the upper and lower seals 7312, 7316 may be positioned so that the airflow pathway 7308 is at least partially in the bottom half of the chamber 7130. This may position the airflow pathway 7308, and therefore the pressurized air flowing through the pathway 7308, closer to the heating element 7240, which may promote heat transfer. However, it is noted that because warm air rises, circulating the flow of pressurized air in an upper portion of the chamber 7130 (e.g., by positioning the upper and/or lower seal 7312, 7316 further from the heating element 7240) may still provide effective heat transfer.

FIGS. 5K and 5L illustrate a side cross-sectional view of the humidifier 7000, showing the engagement between the chamber lid 7268 and the chamber base 7176. As part of the engagement, a sealing rim 7285 may contact the chamber base 7176 when the chamber lid 7268 is in the closed position. The lid seal 7284 may include a sealing rim 7285, which may be disposed around the outer perimeter of the lid seal 7284. Although in other examples, the sealing rim 7285 may be disposed on the chamber base 7176, and may contact the chamber lid 7268 when the chamber lid 7268 is in the closed position.

With continued reference to FIGS. 5K and 5L, the chamber base 7176 may include an entry chamber 7328, which may be part of the airflow pathway 7308 described above. The humidifier 7000 may also include an entry opening 7324 described in more detail below. The entry chamber 7328 and the entry opening 7324 may be part of the airflow pathway 7308 described above. As illustrated example, the entry chamber 7328 and the entry opening 7324 may be divided from at least a portion of the airflow pathway 7308. As described in more detail below, this may assist in forcing the pressurized air to travel around the tub base 7140 prior to entering the tube base 7140.

As shown in FIG. 5L, some forms of the entry opening 7324 may include a substantially L-shaped opening when viewed in cross section. In other words, pressurized breathable gas flowing through the entry opening 7324 may flow in both the superior direction and the lateral direction. As described above, the entry seal 7322 may have an elongated shape, so that the entry opening 7324 forms a passageway.

In some forms, the entry opening 7324 may direct the pressurized flow of breathable gas from the airflow pathway 7308 toward the channel 7168 on the tub lid 7144. For example, the substantially horizontal portion of the entry opening 7324 may direct the pressurized flow of breathable gas toward a center of the chamber 7130 (e.g., away from the chamber side walls 7188). The substantially horizontal portion of the entry opening 7324 may be generally aligned with the channel 7168 so that airflow 7400 leaving the entry opening 7324 may be conveyed directly into the channel 7168 and the outlet 7160. In some examples, the horizontal portion of the entry opening 7324 may extend at least partially over the water reservoir 7110 in order to reach the channel 7168.

FIG. 5M illustrates a cross-sectional view of the humidifier 7000, viewed from the front of the humidifier 7000. From this view, the entry chamber 7328 and the entry opening 7324 may be observed.

In some forms, the entry chamber 7328 of the chamber base 7176 may form an end of the airflow pathway 7308 (e.g., the pressurized flow of breathable gas may flow from the chamber opening 7192 to the entry chamber 7328). In the illustrated example, the chamber opening 7192 and the entry chamber 7328 are approximately 360° apart, although the entry chamber 7328 may be disposed any angular distance from the chamber opening 7192.

In some forms, the entry opening 7324 may be aligned with the entry chamber 7328, so that the pressurized air may flow into the entry opening 7324 after reaching the entry opening 7324. As shown in FIG. 5M, the entry opening 7324 and the entry chamber 7328 may be substantially vertically aligned.

The entry seal 7322 may limit the pressurized air from exiting the entry chamber 7328 (e.g., in a superior direction), and not traveling through the entry opening 7324 (e.g., and instead traveling elsewhere through the chamber 7130).

As shown in the cross-sectional view of FIG. 5N, the airflow pathway 7308 may extend at least partially around the perimeter of the chamber 7130. In other words, the chamber opening 7192 and the outlet 7172 may be angularly spaced from one another. This may allow a conduit for connecting to the patient interface 3000 to be spaced apart from the chamber opening 7192.

In some forms, the airflow pathway 7308 may extend so that the chamber opening 7192 and the entry opening 7324 are at least 90° apart from one another around the chamber 7130 (e.g., approximately one quarter of the perimeter of the chamber base 7176). In some forms, the airflow pathway 7308 may extend around the chamber 7130 so that the chamber opening 7192 and the entry opening 7324 are at least 180° apart (e.g., approximately one half of the perimeter of the chamber base 7176). In some forms, the airflow pathway 7308 may extend so that the chamber opening 7192 and the entry opening 7324 are at least 270° around the chamber 7130 (e.g., approximately three quarters of the perimeter of the chamber base 7176). In some forms, the airflow pathway 7308 may extend so that the chamber opening 7192 and the entry opening 7324 are approximately 360° apart from one another so that the airflow pathway 7308 extends around the chamber 7130 (e.g., approximately the entire of the perimeter of the chamber base 7176).

In other words, if the airflow pathway 7308 extends approximately the entire way around the chamber 7130, the chamber opening 7192 and the entry opening 7324 may be disposed at approximately the same angular position of the chamber 7130, although the flow of pressurized breathable gas has to travel along substantially the entire perimeter of the chamber 7130 in order to reach the outlet 7160 from the chamber opening 7192.

As described above, the outlet 7172 may be spaced apart from the chamber opening 7192. As shown in FIG. 5N, the airflow pathway 7308 may extend past the outlet 7172 without providing communication with the outlet 7172. For example, the airflow pathway 7308 may be below the opening for the outlet 7172. This limits un-humidified airflow 7400 that exits through the outlet 7172.

As shown in FIGS. 5N-5P, the humidifier 7000 may include an entry seal 7322 with a humidifier tub entry opening 7324, which may provide an opening for the pressurized breathable gas to reach the outlet 7160.

In some forms, the entry seal 7322 may extend around an entire perimeter of the chamber base 7176, in order to provide sealing to airflow 7400 throughout the chamber base 7176 (e.g., along the substantially the entire length of the airflow pathway 7308).

In some forms, the entry seal 7322 may include an elongated shape, and may extend away from its sealing surface on the chamber base 7176. For example, the entry seal 7322 may extend in the superior direction away from the chamber base 7176, while the humidifier 7000 is in use.

In some forms, the entry seal 7322 may be coupled to the chamber base 7176, and may extend toward the chamber lid 7268. The chamber lid 7268 may contact a superior portion of the entry seal 7322 when the chamber lid 7268 is in the closed position. The entry seal 7322 may also be formed from a compliant material (e.g., silicone) in order to allow the chamber lid 7268 to close while in contact with the entry seal 7322).

As shown in FIGS. 5O and 5P, some forms of the humidifier 7000 may include a perimeter of the entry opening 7324 that is fully formed by the entry seal 7322. In other words, the entry seal 7322 may seal against a surface of the chamber base 7176 along the entire perimeter of the entry opening 7324, but not across the entry opening 7324. This may direct the flow of air through the entry opening 7324, and not around the outside of the entry opening 7324.

In certain forms, the entry opening 7324 may have a substantially rectangular shape. As shown in FIG. 5P, the entry opening 7324 may be slightly curved, but includes parallel sides, and sides oriented perpendicularly with respect to one another. In other forms, the entry opening 7324 may have a different shape (e.g., circular, elliptical, triangular, etc.).

As shown in FIG. 5Q, the entry seal 7322 is disposed more superior than the airflow pathway 7308, and may cover the entire airflow pathway 7308. The entry seal 7322 may be disposed more superior than the upper seal 7312 so that the pressurized breathable gas does not flow between the upper seal 7312 and the entry seal 7322.

In some forms, the chamber base 7176 includes an entry chamber 7328, which may form an end of the airflow pathway 7308 (e.g., the pressurized flow of breathable gas may flow from the chamber opening 7192 to the entry chamber 7328). In the illustrated example, the chamber opening 7192 and the entry chamber 7328 are approximately 360° apart, although the entry chamber 7328 may be disposed any angular distance from the chamber opening 7192.

In some forms, the entry opening 7324 may be aligned with the entry chamber 7328, so that the pressurized air may flow into the entry opening 7324 after reaching the entry opening 7324. The entry seal 7322 may limit the pressurized air from exiting the entry chamber 7328 (e.g., in a superior direction), and not traveling through the entry opening 7324 (e.g., and instead traveling elsewhere through the chamber 7130).

As shown in FIGS. 5R and 5S, a pathway seal 7332 is disposed between the entry chamber 7328 and the start of the airflow pathway 7308 (e.g., the portion directly communicating with the chamber opening 7192).

In some forms, pathway seal 7332 may extend between the side wall 7152 and the chamber side wall 7188. The pathway seal 7332 may contact both the side wall 7152 and the chamber side wall 7188 in a sealing engagement in order to limit or prevent airflow 7400 in the superior direction.

In certain forms, the pathway seal 7332 may be fixed to the chamber side wall 7188, and may extend toward a center of the chamber base 7176. For example, the pathway seal 7332 may be coupled to the chamber side wall 7188 in a cantilevered arrangement.

In certain forms, the pathway seal 7332 may be longer (e.g., the distance from a fixed end to a free end) than the distance between the side wall 7152 and the chamber side wall 7188 (e.g., the width of the airflow pathway 7308). When the water reservoir 7110 is inserted into the chamber base 7176, the tub base 7140 may contact the pathway seal 7332, causing it to bend. As shown, the pathway seal 7332 may bend laterally (e.g., in a leftward direction, as shown in FIG. 5S). The pathway seal 7332 may form a face seal with the side wall 7152 (e.g., the side wall 7152 is substantially perpendicular to the direction that the upper seal 7312 extends from the chamber side wall 7188).

In certain forms, the pathway seal 7332 may be oriented differently than the upper and lower seals 7312, 7316. For example, the pathway seal 7332 may be rotated approximately 90° with respect to the upper and lower seals 7312, 7316. The pathway seal 7332 may seal against a height of the side wall 7152, and not along a length of the side wall 7152.

The pathway seal 7332 may be held in its position by the water reservoir 7110 (e.g., the side wall 7152), so that that the pathway seal 7332 may remain in sealing engagement with the side wall 7152 while the water reservoir 7110 is disposed within the chamber 7130.

In some examples, the pathway seal 7332 may act as a pressure assisted seal during operation of the humidifier 7000. The pressurized breathable gas entering the airflow pathway 7308 may provide a force against the pathway seal 7332 in the lateral direction (e.g., toward the right as viewed in FIG. 5S). The pathway seal 7332 may be pushed into firm contact with the side wall 7152 in order to prevent or limit air leak in one lateral direction. In other words, the pathway seal 7332 helps to ensure that the pressurized flow of breathable gas flows along the entire length of the airflow pathway 7308 prior to reaching the entry chamber 7328 (e.g., as opposed to entering through the chamber opening 7192 and flowing immediately into the entry chamber 7328).

Once the pressurized breathable gas has been entered the airflow pathway 7308, the pathway seal 7332 acts to direct the airflow 7400 around the airflow pathway 7308 (e.g., by preventing or limiting the airflow 7400 directly into the entry chamber 7238). In some forms, the pathway seal extends the entire height of the airflow pathway 7308, and may contact the entry seal 7322 at a superior end (e.g., so that pressurized air is limited from leaking along the height).

### 5.6.2.4.4 Operation

In use, the humidifier 7000 is connected (i.e., either integrally or removably) to the RPT device 4000. As described above, the connection may be mechanical and/or electrical. When connected, the flow generator of the RPT device 4000 is spaced apart from the humidifier 7000 (e.g., and specifically the water reservoir 7110).

As shown at least in FIGS. 51 and 5K, the water reservoir 7110 may be positioned within the chamber base 7176 so that the bottom surface 7148 of the water reservoir 7110 is in contact with the heating element 7240.

In some forms, as the water reservoir 7110 is inserted, the side walls 7152 contact the seals (e.g., the upper seal 7312, the lower seal 7316, and/or the pathway seal 7332. The seals (e.g., constructed from a flexible material, like silicone) may bend as a result of contact with the water reservoir 7110, and may be biased toward their original position in order to seal against the surface of the water reservoir 7110.

The patient may operate the RPT device 4000 as described previously in order to generate a flow of pressurized breathable gas. When the humidifier 7000 is connected to the RPT device 4000 as shown in FIG. 5D (or in FIG. 6A with the alternative example), the flow of pressurized breathable gas is conveyed from the RPT device 4000 to the humidifier 7000. For example, the RPT device 4000 may include a flow generator outlet that is in communication with the chamber opening 7192. Air leaving the flow generator outlet may flow downstream and enter the volume of the chamber base 7176 (e.g., at least partially formed by the chamber walls 7188) through the chamber opening 7192.

The flow of pressurized breathable gas enters the humidifier 7000 through the chamber opening 7192. Specifically, the flow of pressurized breathable gas enters the volume of space that removably houses the water reservoir 7110 (e.g., at least partially formed by the chamber walls 7188). The sealing member 7196, illustrated in FIGS. 5N-5P, disposed within the chamber opening 7192 creates a seal (e.g., a face seal) between the humidifier 7000 and the RPT device 4000 in order to limit the flow of gas from leaking between the interfaces.

With continued reference to FIGS. 5N and 5O, some forms of the chamber base 7176 include a deflector wall 7336 in a facing relationship with the chamber opening 7192. In other words, the deflector wall 7336 may be oriented substantially perpendicularly with respect to the direction of airflow 7400 through the chamber opening 7192. A passage entryway 7340 may be disposed between the deflector wall 7336 and the chamber opening 7192, and may be oriented along a generally superior-inferior direction. The deflector wall 7336 may change the direction of airflow 7400 (e.g., from generally horizontal to generally vertical) so that the flow of pressurized breathable gas is directed into the airflow pathway 7308. The airflow pathway 7308 may therefore be located in a more inferior position than the chamber opening 7192

Once the flow of pressurized breathable gas enters the airflow pathway 7308, the pathway seal 7332 directs the flow along the length of the airflow pathway 7308. As described above, the pathway seal 7332 may limit airflow directly into the entry chamber 7328. Additionally, the upper and lower seals 7312, 7316 form the upper and lower boundaries, respectively, of the airflow pathway 7308. A combination of these seals 7312, 7316 limit the direction of travel along the airflow pathway 7308 to a single direction (e.g., clockwise as shown in FIG. 5Q).

As the pressurized breathable gas travels around the airflow pathway 7308, the heating element 7240 receives electrical power and produces heat. In addition to conductively heating the water reservoir 7110, heat produced by the heating element 7240 may convectively heat the airflow pathway 7308. In other words, the lower seal 7316 may not be constructed from a substantially insulating material, and heat from the heating element 7240 may travel into the airflow pathway 7308.

The heating element 7240 may produce heat in order to directly heat the water reservoir 7110, and therefore any liquid stored within (e.g., via conduction). Heat will also leave the heating element 7240 via convection. The flow of pressurized breathable gas is forced along the airflow pathway 7308, and is heated through forced convection. However, the process of heating the flow of pressurized air may be considered a passive process, since the heating element 7240 is not generating heat for the purpose of heating the flow of pressurized air. In other words, only an amount of heat needed to warm the liquid in the water reservoir 7110 may be generated by the heating element 7240. The heat used to warm the flow of pressurized air would otherwise be lost due to natural inefficiencies in any system (i.e., because convection occurs).

As the pressurized breathable gas flows along the airflow pathway 7308, heat from the heating element 7240 permeates the airflow pathway 7308 and warms the air.

Although the figures (e.g., FIG. 5J) illustrate the airflow pathway 7308 extending over the heating element 7240 (e.g., so that the heating element 7240 is vertically aligned with the airflow pathway 7308), the heating element 7240 may alternatively have a smaller surface area than the tub bottom surface 7148. In this alternative, the heating element 7240 may not be vertically aligned with the airflow pathway 7308. Heat produced by the heating element 7240 may still permeate into the airflow pathway 7308 in order to pre-heat the flow of pressurized air.

In some forms, the breathable gas may be continuously warmed along the length of the airflow pathway 7308. For example, the breathable gas may enter the airflow pathway 7308 (e.g., through the passage entryway 7340) at a first temperature.

In certain forms, the air entering the airflow pathway 7308 (e.g., the first temperature) may be approximately ambient temperature. For example, ambient temperature (e.g., between about 20° C. and about 22° C.) may enter the system as a whole and flow through the RPT device 4000.

In certain forms, the RPT device 4000 may generate heat and increase the temperature of the air so that the air leaving the RPT device 4000 and flowing into the humidifier 7000 is slightly above ambient temperature. For example, the RPT device 4000 may increase the temperature of the flow of air by about 0.1° C. to about 10° C. In some forms, the RPT device 4000 may increase the temperature of the flow of air by about 0.5° C. to about 7.5° C. In some forms, the RPT device 4000 may increase the temperature of the flow of air by about 0.75° C. to about 5° C. In some forms, the RPT device 4000 may increase the temperature of the flow of air by about 1° C. to about 2.5° C.

Convective heat transfer continues to occur as the breathable gas travels around the airflow pathway 7308 so that the temperature of the breathable gas increases above the first temperature. The breathable gas may not necessarily increase in temperature constantly (e.g., temperature may or may not be directly related to distance travelled along the airflow pathway 7308), although the temperature at any one point may be greater than the temperature at a previous point that is disposed closer (e.g., angularly closer) to the chamber opening 7192. Proximate to reaching the entry chamber 7328, the breathable gas has reached a second temperature, which is greater than the first temperature.

In some forms, the temperature (e.g., second temperature) of the airflow 7400 through the system may be at or near a maximum when the airflow 7400 enters the entry chamber 7328. The temperature of the flow of air may increase as described above as a result of the conductive heat transfer. In certain forms, the temperature may further increase while inside the water reservoir 7110 as a result of continued exposure to the heating element 7240. However, while within the water reservoir 7110 and/or after exiting the water reservoir 7110, the air temperature may decrease from the second temperature (e.g., because that portion of the airflow is no longer exposed to the heating element 7240).

In some forms, the second temperature may be between about 1° C. and about 200° C. greater than the first temperature. In some forms, the second temperature may be between about 10° C. and about 150° C. greater than the first temperature. In some forms, the second temperature may be between about 30° C. and about 100° C. greater than the first temperature. In some forms, the second temperature may be between about 50° C. and about 75° C. greater than the first temperature. In some forms, the second temperature may be between about 60° C. and about 70° C. greater than the first temperature. In some forms, the second temperature may be about 65° C. greater than the first temperature.

In certain forms, the temperature output to the patient (i.e., the temperature of the pressurized air within the plenum chamber 3200) may be between about 10° C. and about 100° C. In certain forms, the temperature output to the patient may be between about 25° C. and about 75° C. In certain forms, the temperature output to the patient may be between about 30° C. to about 50° C. In certain forms, the temperature output to the patient may be between about 43° C. and about 44° C. The humidifier system as a whole may be designed so that the temperature of the heating element 7240 and the length of the airflow pathway 7308 are an appropriate length to achieve the desired output temperature.

For example, the maximum temperature of the flow of pressurized air (e.g., influenced by the temperature of the heating element 7240, the length of the airflow pathway 7308, and/or any other factors) may be dependent on the overall design of the system. A longer air circuit 4170 may provide a greater amount of time for the airflow to cool after exiting the water reservoir 7110 (e.g., where it is no longer heated by the heating element 7240). This may allow for a greater maximum temperature of in the heating element 7240 and/or a longer airflow pathway 7308 so that the flow of pressurized air may be hotter, and therefore more humid after exiting the water reservoir 7110. Conversely, a shorter air circuit 4170 provides less time for the flow of pressurized air to cool to the patient output temperature, thus meaning that the heating element 7240 must have a lower maximum temperature and/or the airflow pathway 7308 must be shorter.

Other factors, like the surface area of the airflow pathway 7308, the temperature of the water within the water reservoir 7110, the surface temperature of the water reservoir 7110, the insulation of the chamber 7130, the humidity of the ambient environment, and/or any other considerations may also affect the maximum temperature of the flow of pressurized air.

Another consideration is that the humidified air may condensate if the temperature is increased too much above the ambient temperature. For example, significantly high temperatures, together with a long air circuit 4170, may cause substantial condensation along the air circuit 4170, which may decrease the efficiency of the humidifier 7000. To counter this, the air circuit 4170 in order to maintain a higher temperature of the humidified air traveling through the air circuit 4170. However, heating the air circuit 4170 means that the air entering the air circuit 4170 must be cooler (e.g., than if the air circuit 4170 was not heated) so that the air output by the air circuit 4170 (e.g., to the plenum chamber 3200) is the desired output temperature.

In light of these considerations, the humidifier 7000 is optimized so that the flow of pressurized air is increased enough to supply adequate humidify to the patient at an appropriate temperature, while reducing unnecessary inefficiencies in the humidifier 7000.

The humidifier 7000 increases the temperature of the ambient air by at least a few degrees Celsius (e.g., at least 1 to 2° C., 3 to 4° C., 5 to 10° C., etc.) in order to allow for increased air humidity after passing through the water reservoir 7110, and to cool to a desired temperature for inhalation (e.g., air temperature in a plenum chamber 3200).

As shown in FIG. 5L, the breathable gas at the second temperature is directed in an upwardly direction by the entry opening 7324, so that the heated air may enter the water reservoir 7110 (e.g., because the outlet 7160 is disposed more superior than the airflow pathway 7308).

In some forms, leaks may occur from the entry chamber 7328 back to the beginning of the airflow pathway 7308 (e.g., around the pathway seal 7332). Small amounts of air flowing in this direction may be allowable, although air flowing in the opposite direction (e.g., directly to the entry chamber 7328) may not be allowable.

The flow of breathable gas may then increase in humidity as a result of flowing through the water reservoir 7110. As described previously, the heating element 7240 warms the liquid in the water reservoir 7110 via conductive heating.

After increasing its humidity, the flow of pressurized breathable gas may exit the water reservoir 7110 through the outlet 7172 and the chamber outlet 7264, and may flow toward the patient (e.g., via a gas delivery tube).

As described above, using the airflow pathway 7308 in order to pre-heat the pressurized flow of breathable gas may assist in improving the efficiency of the humidifier 7000. Heat may be convectively released from the heating element 7240 regardless of the presence of the airflow pathway 7308. In other words, the heating element 7240 may include inherent inefficiencies. Pre-heating the flow of pressurized breathable gas allows the humidifier 7000 to reduce these inefficiencies by utilizing the already available convective heat. Since the flow of pressurized breathable gas is heated passively, the respiratory apparatus 7001 do not need to spend additional energy in order to pre-heat the flow of air.

Additionally, warmer air has a greater capacity to hold water vapor. For example, when the air temperature increases (e.g., from the first temperature to the second temperature) and the water vapor content stays the same (e.g., because the flow of gas has not yet entered the water reservoir 7110), the relative humidity of the air drops. In other words, the air at the higher temperature (e.g., the second temperature) is capable of holding more water than the air at the lower temperature (e.g., the first temperature), and thus the air at the higher temperature is holding a lower percentage of its maximum, as compared to air at the lower temperature.

Thus, pre-heating the airflow 7400 through the humidifier 7000 may benefit the patient because more water vapor may be inhaled by the patient using the patient interface 3000 connected to the humidifier 7000. This may help the patient limit experiencing a sore throat, or other side effects of using the patient interface 3000.

The positioning of the airflow pathway 7308 may also assist increasing the temperature change from the chamber opening 7192 to the entry chamber 7328 (i.e., the ΔT between the first and second temperatures). In the illustrated example, the airflow pathway 7308 is generally disposed in an inferior region of the chamber 7130. Additionally, the inferior end of the airflow pathway 7308 may be proximate to the heating element 7240. This may assist in limiting wasted heat that does not conductively heat the liquid in the water reservoir 7110 or convectively heat the pressurized gas.

In certain forms, the lower seal 7316 may be omitted. The heating element 7240 may therefore be the inferior end of the airflow pathway 7308. The flow of pressurized breathable gas may flow directly across the heating element 7240 in order to maximize the convective heating (although potentially at the expense of leaks in the airflow pathway 7308).

5.6.2.5 Water level Indicator

The humidifier reservoir 5110 may comprise a water level indicator 5150 as shown in FIG. 5A-5B. In some forms, the water level indicator 5150 may provide one or more indications to a user such as the patient 1000 or a care giver regarding a quantity of the volume of water in the humidifier reservoir 5110. The one or more indications provided by the water level indicator 5150 may include an indication of a maximum, predetermined volume of water, any portions thereof, such as 25%, 50% or 75% or volumes such as 200 ml, 300 ml or 400 ml.

### 5.6.2.6 Humidifier Transducer(s)

The humidifier 5000 may comprise one or more humidifier transducers (sensors) 5210 instead of, or in addition to, transducers 4270 described above. Humidifier transducers 5210 may include one or more of an air pressure sensor 5212, an air flow rate transducer 5214, a temperature sensor 5216, or a humidity sensor 5218 as shown in FIG. 5C. A humidifier transducer 5210 may produce one or more output signals which may be communicated to a controller such as the central controller and/or the humidifier controller 5250. In some forms, a humidifier transducer may be located externally to the humidifier 5000 (such as in the air circuit 4170) while communicating the output signal to the controller.

### 5.6.2.6.1 Pressure Transducer

One or more pressure transducers 5212 may be provided to the humidifier 5000 in addition to, or instead of, a pressure sensor provided in the RPT device 4000.

5.6.2.6.2 Flow rate Transducer

One or more flow rate transducers 5214 may be provided to the humidifier 5000 in addition to, or instead of, a flow rate sensor provided in the RPT device 4000.

### 5.6.2.6.3 Temperature Transducer

The humidifier 5000 may comprise one or more temperature transducers 5216. The one or more temperature transducers 5216 may be configured to measure one or more temperatures such as of the heating element 5240 and/or of the flow of air downstream of the humidifier outlet 5004. In some forms, the humidifier 5000 may further comprise a temperature sensor 5216 to detect the temperature of the ambient air.

### 5.6.2.6.4 Humidity Transducer

In one form, the humidifier 5000 may comprise one or more humidity sensors 5218 to detect a humidity of a gas, such as the ambient air. The humidity sensor 5218 may be placed towards the humidifier outlet 5004 in some forms to measure a humidity of the gas delivered from the humidifier 5000. The humidity sensor may be an absolute humidity sensor or a relative humidity sensor.

### 5.6.2.7 Humidifier Controller

According to one arrangement of the present technology, a humidifier 5000 may comprise a humidifier controller 5250 as shown in FIG. 5C. In one form, the humidifier controller 5250 may be a part of the central controller. In another form, the humidifier controller 5250 may be a separate controller, which may be in communication with the central controller.

In one form, the humidifier controller 5250 may receive as inputs measures of properties (such as temperature, humidity, pressure and/or flow rate), for example of the flow of air, the water in the reservoir 5110 and/or the humidifier 5000. The humidifier controller 5250 may also be configured to execute or implement humidifier algorithms and/or deliver one or more output signals.

As shown in FIG. 5C, the humidifier controller 5250 may comprise one or more controllers, such as a central humidifier controller 5251, a heated air circuit controller 5254 configured to control the temperature of a heated air circuit 4171 and/or a heating element controller 5252 configured to control the temperature of a heating element 5240.

### 5.7 Breathing Waveforms

FIG. 7 shows a model typical breath waveform of a person while sleeping. The horizontal axis is time, and the vertical axis is respiratory flow rate. While the parameter values may vary, a typical breath may have the following approximate values: tidal volume Vt 0.5 L, inhalation time Ti 1.6 s, peak inspiratory flow rate Qpeak 0.4 L/s, exhalation time Te 2.4 s, peak expiratory flow rate Qpeak −0.5 L/s. The total duration of the breath, Ttot, is about 4 s. The person typically breathes at a rate of about 15 breaths per minute (BPM), with Ventilation Vent about 7.5 L/min. A typical duty cycle, the ratio of Ti to Ttot, is about 40%.

### 5.8 Glossary

For the purposes of the present technology disclosure, in certain forms of the present technology, one or more of the following definitions may apply. In other forms of the present technology, alternative definitions may apply.

### 5.8.1 General

Air: In certain forms of the present technology, air may be taken to mean atmospheric air, and in other forms of the present technology air may be taken to mean some other combination of breathable gases, e.g. oxygen enriched air.

Ambient: In certain forms of the present technology, the term ambient will be taken to mean (i) external of the treatment system or patient, and (ii) immediately surrounding the treatment system or patient.

For example, ambient humidity with respect to a humidifier may be the humidity of air immediately surrounding the humidifier, e.g. the humidity in the room where a patient is sleeping. Such ambient humidity may be different to the humidity outside the room where a patient is sleeping.

In another example, ambient pressure may be the pressure immediately surrounding or external to the body.

In certain forms, ambient (e.g., acoustic) noise may be considered to be the background noise level in the room where a patient is located, other than for example, noise generated by an RPT device or emanating from a mask or patient interface. Ambient noise may be generated by sources outside the room.

Automatic Positive Airway Pressure (APAP) therapy: CPAP therapy in which the treatment pressure is automatically adjustable, e.g. from breath to breath, between minimum and maximum limits, depending on the presence or absence of indications of SDB events.

Continuous Positive Airway Pressure (CPAP) therapy: Respiratory pressure therapy in which the treatment pressure is approximately constant through a respiratory cycle of a patient. In some forms, the pressure at the entrance to the airways will be slightly higher during exhalation, and slightly lower during inhalation. In some forms, the pressure will vary between different respiratory cycles of the patient, for example, being increased in response to detection of indications of partial upper airway obstruction, and decreased in the absence of indications of partial upper airway obstruction.

Flow rate: The volume (or mass) of air delivered per unit time. Flow rate may refer to an instantaneous quantity. In some cases, a reference to flow rate will be a reference to a scalar quantity, namely a quantity having magnitude only. In other cases, a reference to flow rate will be a reference to a vector quantity, namely a quantity having both magnitude and direction. Flow rate may be given the symbol Q. ‘Flow rate’ is sometimes shortened to simply ‘flow’ or ‘airflow’.

In the example of patient respiration, a flow rate may be nominally positive for the inspiratory portion of a breathing cycle of a patient, and hence negative for the expiratory portion of the breathing cycle of a patient. Device flow rate, Qd, is the flow rate of air leaving the RPT device. Total flow rate, Qt, is the flow rate of air and any supplementary gas reaching the patient interface via the air circuit. Vent flow rate, Qv, is the flow rate of air leaving a vent to allow washout of exhaled gases. Leak flow rate, Ql, is the flow rate of leak from a patient interface system or elsewhere. Respiratory flow rate, Qr, is the flow rate of air that is received into the patient's respiratory system.

Flow therapy: Respiratory therapy comprising the delivery of a flow of air to an entrance to the airways at a controlled flow rate referred to as the treatment flow rate that is typically positive throughout the patient's breathing cycle.

Humidifier: The word humidifier will be taken to mean a humidifying apparatus constructed and arranged, or configured with a physical structure to be capable of providing a therapeutically beneficial amount of water (H₂O) vapour to a flow of air to ameliorate a medical respiratory condition of a patient.

Leak: The word leak will be taken to be an unintended flow of air. In one example, leak may occur as the result of an incomplete seal between a mask and a patient's face. In another example leak may occur in a swivel elbow to the ambient.

Noise, conducted (acoustic): Conducted noise in the present document refers to noise which is carried to the patient by the pneumatic path, such as the air circuit and the patient interface as well as the air therein. In one form, conducted noise may be quantified by measuring sound pressure levels at the end of an air circuit.

Noise, radiated (acoustic): Radiated noise in the present document refers to noise which is carried to the patient by the ambient air. In one form, radiated noise may be quantified by measuring sound power/pressure levels of the object in question according to ISO 3744.

Noise, vent (acoustic): Vent noise in the present document refers to noise which is generated by the flow of air through any vents such as vent holes of the patient interface.

Patient: A person, whether or not they are suffering from a respiratory condition.

Pressure: Force per unit area. Pressure may be expressed in a range of units, including cmH₂O, g-f/cm² and hectopascal. 1 cmH₂O is equal to 1 g-f/cm² and is approximately 0.98 hectopascal (1 hectopascal=100 Pa=100 N/m²=1 millibar˜0.001 atm). In this specification, unless otherwise stated, pressure is given in units of cmH₂O.

The pressure in the patient interface is given the symbol Pm, while the treatment pressure, which represents a target value to be achieved by the interface pressure Pm at the current instant of time, is given the symbol Pt.

Respiratory Pressure Therapy: The application of a supply of air to an entrance to the airways at a treatment pressure that is typically positive with respect to atmosphere.

Ventilator: A mechanical device that provides pressure support to a patient to perform some or all of the work of breathing.

### 5.8.1.1 Materials

Silicone or Silicone Elastomer: A synthetic rubber. In this specification, a reference to silicone is a reference to liquid silicone rubber (LSR) or a compression moulded silicone rubber (CMSR). One form of commercially available LSR is SILASTIC (included in the range of products sold under this trademark), manufactured by Dow Corning. Another manufacturer of LSR is Wacker. Unless otherwise specified to the contrary, an exemplary form of LSR has a Shore A (or Type A) indentation hardness in the range of about 35 to about 45 as measured using ASTM D2240.

Polycarbonate: a thermoplastic polymer of Bisphenol-A Carbonate.

### 5.8.1.2 Mechanical Properties

Resilience: Ability of a material to absorb energy when deformed elastically and to release the energy upon unloading.

Resilient: Will release substantially all of the energy when unloaded. Includes e.g. certain silicones, and thermoplastic elastomers.

Hardness: The ability of a material per se to resist deformation (e.g. described by a Young's Modulus, or an indentation hardness scale measured on a standardised sample size).
‘Soft’ materials may include silicone or thermo-plastic elastomer (TPE), and may, e.g. readily deform under finger pressure.
‘Hard’ materials may include polycarbonate, polypropylene, steel or aluminium, and may not e.g. readily deform under finger pressure.

Stiffness (or rigidity) of a structure or component: The ability of the structure or component to resist deformation in response to an applied load. The load may be a force or a moment, e.g. compression, tension, bending or torsion. The structure or component may offer different resistances in different directions. The inverse of stiffness is flexibility.

Floppy structure or component: A structure or component that will change shape, e.g. bend, when caused to support its own weight, within a relatively short period of time such as 1 second.

Rigid structure or component: A structure or component that will not substantially change shape when subject to the loads typically encountered in use. An example of such a use may be setting up and maintaining a patient interface in sealing relationship with an entrance to a patient's airways, e.g. at a load of approximately 20 to 30 cmH₂O pressure.

As an example, an I-beam may comprise a different bending stiffness (resistance to a bending load) in a first direction in comparison to a second, orthogonal direction. In another example, a structure or component may be floppy in a first direction and rigid in a second direction.

### 5.8.2 Respiratory Cycle

Apnea: According to some definitions, an apnea is said to have occurred when flow falls below a predetermined threshold for a duration, e.g. 10 seconds. An obstructive apnea will be said to have occurred when, despite patient effort, some obstruction of the airway does not allow air to flow. A central apnea will be said to have occurred when an apnea is detected that is due to a reduction in breathing effort, or the absence of breathing effort, despite the airway being patent. A mixed apnea occurs when a reduction or absence of breathing effort coincides with an obstructed airway.

Breathing rate: The rate of spontaneous respiration of a patient, usually measured in breaths per minute.

Duty cycle: The ratio of inhalation time, Ti to total breath time, Ttot.

Effort (breathing): The work done by a spontaneously breathing person attempting to breathe.

Expiratory portion of a breathing cycle: The period from the start of expiratory flow to the start of inspiratory flow.

Flow limitation: Flow limitation will be taken to be the state of affairs in a patient's respiration where an increase in effort by the patient does not give rise to a corresponding increase in flow. Where flow limitation occurs during an inspiratory portion of the breathing cycle it may be described as inspiratory flow limitation. Where flow limitation occurs during an expiratory portion of the breathing cycle it may be described as expiratory flow limitation.

Types of flow limited inspiratory waveforms:

(i) Flattened: Having a rise followed by a relatively flat portion, followed by a fall.

(ii) M-shaped: Having two local peaks, one at the leading edge, and one at the trailing edge, and a relatively flat portion between the two peaks.

(iii) Chair-shaped: Having a single local peak, the peak being at the leading edge, followed by a relatively flat portion.

(iv) Reverse-chair shaped: Having a relatively flat portion followed by single local peak, the peak being at the trailing edge.

Hypopnea: According to some definitions, a hypopnea is taken to be a reduction in flow, but not a cessation of flow. In one form, a hypopnea may be said to have occurred when there is a reduction in flow below a threshold rate for a duration. A central hypopnea will be said to have occurred when a hypopnea is detected that is due to a reduction in breathing effort. In one form in adults, either of the following may be regarded as being hypopneas:
(i) a 30% reduction in patient breathing for at least 10 seconds plus an associated 4% desaturation; or
(ii) a reduction in patient breathing (but less than 50%) for at least 10 seconds, with an associated desaturation of at least 3% or an arousal.

Hyperpnea: An increase in flow to a level higher than normal.

Inspiratory portion of a breathing cycle: The period from the start of inspiratory flow to the start of expiratory flow will be taken to be the inspiratory portion of a breathing cycle.

Patency (airway): The degree of the airway being open, or the extent to which the airway is open. A patent airway is open. Airway patency may be quantified, for example with a value of one (1) being patent, and a value of zero (0), being closed (obstructed).

Positive End-Expiratory Pressure (PEEP): The pressure above atmosphere in the lungs that exists at the end of expiration.

Peak flow rate (Qpeak): The maximum value of flow rate during the inspiratory portion of the respiratory flow waveform.

Respiratory flow rate, patient airflow rate, respiratory airflow rate (Qr): These terms may be understood to refer to the RPT device's estimate of respiratory flow rate, as opposed to “true respiratory flow rate” or “true respiratory flow rate”, which is the actual respiratory flow rate experienced by the patient, usually expressed in litres per minute.

Tidal volume (Vt): The volume of air inhaled or exhaled during normal breathing, when extra effort is not applied. In principle the inspiratory volume Vi (the volume of air inhaled) is equal to the expiratory volume Ve (the volume of air exhaled), and therefore a single tidal volume Vt may be defined as equal to either quantity. In practice the tidal volume Vt is estimated as some combination, e.g. the mean, of the inspiratory volume Vi and the expiratory volume Ve.

(inhalation) Time (Ti): The duration of the inspiratory portion of the respiratory flow rate waveform.

(exhalation) Time (Te): The duration of the expiratory portion of the respiratory flow rate waveform.

(total) Time (Ttot): The total duration between the start of one inspiratory portion of a respiratory flow rate waveform and the start of the following inspiratory portion of the respiratory flow rate waveform.

Typical recent ventilation: The value of ventilation around which recent values of ventilation Vent over some predetermined timescale tend to cluster, that is, a measure of the central tendency of the recent values of ventilation.

Upper airway obstruction (UAO): includes both partial and total upper airway obstruction. This may be associated with a state of flow limitation, in which the flow rate increases only slightly or may even decrease as the pressure difference across the upper airway increases (Starling resistor behaviour).

Ventilation (Vent): A measure of a rate of gas being exchanged by the patient's respiratory system. Measures of ventilation may include one or both of inspiratory and expiratory flow, per unit time. When expressed as a volume per minute, this quantity is often referred to as “minute ventilation”. Minute ventilation is sometimes given simply as a volume, understood to be the volume per minute.

### 5.8.3 Ventilation

Adaptive Servo-Ventilator (ASV): A servo-ventilator that has a changeable, rather than fixed target ventilation. The changeable target ventilation may be learned from some characteristic of the patient, for example, a respiratory characteristic of the patient.

Backup rate: A parameter of a ventilator that establishes the minimum breathing rate (typically in number of breaths per minute) that the ventilator will deliver to the patient, if not triggered by spontaneous respiratory effort.

Cycled: The termination of a ventilator's inspiratory phase. When a ventilator delivers a breath to a spontaneously breathing patient, at the end of the inspiratory portion of the breathing cycle, the ventilator is said to be cycled to stop delivering the breath.

Expiratory positive airway pressure (EPAP): a base pressure, to which a pressure varying within the breath is added to produce the desired interface pressure which the ventilator will attempt to achieve at a given time.

End expiratory pressure (EEP): Desired interface pressure which the ventilator will attempt to achieve at the end of the expiratory portion of the breath. If the pressure waveform template Π(Φ) is zero-valued at the end of expiration, i.e. Π(Φ)=0 when Φ=1, the EEP is equal to the EPAP.

Inspiratory positive airway pressure (IPAP): Maximum desired interface pressure which the ventilator will attempt to achieve during the inspiratory portion of the breath.

Pressure support: A number that is indicative of the increase in pressure during ventilator inspiration over that during ventilator expiration, and generally means the difference in pressure between the maximum value during inspiration and the base pressure (e.g., PS=IPAP−EPAP). In some contexts pressure support means the difference which the ventilator aims to achieve, rather than what it actually achieves.

Servo-ventilator: A ventilator that measures patient ventilation, has a target ventilation, and which adjusts the level of pressure support to bring the patient ventilation towards the target ventilation.

Spontaneous/Timed (S/T): A mode of a ventilator or other device that attempts to detect the initiation of a breath of a spontaneously breathing patient. If however, the device is unable to detect a breath within a predetermined period of time, the device will automatically initiate delivery of the breath.

Swing: Equivalent term to pressure support.

Triggered: When a ventilator, or other respiratory therapy device such as an RPT device or portable oxygen concentrator, delivers a volume of breathable gas to a spontaneously breathing patient, it is said to be triggered to do so. Triggering usually takes place at or near the initiation of the respiratory portion of the breathing cycle by the patient's efforts.

### 5.8.4 Interface Structures

Elbow: An elbow is an example of a structure that directs an axis of flow of air travelling therethrough to change direction through an angle. In one form, the angle may be approximately 90 degrees. In another form, the angle may be more, or less than 90 degrees. The elbow may have an approximately circular cross-section. In another form the elbow may have an oval or a rectangular cross-section. In certain forms an elbow may be rotatable with respect to a mating component, e.g. about 360 degrees. In certain forms an elbow may be removable from a mating component, e.g. via a snap connection. In certain forms, an elbow may be assembled to a mating component via a one-time snap during manufacture, but not removable by a patient.

Membrane: Membrane will be taken to mean a typically thin element that has, preferably, substantially no resistance to bending, but has resistance to being stretched.

Seal: May be a noun form (“a seal”) which refers to a structure, or a verb form (“to seal”) which refers to the effect. Two elements may be constructed and/or arranged to ‘seal’ or to effect ‘sealing’ therebetween without requiring a separate ‘seal’ element per se.

Swivel (noun): A subassembly of components configured to rotate about a common axis, preferably independently, preferably under low torque. In one form, the swivel may be constructed to rotate through an angle of at least 360 degrees. In another form, the swivel may be constructed to rotate through an angle less than 360 degrees. When used in the context of an air delivery conduit, the sub-assembly of components preferably comprises a matched pair of cylindrical conduits. There may be little or no leak flow of air from the swivel in use.

### 5.8.5 Shape of Structures

Products in accordance with the present technology may comprise one or more three-dimensional mechanical structures, for example a mask cushion or an impeller. The three-dimensional structures may be bounded by two-dimensional surfaces. These surfaces may be distinguished using a label to describe an associated surface orientation, location, function, or some other characteristic. For example a structure may comprise one or more of an anterior surface, a posterior surface, an interior surface and an exterior surface. In another example, a seal-forming structure may comprise a face-contacting (e.g. outer) surface, and a separate non-face-contacting (e.g. underside or inner) surface. In another example, a structure may comprise a first surface and a second surface.

To facilitate describing the shape of the three-dimensional structures and the surfaces, we first consider a cross-section through a surface of the structure at a point, p. See FIG. 3B to FIG. 3F, which illustrate examples of cross-sections at point p on a surface, and the resulting plane curves. FIGS. 3B to 3F also illustrate an outward normal vector at p. The outward normal vector at p points away from the surface. In some examples we describe the surface from the point of view of an imaginary small person standing upright on the surface.

### 5.8.5.1 Curvature in One Dimension

The curvature of a plane curve at p may be described as having a sign (e.g. positive, negative) and a magnitude (e.g. 1/radius of a circle that just touches the curve at p).

Positive curvature: If the curve at p turns towards the outward normal, the curvature at that point will be taken to be positive (if the imaginary small person leaves the point p they must walk uphill). See FIG. 3B (relatively large positive curvature compared to FIG. 3C) and FIG. 3C (relatively small positive curvature compared to FIG. 3B). Such curves are often referred to as concave.

Zero curvature: If the curve at p is a straight line, the curvature will be taken to be zero (if the imaginary small person leaves the point p, they can walk on a level, neither up nor down). See FIG. 3D.

Negative curvature: If the curve at p turns away from the outward normal, the curvature in that direction at that point will be taken to be negative (if the imaginary small person leaves the point p they must walk downhill) See FIG. 3E (relatively small negative curvature compared to FIG. 3F) and FIG. 3F (relatively large negative curvature compared to FIG. 3E). Such curves are often referred to as convex.

### 5.8.5.2 Curvature of Two Dimensional Surfaces

A description of the shape at a given point on a two-dimensional surface in accordance with the present technology may include multiple normal cross-sections. The multiple cross-sections may cut the surface in a plane that includes the outward normal (a “normal plane”), and each cross-section may be taken in a different direction. Each cross-section results in a plane curve with a corresponding curvature. The different curvatures at that point may have the same sign, or a different sign. Each of the curvatures at that point has a magnitude, e.g. relatively small. The plane curves in FIGS. 3B to 3F could be examples of such multiple cross-sections at a particular point.

Principal curvatures and directions: The directions of the normal planes where the curvature of the curve takes its maximum and minimum values are called the principal directions. In the examples of FIG. 3B to FIG. 3F, the maximum curvature occurs in FIG. 3B, and the minimum occurs in FIG. 3F, hence FIG. 3B and FIG. 3F are cross sections in the principal directions. The principal curvatures at p are the curvatures in the principal directions.

Region of a surface: A connected set of points on a surface. The set of points in a region may have similar characteristics, e.g. curvatures or signs.

Saddle region: A region where at each point, the principal curvatures have opposite signs, that is, one is positive, and the other is negative (depending on the direction to which the imaginary person turns, they may walk uphill or downhill)

Dome region: A region where at each point the principal curvatures have the same sign, e.g. both positive (a “concave dome”) or both negative (a “convex dome”).

Cylindrical region: A region where one principal curvature is zero (or, for example, zero within manufacturing tolerances) and the other principal curvature is non-zero.

Planar region: A region of a surface where both of the principal curvatures are zero (or, for example, zero within manufacturing tolerances).

Edge of a surface: A boundary or limit of a surface or region.

Path: In certain forms of the present technology, ‘path’ will be taken to mean a path in the mathematical—topological sense, e.g. a continuous space curve from f(0) to f(1) on a surface. In certain forms of the present technology, a ‘path’ may be described as a route or course, including e.g. a set of points on a surface. (The path for the imaginary person is where they walk on the surface, and is analogous to a garden path).

Path length: In certain forms of the present technology, ‘path length’ will be taken to mean the distance along the surface from f(0) to f(1), that is, the distance along the path on the surface. There may be more than one path between two points on a surface and such paths may have different path lengths. (The path length for the imaginary person would be the distance they have to walk on the surface along the path).

Straight-line distance: The straight-line distance is the distance between two points on a surface, but without regard to the surface. On planar regions, there would be a path on the surface having the same path length as the straight-line distance between two points on the surface. On non-planar surfaces, there may be no paths having the same path length as the straight-line distance between two points. (For the imaginary person, the straight-line distance would correspond to the distance ‘as the crow flies’.)

### 5.8.5.3 Space Curves

Space curves: Unlike a plane curve, a space curve does not necessarily lie in any particular plane. A space curve may be closed, that is, having no endpoints. A space curve may be considered to be a one-dimensional piece of three-dimensional space. An imaginary person walking on a strand of the DNA helix walks along a space curve. A typical human left ear comprises a helix, which is a left-hand helix, see FIG. 3L. A typical human right ear comprises a helix, which is a right-hand helix, see FIG. 3M. FIG. 3N shows a right-hand helix. The edge of a structure, e.g. the edge of a membrane or impeller, may follow a space curve. In general, a space curve may be described by a curvature and a torsion at each point on the space curve. Torsion is a measure of how the curve turns out of a plane. Torsion has a sign and a magnitude. The torsion at a point on a space curve may be characterised with reference to the tangent, normal and binormal vectors at that point.

Tangent unit vector (or unit tangent vector): For each point on a curve, a vector at the point specifies a direction from that point, as well as a magnitude. A tangent unit vector is a unit vector pointing in the same direction as the curve at that point. If an imaginary person were flying along the curve and fell off her vehicle at a particular point, the direction of the tangent vector is the direction she would be travelling.

Unit normal vector: As the imaginary person moves along the curve, this tangent vector itself changes. The unit vector pointing in the same direction that the tangent vector is changing is called the unit principal normal vector. It is perpendicular to the tangent vector.

Binormal unit vector: The binormal unit vector is perpendicular to both the tangent vector and the principal normal vector. Its direction may be determined by a right-hand rule (see e.g. FIG. 3K), or alternatively by a left-hand rule (FIG. 3J).

Osculating plane: The plane containing the unit tangent vector and the unit principal normal vector. See FIGS. 3O and 3P.

Torsion of a space curve: The torsion at a point of a space curve is the magnitude of the rate of change of the binormal unit vector at that point. It measures how much the curve deviates from the osculating plane. A space curve which lies in a plane has zero torsion. A space curve which deviates a relatively small amount from the osculating plane will have a relatively small magnitude of torsion (e.g. a gently sloping helical path). A space curve which deviates a relatively large amount from the osculating plane will have a relatively large magnitude of torsion (e.g. a steeply sloping helical path). With reference to FIG. 3N, since T2>T1, the magnitude of the torsion near the top coils of the helix of FIG. 3N is greater than the magnitude of the torsion of the bottom coils of the helix of FIG. 3N.

With reference to the right-hand rule of FIG. 3K, a space curve turning towards the direction of the right-hand binormal may be considered as having a right-hand positive torsion (e.g. a right-hand helix as shown in FIG. 3N). A space curve turning away from the direction of the right-hand binormal may be considered as having a right-hand negative torsion (e.g. a left-hand helix).

Equivalently, and with reference to a left-hand rule (see FIG. 3J), a space curve turning towards the direction of the left-hand binormal may be considered as having a left-hand positive torsion (e.g. a left-hand helix). Hence left-hand positive is equivalent to right-hand negative.

### 5.8.5.4 Holes

A surface may have a one-dimensional hole, e.g. a hole bounded by a plane curve or by a space curve. Thin structures (e.g. a membrane) with a hole, may be described as having a one-dimensional hole. See for example the one dimensional hole in the surface of structure shown in FIG. 3G, bounded by a plane curve.

A structure may have a two-dimensional hole, e.g. a hole bounded by a surface. For example, an inflatable tyre has a two dimensional hole bounded by the interior surface of the tyre. In another example, a bladder with a cavity for air or gel could have a two-dimensional hole.

### 5.9 Other Remarks

A portion of the disclosure of this patent document contains material which is subject to copyright protection. The copyright owner has no objection to the facsimile reproduction by anyone of the patent document or the patent disclosure, as it appears in Patent Office patent files or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated herein by reference in their entirety to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular examples, it is to be understood that these examples are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative examples and that other arrangements may be devised without departing from the spirit and scope of the technology.

### 5.10 Reference Signs List

| patient | 1000 |
|---|---|
| bed partner | 1100 |
| patient interface | 3000 |
| seal-forming structure | 3100 |
| plenum chamber | 3200 |
| structure | 3300 |
| vent | 3400 |
| connection port | 3600 |
| forehead support | 3700 |
| RPT device | 4000 |
| external housing | 4010 |
| upper portion | 4012 |
| portion | 4014 |
| panel | 4015 |
| chassis | 4016 |
| handle | 4018 |
| pneumatic block | 4020 |
| air filter | 4110 |
| inlet air filter | 4112 |
| outlet air filter | 4114 |
| muffler | 4120 |
| inlet muffler | 4122 |
| outlet muffler | 4124 |
| pressure generator | 4140 |
| blower | 4142 |
| motor | 4144 |
| anti-spill back valve | 4160 |
| air circuit | 4170 |
| air circuit | 4171 |
| electrical components | 4200 |
| Printed Circuit Board Assembly | 4202 |
| power supply | 4210 |
| input device | 4220 |
| transducer | 4270 |
| humidifier | 5000 |
| humidifier inlet | 5002 |
| humidifier outlet | 5004 |
| humidifier base | 5006 |
| reservoir | 5110 |
| conductive portion | 5120 |
| humidifier chamber | 5130 |
| locking lever | 5135 |
| water level indicator | 5150 |
| humidifier transducer | 5210 |
| air pressure sensor | 5212 |
| air flow rate transducer | 5214 |
| temperature sensor | 5216 |
| humidity sensor | 5218 |
| heating element | 5240 |
| humidifier controller | 5250 |
| central humidifier controller | 5251 |
| heating element controller | 5252 |
| air circuit controller | 5254 |
| humidifier | 7000 |
| respiratory apparatus | 7001 |
| monitoring apparatus | 7100 |
| water reservoir | 7110 |
| conductive portion | 7120 |
| chamber | 7130 |
| tub base | 7140 |
| tub lid | 7144 |
| complimentary features | 7145 |
| complimentary features | 7146 |
| tub bottom surface | 7148 |
| side wall | 7152 |
| opening | 7156 |
| outlet | 7160 |
| tub emptying aperture | 7164 |
| channel | 7168 |
| outlet | 7172 |
| chamber base | 7176 |
| chamber bottom surface | 7180 |
| insulation layer | 7184 |
| chamber side wall | 7188 |
| chamber opening | 7192 |
| sealing member | 7196 |
| connectors | 7200 |
| electrical connector | 7202 |
| lid closure member | 7204 |
| latches | 7206 |
| lid opening member | 7208 |
| entry chamber | 7238 |
| heating element | 7240 |
| hinge portions | 7260 |
| humidifier chamber outlet | 7264 |
| chamber lid | 7268 |
| hinge portion | 7272 |
| catches | 7276 |
| window | 7280 |
| lid seal | 7284 |
| sealing rim | 7285 |
| wedge-shaped protrusions | 7288 |
| domed portion | 7292 |
| sealing ring | 7296 |
| inner sealing rim | 7300 |
| aperture | 7304 |
| airflow pathway | 7308 |
| upper seal | 7312 |
| lower seal | 7316 |
| wall | 7320 |
| entry seal | 7322 |
| entry opening | 7324 |
| entry chamber | 7328 |
| pathway seal | 7332 |
| deflector wall | 7336 |
| passage entryway | 7340 |
| airflow | 7400 |
| humidifier | 17000 |
| water reservoir | 17110 |
| chamber | 17130 |
| tub base | 17140 |
| tub lid | 17144 |
| surface | 17148 |
| tub inlet | 17160 |
| tub outlet | 17172 |
| chamber base | 17176 |
| chamber side wall | 17188 |
| chamber inlet | 17192 |
| seal | 17196 |
| heating element | 17240 |
| chamber outlet | 17264 |
| airflow | 17400 |
| groove | 17404 |
| lateral side | 17408 |
| deflector seal | 17412 |
| sealing surface | 17416 |
| dividing gasket | 17420 |
| pathway seal | 17424 |
| end portion | 17426 |
| skirt | 17428 |

1. An apparatus for providing a humidified flow of pressurized breathable gas to be delivered to a patient, the apparatus comprising:
a humidification chamber having a heatable base surface and at least one chamber side wall extending from the heatable base surface;
a humidification tub configured to contain a supply of water, the humidification tub including a thermally conductive base and at least one tub side wall extending from the thermally conductive base, the humidification tub being removably positionable at least partially within the humidification chamber where the thermally conductive base contacts the heatable base surface, so as to allow heating of the water in the humidification tub, the humidification tub being configured to receive the humidified flow of pressurized breathable gas and output the flow of pressurized breathable gas with increased humidity;
wherein the arrangement is such that the flow of pressurized breathable gas is configured to, after entering the humidification chamber and before entering the humidification tub, follow a pathway along at least a portion of a perimeter of the humidification tub, the pathway being located inside the humidification chamber but outside the humidification tub, the propagation of the pressurized breathable gas along the pathway causing the pressurized breathable gas to be pre-heated in the humidification chamber before entering the humidification tub.

2. The apparatus of claim 1, wherein the pathway is formed to be in a lower portion of the humidification chamber and in the proximity of the heatable base surface.

3. The apparatus of claim 1, wherein the airflow is directed to propagate along the pathway.

4. The apparatus of claim 1, wherein the pathway is formed between the at least one heatable base surface or chamber side wall and the thermally conductive base or tub side wall, the flow of pressurized breathable gas is configured to flow along at least a portion of a perimeter of the humidification tub through the pathway.

5. The apparatus of claim 4, wherein the flow of pressurized breathable gas is directed to flow through the pathway along at least a portion of the perimeter of at least a lower portion of the humidification tub.

6. The apparatus of claim 5, wherein an entrance of the pathway is disposed at least 180° around the perimeter of the humidification tub from an exit of the pathway.

7. The apparatus of claim 6, wherein the entrance of the pathway is disposed approximately 360° around the perimeter of the humidification tub from the exit of the pathway.

8. The apparatus of claim 2, wherein the pathway is at least partially defined by a first pathway seal, the first pathway seal contacting at least portions of the at least one chamber side wall and the at least one tub side wall in a sealing arrangement in use.

9. The apparatus of claim 8, wherein the first pathway seal is a face seal.

10. The apparatus of claim 8, wherein the first pathway seal is integrally formed on the chamber side wall and is configured to contact the tub side wall when the humidification tub is positioned within the humidification chamber.

11. The apparatus of claim 10, wherein each of the chamber side wall and the tub side wall is a side wall or a face wall.

12. The apparatus of claim 11, wherein at least a portion of the at least one chamber side wall is perpendicular to the heatable base surface and at least a portion of the at least one tub side wall is perpendicular to the thermally conductive base, and wherein the portion of the at least one chamber side wall and the portion of the at least one tub side wall are configured to be vertically oriented in use.

13. The apparatus of claim 8, wherein the first pathway seal forms a superior boundary of the pathway in use.

14. The apparatus of claim 8, wherein the first pathway seal is more inferior than a chamber opening, in use, the chamber opening being configured to receive the flow of pressurized breathable gas from a flow generator.

15. The apparatus of claim 8, wherein the first pathway seal is configured to direct the flow of pressurized breathable gas along the pathway toward an inlet of the humidification tub.

16. The apparatus of claim 8, further comprising a second pathway seal spaced apart from the first pathway seal and contacting the at least one chamber side wall and the at least one tub side wall in a sealing arrangement, in use.

17. The apparatus of claim 16, wherein the second pathway seal forms an inferior boundary of the pathway in use.

18. The apparatus of claim 16, wherein the second pathway seal is a face seal.

19. The apparatus of claim 16, wherein the second pathway seal is integrally formed on the at least one chamber side wall, and is configured to contact a chamfered edge of the at least one tub side wall when the humidification tub is positioned within the humidification chamber.

20. The apparatus of claim 16, wherein the second pathway seal contacts the heatable base surface, in use.

21. The apparatus of claim 4, further comprising a bypass seal disposed proximate to a pathway entrance, and configured to form a flow path in a single direction along the pathway.

22. The apparatus of claim 4, wherein a first width between the at least one chamber side wall and the at least one tub side wall at a pathway entrance is less than a second width between the at least one chamber side wall and the at least one tub side wall at a pathway exit.

23. The apparatus of claim 4, wherein the humidification chamber further includes a chamber lid hingedly attached to the at least one chamber side wall and pivotably movable between an open position and a closed position.

24. The apparatus of claim 23, wherein the chamber lid includes an entry opening configured to cooperate with a pathway exit when the chamber lid is in the closed position.

25. The apparatus of claim 24, wherein the entry opening includes a substantially vertical portion and a substantially horizontal portion, the substantially vertical portion configured to extend into the pathway exit, and the substantially horizontal portion extending at least partially over a tub opening configured to provide communication to an interior of the humidification tub.

26. The apparatus of claim 24, wherein in an operational configuration the entry opening is located superior to the pathway, in use.

27. The apparatus of claim 23, further comprising a lid closure assembly to selectively lock the chamber lid to the at least one chamber side wall.

28. The apparatus of claim 27, wherein the lid closure assembly includes a lid opening member, a spring, and a latch.

29. The apparatus of claim 28, wherein the lid opening member is slidably coupled to the at least one chamber side wall to move the latch between a locked position and an unlocked position, the latch being configured to mechanically engage a catch of the chamber lid in the locked position in order to retain the chamber lid in the closed position, the spring biasing the latch into the locked position.

30. The apparatus of claim 1, further comprising a chamber wall seal surrounding the chamber opening.

31. The apparatus of claim 1, wherein the thermally conductive base includes a metal surface.

32. The apparatus of claim 1, further comprising:
a flow generator having a flow generator outlet, and being configured to supply the flow of pressurized breathable gas;
wherein:
the flow generator outlet is disposed upstream from the humidification chamber, and the flow of pressurized breathable gas is configured to flow from the flow generator outlet to the humidification chamber;
a temperature of the flow of pressurized breathable gas entering the humidification tub is higher than the temperature of the flow of pressurized breathable gas exiting the flow generator outlet.

33. The apparatus of claim 32, wherein the flow generator is integrally coupled to the humidification chamber.

34. The apparatus of claim 32, wherein the flow generator outlet contacts the chamber wall seal in a sealing arrangement.

35. The apparatus of claim 32, when dependent from claim 6, wherein in an operational configuration the flow generator outlet is located superior to the first pathway seal, in use.
